(19) United States
(12) Patent Application Publication
(10) Pub. No.: US 2025/0360199 A1
(43) Pub. Date: Nov. 27, 2025

(54) IMMUNOGENS AND METHODS FOR INDUCING AN IMMUNE RESPONSE

(71) Applicant: The United States of America, as represented by the Secretary, Dept. of Health and Human Services, Bethesda, MD (US)

(72) Inventors: Barbara K. Felber, Rockville, MD (US); George N. Pavlakis, Rockville, MD (US)

(21) Appl. No.: 18/874,188

(22) PCT Filed: Jun. 26, 2023
(86) PCT No.: PCT/US2023/069057

§ 371 Date: Dec. 12, 2024

Related U.S. Application Data
(60) Provisional application No. 63/358,919, filed on Jul. 7, 2022.

(51) Int. Cl. A61K 39/21; A61K 38/20; A61K 39/00; A61P 37/04; C07K 14/005

(52) U.S. Cl. CPC A61K 39/21; A61K 38/20; A61K 38/2046; A61K 38/208; A61K 38/2086; A61P 37/04; C07K 14/005; A61K 2039/53; A61K 2039/6018; C12N 2740/16034; C12N 2740/16222; C12N 2740/16271

(57) ABSTRACT

This disclosure generally relates to methods and compositions for eliciting broad and robust immune responses to a protein of interest. The methods employ both DNA and RNA-based vaccines that encode at least a portion of the protein of interest.

### CROSS-REFERENCE TO RELATED APPLICATIONS

This application claims the benefit of U.S. Provisional Application No. 63/358,919, filed Jul. 7, 2022, which is incorporated by reference herein in its entirety.

### STATEMENT REGARDING FEDERALLY SPONSORED RESEARCH

This work was made with government support under National Institutes of Health, National Institutes of Health award AI027757. The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Jun. 22, 2023, is named “22-1034-WO_Sequence-Listing.xml” and is 17,321 bytes in size.

### BACKGROUND

### Field of the Disclosure

This disclosure generally relates to methods and compositions for eliciting broad and robust immune responses to a protein of interest. The methods employ nucleic acid (both DNA and RNA) vaccines that encode the protein of interest.

### Description of Related Art

The introduction of highly efficient antiretroviral drugs (ART) for the treatment of HIV infection dramatically improved the disease prognosis and extended the life expectancy of infected individuals [reviewed in (1-6)]. Nevertheless, ART fails to eradicate infected cells, and upon ART discontinuation, viral rebound occurs within 2-4 weeks. Therefore, life-long continuous ART is required to prevent disease progression. To eliminate the burden of chronic drug consumption and associated long-term toxicities, immune therapeutic strategies aiming to eliminate the long-term reservoir of HIV-infected cells or achieve a functional cure are being explored.

Therapeutic vaccination has a potential role either as a component of a strategy to eliminate cells latently infected with HIV-1 (reduction of latent reservoir), or as a functional cure to achieve permanent host control of HIV-1 infection to undetectable levels off ART without complete eradication of the latent reservoir (7-9). Due to control of virus replication under ART, only very low or no virus-specific T cell responses are present in the circulation. An effective therapeutic HIV-1 vaccine should induce potent cytotoxic T cell responses which could contribute to control of viremia and thereby reduce the pool of infected cells. CD8+ T cell immune responses induced upon therapeutic vaccination during ART can contribute to control viral replication upon treatment interruption [reviewed in (6-8, 10-13)].

The concept of directing T cell responses towards conserved regions in the HIV proteome has been studied, and an approach uses a DNA-based vaccine platform to target conserved elements (CE) within HIV-1 p24Gag (see references 13-26; the disclosures of which are each incorporated by reference herein in their entirety). CE were selected following stringent criteria: (i) more than 98% conservation among the known HIV-1 sequences, (ii) prevalent recognition by long-term non-progressor HIV-infected individuals, and (iii) encoding of conserved epitopes with very broad HLA coverage at the population level. These studies showed that mutations in Gag CE are much more likely to disable virus replication in cell culture than mutations outside of CE (26-28). The studies also demonstrated that vaccination with plasmid DNA encoding these CE epitopes is immunogenic in murine and NHP models (15, 16, 29). It was reported that CE vaccination regimens that modified the hierarchy of T cell epitope recognition otherwise imposed by the dominant variable regions within the full-length viral proteins (16, 30). These optimized DNA vaccine regimens, aiming to induce an adaptive response that makes virus escape difficult, broadened epitope recognition and improved the functionality of the vaccine-induced T cell responses, eliciting cytotoxic T cells targeting conserved epitopes in immunized rhesus macaques.

Using the SIV/macaque model, it was also shown that DNA vaccines expressing homologous epitopes present in SIV p27Gag were very immunogenic (31). The T cells targeting these conserved epitopes were activated upon SIV-infection which demonstrated that the CE-specific T cells recognize infected cells in vivo. Thus, the use of immunogens encoding CE epitopes may be a promising therapeutic strategy for the management of HIV-1 infected individuals. The concept of CE vaccination has been translated into several clinical trials, including one prophylactic trial in HIV-naïve human volunteers (HVTN 119; NCT03181789) and two therapeutic trials in HIV-positive individuals on ART (ACTG A5369 [NCT03560258] and NCT04357821).

Nucleic acid-based vaccines have several significant advantages over other vaccine platforms, including streamlined and predictable scale-up production, and flexibility to enable rapid vaccine design. These features are critical in global outbreak situations and against emerging infectious diseases (locally or globally) (32). In addition, nucleic acid-based vaccines are not limited in the number of vaccinations because, in contrast with other modalities, especially viral vector-based vaccines, they do not induce immune responses targeting any vaccine component other than the intended immunogen [reviewed in (33-38)]. A putative hurdle with DNA vaccines is the delivery, which is performed by intramuscular/intradermal injection, and requires nuclear entry for immunogen expression, a process that is augmented by i.e., in vivo electroporation. In contrast, mRNA-based vaccines only require entry into the cytoplasm for translation, and this is achieved by simple needle/syringe injection. However, mRNA needs to be formulated within nanoparticles to avoid degradation and facilitate cellular uptake. LNP formulated mRNA vaccines may have an adjuvant effect by stimulating several innate immune responses and induce cytokine release shortly after immunization, which could influence the development of an efficient adaptive immune response (39). Among the nucleic acid-based vaccines, the DNA platform elicits long-lasting adaptive responses with both CD4+ and cytotoxic CD8+ T cell responses in macaques and humans (30, 40-47). mRNA vaccines are efficient in inducing humoral immunity and mainly CD4+T helper responses against several antigens (32, 35, 36, 48-51). The successful development and practical application of the mRNA technology have been showcased with the recent approval and distribution of several COVID-19 mRNA vaccines, demonstrating induction of potent anti-Spike Ab and low levels of CD4+T helper and CD8+ T cell responses in humans (50, 52-59).

No comparative studies of the two nucleic acid vaccine modalities using the same immunogens have been reported so far. As shown herein, the HIV-1 CE vaccine concept using an mRNA/LNP vaccine platform (see WO2018078053) was tested for its immunological potential as a T cell vaccine in Indian rhesus macaques. This technology comprised of chemically non-modified nucleoside synthetic mRNAs has been tested in pre-clinical and clinical trials (35, 60-63). The immunological outcome of this study was also compared to similar DNA based vaccine regimens. In addition, combinations of DNA and RNA vaccine technologies were evaluated in different prime-boost immunization studies, identifying approaches to further increase cellular immunity with promising immunological advantages.

### SUMMARY

It is against the above background that the present disclosure provides certain advantages over the prior art.

Although this disclosure as provided herein is not limited to specific advantages or functionalities, the disclosure provides combination DNA and RNA vaccine technologies in different prime-boost immunization strategies, which provide further increases in cellular immunity and immunological advantages.

In one aspect, disclosure provides a method of inducing an immune response to a protein of interest in a subject, the method comprising:
(a) administering one or more priming doses comprising a DNA construct encoding at least a portion of the protein of interest;
(b) administering one or more boosting doses comprising a lipid nanoparticle (LNP) comprising an RNA construct encoding at least a portion of the protein of interest;
wherein the one or more boosting doses is administered about 2 weeks to about 4 years after the last of the one or more priming doses.

In another aspect, this disclosure provides a method of inducing an immune response to a protein of interest in a subject, the method comprising:
(a) administering one or more priming doses comprising a lipid nanoparticle (LNP) comprising an RNA construct encoding at least a portion of the protein of interest;
(b) administering one or more boosting doses comprising a DNA construct encoding at least a portion of the protein of interest;
wherein the one or more boosting doses is administered about 2 weeks to about 4 years after the last of the one or more priming doses.

In some aspects of the methods disclosed herein, the one or more priming doses, the one or more boosting doses, or both the one or more priming doses and the one or more boosting doses comprises one or more adjuvants.

In some aspects of the methods disclosed herein, the adjuvants are selected from the group consisting of Adju-Phos™, Adjumer™, albumin-heparin microparticles, Algammulin, AS-2 adjuvant, Avridine™, B7-2, BAK, BAY R1005, Bupivacaine, Bupivacaine-HCl, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CRL1005, D-Murapalmitine, Diphtheria toxoid, DMPC, DMPG, Freund's Complete Adjuvant, GM-CSF, GMDP, hGM-CSF, hIL-12 (N222L), hTNF-alpha, IFA, Imiquimod™, ImmTher™, Interferon-gamma. Interleukin-1 beta, Interleukin-12, Interleukin-2, Interleukin-4, Interleukin-7, ISCOM(s)™, Iscoprep 7.0.3™, Loxoribine, LT (R192G), LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, S-28463, SAF-1, Sclavo peptide, Span 85, Specol, Tetanus toxoid (TT), Theramide™, Threonyl muramyl dipeptide (TMDP), and Ty Particles.

In some aspects of the methods disclosed herein, each dose of the one or more priming doses comprising the DNA construct comprises about 1 mg to about 20 mg of the DNA construct, for example about 1 mg, about 2 mg, about 2.5 mg, about 4 mg, about 5 mg, about 6 mg, about 7 mg, about 8 mg, about 9 mg, about 10 mg, about 15 mg, or about 20 mg.

In some aspects of the methods disclosed herein, each dose of the one or more boosting doses comprising the DNA construct comprises about 1 mg to about 20 mg of the DNA construct, for example about 1 mg, about 2 mg, about 2.5 mg, about 4 mg, about 5 mg, about 6 mg, about 7 mg, about 8 mg, about 9 mg, about 10 mg, about 15 mg, or about 20 mg.

In some aspects of the methods disclosed herein, each dose of the one or more boosting doses comprising the RNA construct comprises about 1 μg to about 100 μg of the RNA construct, for example about 1 μg, about 2.5 μg, about 4 μg, about 5 μg, about 6 μg, about 7 μg, about 8 μg, about 9 μg, about 10 μg, about 15 μg, about 20 μg, about 25 μg, about 30 μg, about 40 μg, about 50 μg, about 60 μg, about 70 μg, about 80 μg, about 90 μg, or about 100 μg.

In some aspects of the methods disclosed herein, each dose of the one or more priming doses comprising the RNA construct comprises about 1 μg to about 100 μg of the RNA construct, for example about 1 μg, about 2.5 μg, about 4 μg, about 5 μg, about 6 μg, about 7 μg, about 8 μg, about 9 μg, about 10 μg, about 15 μg, about 20 μg, about 25 μg, about 30 μg, about 40 μg, about 50 μg, about 60 μg, about 70 μg, about 80 μg, about 90 μg, or about 100 μg.

In some aspects of the methods disclosed herein, the one or more priming doses, the one or more boosting doses, or both the one or more priming doses and the one or more boosting doses is administered by intramuscular injection, intramuscular injection followed by in vivo electroporation, subcutaneous injection, intravenous injection, or by inhalation or intranasal.

In some aspects of the methods disclosed herein, the protein of interest is HIV-1 Gag or one or more conserved elements from HIV-1 p24^{gag} (for example as disclosed in WO2013131099 or WO2016183420, which hereby expressly incorporated by reference in their entirety).

In some aspects of the methods disclosed herein, the protein of interest encoded by the DNA construct or the RNA construct is the same protein.

In some aspects of the methods disclosed herein, the protein of interest encoded by the DNA construct or the RNA construct are different proteins, for example, comprising one or more conserved elements, fragments, or variants of the protein of interest.

In some aspects of the methods disclosed herein, the one or more priming doses comprises two, three, four, or five doses or more, each separated by at least about 2 weeks, 3 weeks, 4 weeks, 5 weeks, 6 weeks, 7 weeks, 8 weeks, or more.

In some aspects of the methods disclosed herein, the one or more boosting doses comprises two, three, four, or five doses or more, each separated by at least about 2 weeks, 3 weeks, 4 weeks, 5 weeks, 6 weeks, 7 weeks, 8 weeks, or more.

In some aspects of the methods disclosed herein, the one or more boosting doses is administered at least about 2 weeks, 3 weeks, 4 weeks, 5 weeks, 6 weeks, 7 weeks, 8 weeks, or more after the last of the one or more priming doses, or wherein the one or more boosting doses is administered at least about 3 months, 4 months, 5 months, 6 months, 7 months, 8 months, 9 months, 10 months, 11 months, 12 months or more after the last of the one or more priming doses.

This disclosure also provides a lipid nanoparticle (LNP), comprising an RNA molecule encoding HIV-1 Gag or one or more conserved elements from HIV-1 p24^{gag}.

In some aspects of the lipids disclosed herein, the LNP comprises about 1 μg to about 100 μg of the RNA molecule.

In some aspects of the lipids disclosed herein the LNP comprises a second RNA molecule encoding one or more cytokines selected from IL-12, IL-7, IL-15, and IL-21.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present disclosure can be best understood when read in conjunction with the following drawings, where like structure is indicated with like reference numerals and in which:

FIG. 1A-1H show mRNA/LNP vaccination of naïve rhesus macaques induces robust antibody and low T cell responses. (FIG. 1A) Schematic representation of the HIV-1 mRNA/LNP vaccination regimens of the three groups receiving four vaccinations (V1 to V4) with the indicated gag immunogens. (FIG. 1B) Plots showing the vaccine-induced Gag Ab measured over time as reciprocal endpoint titers (log). (FIG. 1C) Durability of CE and CE+gag mRNA/LNP vaccine induced Gag antibodies. Gag Ab titers were plotted from eight vaccinated macaques [group 1, n=3 (CE mRNA/LNP), blue symbols; group 3, n=5, red symbols; (CE/CE+Gag mRNA)] described in panel B over 62 weeks post the last vaccination (V4). Black symbols denote median antibody titers. (FIG. 1D, FIG. 1E) Antigen-specific T cell responses by flow cytometry measured two weeks after the 4th vaccination. (FIG. 1D) Gag-specific and (FIG. 1E) CE-specific memory (CD3⁺CD95+IFN-γ⁺) T cell responses were measured 2 weeks after the 4th vaccination. (FIG. 1F) Comparison of memory T cell responses in macaques immunized with the mRNA/LNP regimen (group 3) and the homologous DNA vaccine regimen from historical samples (30) at two weeks post vaccination 4. The data were obtained in the same flow cytometer (BD Fortessa) using the same antibody panel and the same gating strategy in these two groups of samples and included an internal positive control. This approach excluded any variability associated with instrument and/or reagent performance. P values are from unpaired t test (Mann-Whitney). (FIG. 1G) The CE/CE+gag DNA vaccine (dose: 2 mg prime, 2+2 mg boost) contained IL-12 DNA as vaccine adjuvant and was administered by IM injection followed by electroporation using the same schedule for the matching the mRNA/LNP. Plot showing Gag Ab responses after the 4th vaccination. The last time points of blood collection were weeks 70 and 76, respectively, for 3 animals each and these time points were combined plotted as week 73. (FIG. 1H) Comparison of Gag antibody titers (log) in macaques receiving CE/CE+Gag vaccine as mRNA/LNP (wk 62) and DNA (wk 73) vaccine post the 4th vaccination, respectively.

FIG. 2A-2G show that high dose mRNA/LNP vaccination increased cellular but not humoral responses. (FIG. 2A) Schematic representation of the high-dose (100 μg) gag mRNA/LNP vaccination regimen administered in two vaccinations (V1, V2). The data were compared to the low dose (25 μg) regimen described in FIG. 1 (group 2). (FIG. 2B) Vaccine-induced Gag Ab titers were plotted over time as reciprocal endpoint titers (log) from macaques immunized with the high dose mRNA/LNP vaccine (100 μg). (FIG. 2C) Comparison of Gag Ab titers from the high and low dose mRNA/LNP regimens at two weeks after the 2nd vaccination. (FIG. 2D, FIG. 2E) The antigen-specific cellular analysis was performed by flow cytometry at 2 weeks after the 2nd vaccination. (FIG. 2D) Plot showing the Gag-specific CD4+ and CD8+ memory (CD3⁺CD95*IFN-γ⁺) T cell responses measured in PBMC. (FIG. 2E) Plot comparing the Gag-specific T cell responses in macaques immunized twice with low dose (25 μg; described in FIG. 1) and high dose (100 μg) mRNA/LNP vaccines, respectively. Responses in macaques immunized twice with 1 mg gag DNA (grey symbols) are included. The DNA vaccine contained IL-12 DNA as vaccine adjuvant and was administered by IM injection followed by electroporation. The p value is from t test (Mann-Whitney). (FIG. 2F-2G) Analysis of gag mRNA/LNP vaccine induced memory CD4 immune responses. (FIG. 2F) Gating strategy for unstimulated and Gag peptide stimulated memory T cells producing IFN-g and TNFa. (FIG. 2G) Pie charts showing responses of the 4 animals in the high dose vaccine group.

FIG. 3A-3F show changes in plasma cytokines after vaccination with mRNA/LNPs. Plasma cytokine and chemokine levels were measured using the MSD assay on the day of (D1) and days 2, 4 and 8 (D2, D4, and D8) after each vaccination in macaques receiving mRNA/LNPs vaccine. (FIG. 3A, FIG. 3B) Circulating plasma levels of selected analytes for individual animals (grey lines) and median (bold lines) are shown upon the mRNA/LNP vaccinations, administered with low (25 μg, left panels) or high (100 μg, right panels) dose. (FIG. 3A) Molecules involved in IFN pathway, IFNα-2a, IL-15, IP-10/CXCL10, and ITAC/CXCL11. (FIG. 3B) Molecules involved in the IL-17 pathway, IL-23, IL-6, and IL-17F. (FIG. 3C) Decay in the circulating plasma levels of IL-12/23p40 between D1 and D2 for the individual animals upon each mRNA/LNP vaccination after receiving low dose (left panel) or high dose (right panel) mRNA/LNP vaccine. (FIG. 3D) Heatmap depicts log 2 fold changes (log 2 FC) in 35 analytes overtime upon each vaccination (D2_D1; D4_D1; D8_D1). Cytokine levels at D1 before each vaccination are used as baseline. Comparisons were performed between day1 and day 2 (D2), day 4 (D4) and day 8 (D8), respectively, with data for each animal shown under vaccination 1 to 4 as indicated. (FIG. 3E, FIG. 3F) Volcano plots of data shown in panel D depict differentially expressed analytes upon the vaccination 1 (FIG. 3E) and vaccination 4 (FIG. 3F) at day 2 versus day 1. Dots to the right of zero indicate significant upregulation; dots to the left of zero indicate significant downregulation (adjusted p value<0.05 represented by the broken horizontal line).

FIG. 4A-4E show comparison of cytokine and chemokine levels measured in macaques upon low and high dose mRNA/LNP vaccinations. Plasma cytokine and chemokine levels were measured using the MSD assay in macaques after the 1st and 2nd mRNA vaccine doses, administered at low or high mRNA/LNP doses. (FIG. 4A) Heatmap depicts log 2 fold changes in 34 analytes detected at 24 hours (D2_D1) after Vaccination 1 (left) and Vaccination 2 (right). Cytokine levels at D1 before each vaccination are used as baseline. (FIG. 4B, FIG. 4C) Volcano plots of data shown in panel A depict differentially induced changes upon Vaccination 1 (FIG. 4B) and Vaccination 2 (FIG. 4C) between low and high mRNA vaccine doses. Dots to the right of zero indicate analytes significantly more upregulated in animals receiving the high dose vaccine; dots to the left of zero indicate analytes significantly more upregulated in animals receiving the low dose vaccine (adjusted p value<0.05 represented by the broken horizontal line). (FIG. 4D, FIG. 4E) Overtime changes in inflammatory modulators upon mRNA/LNPs vaccination. Circulating plasma levels of (FIG. 4D) IL-17 family of cytokines (IL-17A/F, IL-17B, IL-17C, IL-17D) and (FIG. 4E) IL-1Ra for individual animals (grey lines) and median (bold lines) are shown upon mRNA vaccination, administered at low (left panels) and high (right panels) mRNA/LNP doses.

FIG. 5A-5D show that gag DNA booster vaccination of macaques primed with mRNA/LNP vaccinations increased T cell responses. (FIG. 5A) Schematic representation of the mRNA/LNP prime-DNA boost vaccination regimen. Five animals previously immunized four times (V1-V4) with gag mRNA/LNP (group 2, 25 μg dose, described in FIG. 1) received a single gag DNA vaccination (V5; 2 mg dose) at 10 weeks after the last mRNA/LNP vaccination (V4). A group of naïve macaques (n=5) received a single gag DNA vaccination and was added for comparison. The gag DNA vaccine was administered by IM injection followed by electroporation. (FIG. 5B) Gag Ab titers after the single DNA vaccination were plotted over time. (FIG. 5C-FIG. 5D) Gag-specific cellular analysis was performed by flow cytometry two weeks after the DNA vaccination. (FIG. 5C) Total Gag-specific (CD3+IFN-γ⁺) T cell responses and (FIG. 5D) Gag-specific memory (CD3⁺CD95+IFN-γ⁺) T cell responses are shown. The percent of Gag-specific IFN-γ⁺CD4⁺ (left panel) and CD8⁺ (right panel) memory T cells in blood were plotted. The p values are from t test (Mann-Whitney).

FIG. 6A-6L show that gag mRNA/LNP booster vaccination of macaques with pre-existing Gag T cell immunity increased T cell responses. (FIG. 6A, FIG. 6B) Schematic representations of the DNA prime-mRNA/LNP booster vaccination regimens. (FIG. 6A) The macaques (n=3) in group A previously received 4 HIV gag DNA vaccinations (week 0, 4, 8 and 71). After a rest period of 89 weeks, they received a single gag mRNA/LNP (25 μg) booster vaccination (V5). (FIG. 6B) Animals in group B received a single gag DNA prime (V1; 2 mg dose), followed 15 weeks later by two gag mRNA/LNP booster vaccinations (V2, V3; 25 μg dose) spaced 5 weeks apart. (FIG. 6C, FIG. 6D) Gag-specific Ab endpoint titers (log) were measured by ELISA during the course of the studies. (FIG. 6C) Gag Ab were measured starting 6 weeks before study start (week 154), at the day of vaccination (week 160), and 2 and 4 weeks upon the mRNA/LNP boost. (FIG. 6D) Gag Ab responses were measured after the gag DNA vaccination, at the start and post the mRNA/LNP vaccinations. (FIG. 6E, FIG. 6F) Gag-specific T cell responses measured by flow cytometry at the indicated time points for (FIG. 6E) group A and (FIG. 6F) group B. Grey symbols denote responses after the DNA vaccination, green symbols denote responses after mRNA/LNP vaccination. (FIG. 6G, FIG. 6H) Gag-specific responses in total (CD3+IFN-γ⁺) and memory (CD3⁺CD95+IFN-γ⁺) T cell subsets are shown. Changes in (FIG. 6I) proliferation, measured by Ki67 staining, and (FIG. 6K) cytotoxicity, measured by granzyme B content, are shown for animals from group A. (FIG. 6L) Dot plots (upper panels) from a representative animal (LI19) from group B showing T-bet, granzyme B content and expression of the co-stimulatory immune checkpoint molecule CD137 and the CD69 activation marker among the Gag-specific IFN-γ⁺ memory CD8⁺ T cells after the last vaccination. The graph (lower panel) shows the peak responses after the last vaccination with data from 4 of 5 animals with positive Gag-specific memory (CD8⁺CD95⁺IFN-γ⁺) T cell responses.

FIG. 7A-7B show changes in body temperature upon mRNA/LNP vaccinations. Body temperatures (in Fahrenheit) were measured in macaques on day 1, 2, 4 after each mRNA/LNP vaccination. The mRNA/LNP vaccines were administered as (FIG. 7A) low (n=15) and (FIG. 7B) high dose (n=5) and the data were plotted overtime. The individual animals (grey lines) and median (bold lines) were shown. The tables list the number of animals with at least 1° F. increase and the median temperature change with [IQR].

FIG. 8 shows HIV CE/CE+gag DNA vaccination of rhesus macaques. Macaques were vaccinated with CE/CE+Gag DNA following the same schedule used for the mRNA/LNP vaccination (FIGS. 1, group 3). The DNA vaccine (dose: 2 mg prime, 2+2 mg boost) contained IL-12 DNA as vaccine adjuvant and was administered by IM injection followed by electroporation. Plot shows vaccine-induced Gag Ab measured over time as reciprocal endpoint titers (log). The last time points of blood collection were week 95 and 101, respectively, for 3 animals each and these time points were combined plotted as week 98.

FIG. 9A-9B show differential expression analysis comparing changes after the 2nd and 3rd vaccination. Mean log 2 fold changes (Log 2FC) of cytokine levels are shown comparing levels at day 2 to day 1 for all the 15 animals receiving the mRNA/LNP vaccine. Volcano plots of data shown in FIG. 5D depict differentially expressed analytes upon the vaccination 2 (FIG. 9A) and vaccination 3 (FIG. 9B) at day 2 in comparison to day 1. Dots to the right of zero indicate significant upregulation; dots to the left of zero indicate significant downregulation (adjusted p value <0.05 represented by the broken horizontal line).

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present disclosure.

### DETAILED DESCRIPTION OF THE DISCLOSURE

All publications, patents and patent applications cited herein are hereby expressly incorporated by reference for all purposes. In particular, the disclosures of WO2013131099, WO2016183420, and WO2018078053 are hereby expressly incorporated by reference in their entirety.

Before describing the present disclosure in detail, a number of terms will be defined. Unless otherwise required by context, singular terms shall include pluralities and plural terms shall include the singular. For example, the singular forms “a,” “an,” and “the” include plural referents unless the context clearly dictates otherwise. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives unless otherwise indicated.

In the present disclosure, any concentration range, percentage range, ratio range, or integer range is to be understood to include the value of any integer within the recited range and, when appropriate, fractions thereof (such as one tenth and one hundredth of an integer), unless otherwise indicated.

As used herein, the term “about” means±10% of the indicated range, value, sequence, or structure, unless otherwise indicated.

It is noted that terms like “preferably,” “commonly,” and “typically” are not utilized herein to limit the scope of the claimed subject matter or to imply that certain features are critical, essential, or even important to the structure or function of the claimed subject matter. Rather, these terms are merely intended to highlight alternative or additional features that can or cannot be utilized in a particular embodiment of the present disclosure.

For the purposes of describing and defining the present disclosure it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Unless expressly specified otherwise, the term “comprising” is used in the context of the present disclosure to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

As utilized in accordance with the present disclosure, unless otherwise indicated, all technical and scientific terms shall be understood to have the same meaning as commonly understood by one of ordinary skill in the art.

Methods well known to those skilled in the art can be used to construct genetic expression constructs and recombinant cells according to this disclosure. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory. New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker. In the present disclosure, a “nucleic acid” molecule can include, DNA, cDNA and genomic DNA sequences, RNA, messenger RNA, and synthetic nucleic acid sequences. In some embodiments, the nucleic acid molecules are codon-optimized for expression. Thus, “nucleic acid” also encompasses embodiments in which analogs of DNA and RNA are employed. In some embodiments, the nucleic acid component may comprises one or more RNA molecules, such as viral RNA molecules or mRNA molecules that encode the protein of interest.

This disclosure provides heterologous vaccine regimens combining DNA vaccines with mRNA/LNPs vaccine to induce optimal, effective, and balanced humoral and cellular immunity. Specifically, the inclusion of mRNA-based immunogens following DNA vaccination could be useful in immune therapeutic regimens aiming to treat chronic pathological conditions or to enhance pre-existing immunity.

### DNA Constructs

As used herein, the term “DNA construct” refers to a nucleic acid molecule that when introduced into a mammal, induces the expression of the encoded protein of interest, or portion or fragment thereof, within the mammals, and cause the mammals' immune system to become reactive against the protein of interest (antigen). In certain embodiments the DNA construct is a DNA vaccine in the form of a DNA plasmid. A DNA plasmid is one that includes an encoding sequence of a protein of interest, or portion or fragment thereof, that is capable of being expressed in a mammalian cell, upon the DNA plasmid entering after administration. In certain embodiments, administration can be by injection. In some embodiments, the administration uses electroporation. In some embodiments, the DNA construct encodes a sequence for the protein of interest, or portion or fragment thereof, that elicits an immune response in the target subject. In some embodiments, the one or more DNA constructs are optimized for mammalian expression, which can include one or more of the following: including the addition of a Kozak sequence, codon optimization, and RNA optimization.

The one or more priming and/or boosting doses comprising the DNA construct of this disclosure can be formulated for pharmaceutical administration. While any suitable carrier known to those of ordinary skill in the art may be employed in the pharmaceutical compositions of this disclosure, the type of carrier will vary depending on the mode of administration. For parenteral administration, including intranasal, intradermal, subcutaneous or intramuscular injection or electroporation, the carrier preferably comprises water, saline, and optionally an alcohol, a fat, a polymer, a wax, one or more stabilizing amino acids or a buffer. General formulation technologies are known to those of skill in the art (see, for example, Remington: The Science and Practice of Pharmacy (20th edition), Gennaro, ed., 2000, Lippincott Williams & Wilkins; Injectable Dispersed Systems: Formulation, Processing And Performance, Burgess, ed., 2005, CRC Press; and Pharmaceutical Formulation Development of Peptides and Proteins, Frkjr et al., eds., 2000, Taylor & Francis).

The one or more priming and/or boosting doses can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. For further discussions of nasal administration of AIDS-related vaccines, references are made to the following patents, U.S. Pat. Nos. 5,846,978, 5,663,169, 5,578,597, 5,502,060, 5,476,874, 5,413,999, 5,308,854, 5,192,668, and 5, 187,074.

Naked DNA can be administered in solution (e.g., a phosphate-buffered saline solution) by injection, usually by an intra-arterial, intravenous, subcutaneous or intramuscular route. In general, the dose of a naked nucleic acid composition is from about 10 μg to 10 mg for a typical 70 kilogram patient. Subcutaneous or intramuscular doses for naked nucleic acid (typically DNA encoding a fusion protein) will range from 0.1 mg to 50 mg for a 70 kg patient in generally good health. In certain embodiments, about 1 mg to about 20 mg of DNA is administered (for example, about 1 mg, about 2.5 mg, about 4 mg, about 5 mg, about 6 mg, about 7 mg, about 8 mg, about 9 mg, about 10 mg, about 15 mg, or about 20 mg).

Compositions comprising the one or more DNA constructs can be administered once or multiple limes. For vaccination with a DNA construct, administration is performed more than once, for example, 2, 3, 4, 5, 6, 7, 8, 10, 15, 20 or more times as needed to induce the desired response (e.g., specific antigenic response or proliferation of immune cells). Multiple administrations can be administered, for example, bi-weekly, weekly, bi-monthly, monthly, or more or less often, as needed, for a time period sufficient to achieve the desired response.

The DNA constructs of this disclosure are administered to a mammalian host. The mammalian host usually is a human or a primate. In some embodiments, the mammalian host can be a domestic animal, for example, canine, feline, lagomorpha, rodentia, rattus, hamster, murine. In other embodiment, the mammalian host is an agricultural animal, for example, bovine, ovine, porcine, equine, etc.

The one or more priming and/or boosting doses comprising the DNA construct can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular patient, and the route of administration.

The one or more priming and/or boosting doses comprising the DNA construct are administered to a patient in an amount sufficient to elicit a therapeutic effect, e.g., a CD8+, CD4+, and/or antibody response to the protein of interest encoded by the DNA construct. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

Suitable quantities of the DNA construct (e.g., plasmid or naked DNA) can be about 1 μg to about 200 mg, or about 0.1 to 10 mg, or about 1 to 10 mg, but lower levels such as 1-100 μg can be employed. For example about 1 mg, about 2 mg, about 2.5 mg, about 4 mg, about 5 mg, about 6 mg, about 7 mg, about 8 mg, about 9 mg, about 10 mg, about 15 mg, or about 20 mg. In some embodiments, a DNA vaccine, e.g., naked DNA or polynucleotide in an aqueous carrier, can be injected into tissue, e.g., intramuscularly or intradermally, in amounts of from 10 μl per site to about 1 mL per site. The concentration of polynucleotide in a formulation is usually from about 0.1 μg/mL to about 4 mg/mL.

The one or more priming and/or boosting doses comprising the DNA construct may be delivered in a physiologically compatible solution such as sterile PBS in a volume of, e.g., one ml. The doses may also be lyophilized prior to delivery. As well known to those in the art, the dose may be proportional to the weight of a subject.

The one or more priming and/or boosting doses comprising the DNA construct included in the regimen described herein for inducing an immune response can be administered alone, or can be co-administered or sequentially administered with other immunological, antigenic, vaccine, or therapeutic compositions.

The one or more priming and/or boosting doses comprising the DNA construct may also be administered with other agents to potentiate or broaden the immune response, e.g., IL-15, IL-12, IL-2 or CD40 ligand, which can be administered at specified intervals of time, or continuously administered.

The one or more priming and/or boosting doses comprising the DNA construct can additionally be complexed with other components such as peptides, polypeptides and carbohydrates for delivery. For example, expression vectors, nucleic acid vectors that are not contained within a viral particle, can be complexed to particles or beads that can be administered to an individual, for example, using a vaccine gun.

DNA vaccines can be administered by methods well known in the art as described in Donnelly et al. (Ann. Rep. Immunol. 15:617-648 (1997); Feigner et al. (U.S. Pat. No. 5,580,859, issued December 3. 1996); Feigner (U.S. Pat. No. 5,703,055, issued Dec. 30, 1997); and Carson et al. (U.S. Pat. No. 5,679,647, issued Oct. 21, 1997), each of which is incorporated herein by reference. One skilled in the art would know that the choice of a pharmaceutically acceptable carrier, including a physiologically acceptable compound, depends, for example, on the route of administration of the expression vector.

### RNA Constructs

In some embodiments, the one or more priming and/or boosting doses comprising an RNA construct comprises an mRNA sequence encoding the protein of interest, or portion or fragment thereof (i.e., an antigen or antigenic peptide). In an embodiment, the mRNA sequence is a natural and non-modified mRNA. Within the context of the present disclosure, natural and non-modified mRNA encompasses mRNA generated in vitro, without chemical modifications or changes in the sequence. In certain embodiments, the mRNA can be an artificial mRNA. In the context of the present disclosure, artificial mRNA encompasses mRNA with chemical modifications, sequence modifications or non-natural sequences.

Antigen-providing mRNA: An antigen-providing mRNA may typically be an mRNA, having at least one open reading frame that can be translated by a cell or an organism provided with that mRNA. The product of this translation is a peptide or protein that may act as an antigen, preferably as an immunogen. The product may also be a fusion protein composed of more than one immunogen, e.g., a fusion protein that consist of two or more epitopes, peptides or proteins derived from the same or different virus-proteins, wherein the epitopes, peptides or proteins may be linked by linker sequences.

Artificial mRNA (sequence): An artificial mRNA (sequence) may typically be understood to be an mRNA molecule that does not occur naturally. In other words, an artificial mRNA molecule may be understood as a non-natural mRNA molecule. Such mRNA molecule may be non-natural due to its individual sequence (which does not occur naturally) and/or due to other modifications, e.g., structural modifications of nucleotides which do not occur naturally. Typically, artificial mRNA molecules may be designed and/or generated by genetic engineering methods to correspond to a desired artificial sequence of nucleotides (heterologous sequence). In this context an artificial sequence is usually a sequence that may not occur naturally, i.e., it differs from the wild type sequence by at least one nucleotide. The term “wild type” may be understood as a sequence occurring in nature. Further, the term “artificial nucleic acid molecule” is not restricted to mean “one single molecule” but is, typically, understood to comprise an ensemble of identical molecules. Accordingly, it may relate to a plurality of identical molecules contained in an aliquot.

Variant of a nucleic acid sequence, particularly an mRNA: A variant of a nucleic acid sequence refers to a variant of nucleic acid sequences which forms the basis of a nucleic acid sequence. For example, a variant nucleic acid sequence may exhibit one or more nucleotide deletions, insertions, additions and/or substitutions compared to the nucleic acid sequence from which the variant is derived. Preferably, a variant of a nucleic acid sequence is at least 40%, preferably at least 50%, more preferably at least 60%, more preferably at least 70%, even more preferably at least 80%, even more preferably at least 90%, most preferably at least 95% identical to the nucleic acid sequence the variant is derived from. Preferably, the variant is a functional variant. A “variant” of a nucleic acid sequence may have at least 70%, 75%, 80%, 85%, 90%, 95%, 98% or 99% nucleotide identity over a stretch of 10, 20, 30, 50, 75 or 100 nucleotide of such nucleic acid sequence.

Stabilized nucleic acid, preferably mRNA: A stabilized nucleic acid, preferably mRNA typically, exhibits a modification increasing resistance to in vivo degradation (e.g., degradation by an exo- or endo-nuclease) and/or ex vivo degradation (e.g., by the manufacturing process prior to vaccine administration, e.g., in the course of the preparation of the vaccine solution to be administered). Stabilization of RNA can, e.g., be achieved by providing a 5′-CAP-Structure, a Poly-A-Tail, or any other UTR-modification. It can also be achieved by chemical modification or modification of the G/C-content of the nucleic acid. Various other methods are known in the art and conceivable.

In some embodiments, the mRNA does not comprise nucleoside modifications, in particular no base modifications. In a further embodiment, the mRNA does not comprise 1-methylpseudouridine modifications. In one embodiment, the mRNA comprises only the naturally existing nucleosides. In a further embodiment, the mRNA does not comprise any chemical modification and optionally comprises sequence modifications. In a further embodiment, the mRNA only comprises the naturally existing nucleosides adenine, uracil, guanine and cytosine.

Suitable quantities of the RNA construct (mRNA) can be about 1 μg to about 100 μg, or about 25 μg to 100 μg, but lower levels such as 1-25 μg can be employed. For example, about 1 μg, about 2.5 μg, about 4 μg, about 5 μg, about 6 μg, about 7 μg, about 8 μg, about 9 μg, about 10 μg, about 15 μg, about 20 μg, about 25 μg, about 30 μg, about 40 μg, about 50 μg, about 60 μg, about 70 μg, about 80 μg, about 90 μg, or about 100 μg. In certain embodiments, an RNA construct as part of a lipid nanoparticle, can be injected into tissue, e.g., intramuscularly or intradermally, in amounts of from 10 μl per site to about 1 mL per site.

The RNA constructs of this disclosure are administered to a mammalian host. The mammalian host usually is a human or a primate. In some embodiments, the mammalian host can be a domestic animal, for example, canine, feline, lagomorpha, rodentia, rattus, hamster, murine. In other embodiment, the mammalian host is an agricultural animal, for example, bovine, ovine, porcine, equine, etc.

In certain embodiments, this disclosure relates to mRNA formulated with lipid nanoparticles (LNP). In some embodiments, the lipid nanoparticles comprise at least (i) a cationic lipid and/or a PEG-lipid as defined herein; and the RNA construct comprising an mRNA sequence encoding the protein of interest.

### LNP Formulations

The term “lipid nanoparticle”, also referred to as LNP, refers to a particle having at least one dimension on the order of nanometers (e.g., 1-1,000 nm) which includes one or more lipids. In some embodiments, such lipid nanoparticles comprise a cationic lipid and one or more excipient selected from neutral lipids, charged lipids, steroids and polymer conjugated lipids (e.g., a pegylated lipid such as a pegylated lipid). In some embodiments, the mRNA, or a portion thereof, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g., an adverse immune response. In some embodiments, the mRNA or a portion thereof is associated with the lipid nanoparticles.

Lipid nanoparticles, cationic lipids and polymer conjugated lipids (PEG-lipid) were prepared and tested according to the general procedures described in PCT Pub. Nos. WO 2015/199952, WO 2017/004143, WO 2017/075531 and WO2018078053, the full disclosures of which are incorporated herein by reference in their entirety. Lipid nanoparticle (LNP)-formulated mRNA can be prepared using an ionizable amino lipid (cationic lipid), phospholipid, cholesterol and a PEGylated lipid. LNPs were prepared as follows. Cationic lipid, DSPC, cholesterol and PEG-lipid were solubilized in ethanol at a molar ratio of approximately 50:10:38.5:1.5 or 47.5:10:40.8:1.7. Lipid nanoparticles (LNP) comprising compound III-3 were prepared at a ratio of mRNA to Total Lipid of 0.03-0.04 w/w. Briefly, the mRNA was diluted to 0.05 to 0.2 mg/ml in 10 to 50 mM citrate buffer, pH 4. Syringe pumps were used to mix the ethanolic lipid solution with the mRNA aqueous solution at a ratio of about 1:5 to 1:3 (vol/vol) with total flow rates above 15 mL/min. The ethanol was then removed and the external buffer replaced with PBS by dialysis. Finally, the lipid nanoparticles were filtered through a 0.2 μm pore sterile filter. Lipid nanoparticle particle diameter size was 60-90 nm as determined by quasi-elastic light scattering using a Malvern Zetasizer Nano (Malvern, UK). For other cationic lipid compounds mentioned in the present specification, the formulation process is similar.

Lipid nanoparticles are not restricted to any particular morphology, and should be interpreted as to include any morphology generated when a cationic lipid and optionally one or more further lipids are combined, e.g. in an aqueous environment and/or in the presence of a nucleic acid compound. For example, a liposome, a lipid complex, a lipoplex and the like are within the scope of a lipid nanoparticle.

In some embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the mRNA, when present in the lipid nanoparticles, is resistant in aqueous solution to degradation with a nuclease. As used herein, the mean diameter may be represented by the z-average as determined by dynamic light scattering.

In some embodiments, a LNP may comprise any lipid capable of forming a particle to which the one or more nucleic acid molecules are attached and/or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In certain embodiments, the mRNA-comprising LNP comprises one or more cationic lipids as defined herein, and one or more stabilizing lipids. Stabilizing lipids include neutral lipids and pegylated lipids.

In some embodiments, the LNP comprises a cationic lipid. The cationic lipid is preferably cationisable, i.e., it becomes protonated as the pH is lowered below the pKa of the ionizable group of the lipid, but is progressively more neutral at higher pH values. When positively charged, the lipid is then able to associate with negatively charged nucleic acids. In certain embodiments, the cationic lipid comprises a zwitterionic lipid that assumes a positive charge on pH decrease. The LNP may comprise any lipid capable of forming a particle to which the one or more nucleic acid molecules are attached, or in which the one or more nucleic acid molecules are encapsulated.

In certain embodiments, the LNP may comprise any further cationic or cationisable lipid, i.e., any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy) propyl)-N,N,N-trimethylammonium chloride (DOTMA); N, N-distearyl-N, N-dimethylammonium bromide (DDAB); N-(2,3dioleoyloxy) propyl)-N,N, N-trimethylammonium chloride (DOTAP); 3-(N—(N′, N′dimethylaminoethane)-carbamoyl) cholesterol (DC-Chol), N-(1-(2,3-dioleoyloxy) propyl)N-2-(sperminecarboxamido) ethyl)-N,N-dimethyl-ammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy) propylamine (DODMA), and N-(1,2dimyristyloxyprop-3-yl)-N,N-dimethyl-N-hydroxyethyl ammonium bromide (DMRIE).

In some embodiments, a number of commercial preparations of cationic lipids are available which can be used in the LNPs disclosed herein. These can include, for example, LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy) propyl)-N-(2-(sperminecarboxamido) ethyl)-N,N-dimethyl-ammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In an embodiment, the further cationic lipid is an amino lipid. Suitable amino lipids useful in the disclosure include those described in WO2012/016184, incorporated herein by reference in its entirety. Representative amino lipids include, but are not limited to, 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

In some embodiments, the amount of the permanently cationic lipid or lipidoid should also be selected taking the amount of the nucleic acid cargo into account. In certain embodiments, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the nucleic acid which is used as cargo. The N/P ratio may be calculated on the basis that, for example, 1 pg RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups. Such low N/P ratios are commonly believed to be detrimental to the performance and in vivo efficacy of such carrier-cargo complexes, or nucleic-acid loaded nanoparticles. However, such N/P ratios are indeed useful in the context of the present disclosure, in particular when the local or extravascular administration of the nanoparticles is intended. Here, the respectively nanoparticles have been found to be efficacious and at the same time well-tolerated.

In certain embodiments, the LNP comprises one or more additional lipids which stabilize the formation of particles during their formation. Suitable stabilizing lipids can include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, cephalins, and cerebrosides. Exemplary neutral lipids can include, but are not limited to, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-Icarboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP can comprise an additional, stabilizing-lipid which is a polyethylene glycol-lipid (pegylated lipid). Suitable polyethylene glycollipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly (ethylene glycol) 2000) carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(omega-methoxy(polyethoxy)ethyl)butanedioate (PEG-S-DMG), a pegylated ceramide (PEG-cer), or a PEG dialkoxypropylcarbamate such as omega-methoxy(polyethoxy)ethyl-N-(2,3di(tetradecanoxy)propyl)carbamate or 2,3-di(tetradecanoxy)propyl-N-(omega-methoxy(polyethoxy)ethyl)carbamate. In various embodiments, the molar ratio of the cationic lipid to the pegylated lipid ranges from about 100:1 to about 25:1.

In certain embodiments, the PEG lipid is present in the LNP in an amount from about 1 to about 10 mole percent, relative to the total lipid content of the nanoparticle. In an embodiment, the PEG lipid is present in the LNP in an amount from about 1 to about 5 mole percent. In another embodiment, the PEG lipid is present in the LNP in about 1 mole percent or about 1.5 mole percent.

In certain embodiments, the LNP comprises one or more targeting moieties which are capable of targeting the LNP to a cell or cell population. For example, in an embodiment, the targeting moiety is a ligand which directs the LNP to a receptor found on a cell surface.

In certain embodiments, the LNP comprises one or more internalization domains. For example, in an embodiment, the LNP comprises one or more domains which bind to a cell to induce the internalization of the LNP. For example, in one embodiment, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Additional exemplary LNPs and their manufacture are described in the art, for example in U.S. Patent Application Publication No. US20120276209, WO2019077053, Semple et al., 2010, Nat Biotechnol., 28 (2): 172-176; Akinc et al., 2010, Mol Ther., 18 (7): 1357-1364; Basha et al., 2011, Mol Ther, 19 (12): 2186-2200; Leung et al., 2012, J Phys Chem C Nanomater Interfaces, 116 (34): 18440-18450; Lee et al., 2012, Int J Cancer., 131 (5): E781-90; Belliveau et al., 2012, Mol Ther nucleic Acids, 1: e37; Jayaraman et al., 2012, Angew Chem Int Ed Engl., 51 (34): 8529-8533; Mui et al., 2013, Mol Ther Nucleic Acids. 2, e139; Maier et al., 2013, Mol Ther., 21 (8): 1570-1578; and Tam et al., 2013, Nanomedicine, 9 (5): 665-74, each of which are incorporated by reference in their entirety.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. As mentioned, the mean diameter may correspond to the z-average as determined by dynamic light scattering.

In some preferred embodiments, the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In certain embodiments, this disclosure further relates to pharmaceutical compositions comprising at least one lipid nanoparticle comprising an RNA construct comprising an mRNA sequence encoding at least one peptide of interest or antigenic protein as disclosed herein. In an embodiment, the mRNA sequence encodes at least one peptide of interest or antigenic protein. In an alternative embodiment, the mRNA sequence encodes more than one peptide of interest or antigenic protein.

In some embodiments, the pharmaceutical compositions can comprise a lipid nanoparticle as disclosed herein, wherein the lipid nanoparticle comprises more than one RNA construct, which each RNA construct comprises a different mRNA sequence encoding a peptide of interest or antigenic protein.

In some embodiments, the pharmaceutical compositions can comprise a second lipid nanoparticle, wherein the RNA construct comprised by the second lipid nanoparticle is different from the RNA construct comprised by the first lipid nanoparticle.

In some embodiments, the pharmaceutical compositions are provided as a vaccine. As used herein, a vaccine is typically understood to be a prophylactic or therapeutic material providing at least one antigen or antigenic function. The antigen or antigenic function may stimulate the body's adaptive immune system to provide an adaptive immune response.

### Adjuvants

In some embodiments, the one or priming and/or the one or more boosting doses comprising either a DNA construct of an RNA construct can also comprise suitable pharmaceutically acceptable adjuvants and/or excipients. In some embodiments, the adjuvant is added in order to enhance the immunostimulatory properties of the one or more priming doses and/or one or more boosting doses.

As used herein, the term “adjuvant” can refer to any compound, which is suitable to support administration and delivery of the one or more priming doses and/or one or more boosting doses (DNA and/or RNA vaccines) as disclosed herein. Furthermore, such an adjuvant may, without being bound thereto, initiate or increase an immune response of the innate immune system, i.e., a non-specific immune response. In other words, when administered, the one or more priming doses and/or one or more boosting doses (DNA and/or RNA vaccines) as disclosed herein typically initiates an adaptive immune response due to an antigen as defined herein or a fragment or variant thereof, which is encoded by the DNA construct and/or the RNA construct contained the one or more priming doses and/or one or more boosting doses (DNA and/or RNA vaccines) as disclosed herein. Additionally, the one or more priming doses and/or one or more boosting doses (DNA and/or RNA vaccines) as disclosed herein may generate an (supportive) innate immune response due to addition of an adjuvant as defined herein. In certain embodiments, the term “adjuvant” can be understood not to comprise agents which confer immunity by themselves. An adjuvant assists the immune system unspecifically to enhance the antigen-specific immune response by, e.g., promoting presentation of an antigen to the immune system or induction of an unspecific innate immune response. Furthermore, an adjuvant may preferably, e.g., modulate the antigen-specific immune response by, e.g., shifting the dominating Th2-based antigen specific response to a more Th1-based antigen specific response or vice versa. Accordingly, an adjuvant may favorably modulate cytokine expression/secretion, antigen presentation, type of immune response etc.

In some embodiments, an adjuvant may be selected from any adjuvant known to a skilled person and suitable for the present case, i.e., supporting the induction of an immune response in a mammal. For example, an adjuvant may be selected from the group consisting of, without being limited thereto, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydroacetate); CALCITRIOL™ (1-alpha,25-dihydroxy-vitamin D3); calcium phosphate gel; CAP™(calcium phosphate nanoparticles); cholera holotoxin, cholera-toxin-A1-protein-A-D-fragment fusion protein, subunit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methypropyl)-1H-imidazo[4,5-c]quinoline-4-amine): ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1beta; interleukin-2; interleukin-7; interleukin-12; ISCOMS™; ISCOPREP 7.0.3™; liposomes; LOXORIBINE™ (7-allyl-8-oxoguanosine); LT oral adjuvant (E. coli labile enterotoxin-protoxin); microspheres and microparticles of any composition; MF59™; (squalene-water emulsion); MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and D-MURAPALMITINE™ (Nac-Mur-L-Thr-D-isoGln-sn-glyceroldipalmitoyl); NAGO (neuraminidase-galactose oxidase); nanospheres or nanoparticles of any composition; NISVs (non-ionic surfactant vesicles); PLEURANT (P3-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.): STIMULON™ (QS-21); Quil-A (Quil-A saponin); S-28463 (4-amino-otec-dimethyl-2-ethoxymethyl-1H-imidazo[4,5c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypro-pylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin; microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide.

In certain embodiments, an adjuvant may be selected from adjuvants, which support induction of a Th1-immune response or maturation of naive T-cells, such as GM-CSF, IL-12, IFN-gamma, any immunostimulatory nucleic acid as defined above, preferably an immunostimulatory RNA and/or CpG DNA. In some embodiments, it is also possible that the compositions disclosed herein contain, besides the antigen-providing RNA, further components which are selected from the group consisting of: further antigens (e.g., in the form of a peptide or protein) or further antigen-encoding nucleic acids; a further immunotherapeutic agent; one or more auxiliary substances; or any further compound, which is known to be immunostimulating due to its binding affinity (as ligands) to human Toll-like receptors; and/or an adjuvant nucleic acid, preferably an immunostimulatory RNA (isRNA).

In certain embodiments, the one or more priming doses and/or one or more boosting doses (DNA and/or RNA vaccines) as disclosed herein can additionally contain one or more auxiliary substances in order to increase its immunogenicity or immunostimulatory capacity. A synergistic action of the mRNA as defined herein and of an auxiliary substance can be achieved. Depending on the various types of auxiliary substances, various mechanisms can come into consideration in this respect. For example, compounds that permit the maturation of dendritic cells (DCs), for example lipopolysaccharides, TNF-alpha or CD40 ligand, form a first class of suitable auxiliary substances. In general, it is possible to use as auxiliary substance any agent that influences the immune system in the manner of a “danger signal” (LPS, GP96, etc.) or cytokines, such as GM-CFS, which allow an immune response to be enhanced and/or influenced in a targeted manner. Examples of auxiliary substances can include, but is not limited to, cytokines, such as monokines, lymphokines, interleukins or chemokines, that further promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta or TNF-alpha, growth factors, such as hGH.

Suitable adjuvants may furthermore be selected from nucleic acids having the formula GIXmGn, wherein: G is guanosine, uracil or an analogue of guanosine or uracil; X is guanosine, uracil, adenosine, thymidine, cytosine or an analogue of the above-mentioned nucleotides; I is an integer from 1 to 40, wherein when I=1 G is guanosine or an analogue thereof, when I>1 at least 50% of the nucleotides are guanosine or an analogue thereof; m is an integer and is at least 3; wherein when m=3 X is uracil or an analogue thereof, when m>3 at least 3 successive uracils or analogues of uracil occur; n is an integer from 1 to 40, wherein when n=1 G is guanosine or an analogue thereof, when n >1 at least 50% of the nucleotides are guanosine or an analogue thereof, or formula: (NuGIXmGnNv) a, wherein: G is guanosine (guanine), uridine (uracil) or an analogue of guanosine (guanine) or uridine (uracil), preferably guanosine (guanine) or an analogue thereof; X is guanosine (guanine), uridine (uracil), adenosine (adenine), thymidine (thymine), cytidine (cytosine), or an analogue of these nucleotides (nucleosides), preferably uridine (uracil) or an analogue thereof; N is a nucleic acid sequence having a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides); a is an integer from 1 to 20, preferably from 1 to 15, most preferably from 1 to 10; I is an integer from 1 to 40, wherein when I=1, G is guanosine (guanine) or an analogue thereof, when I>1, at least 50% of these nucleotides (nucleosides) are guanosine (guanine) or an analogue thereof; m is an integer and is at least 3; wherein when m=3, X is uridine (uracil) or an analogue thereof, and when m>3, at least 3 successive uridines (uracils) or analogues of uridine (uracil) occur; n is an integer from 1 to 40, wherein when n=1, G is guanosine (guanine) or an analogue thereof, when n >1, at least 50% of these nucleotides (nucleosides) are guanosine (guanine) or an analogue thereof; u,v may be independently from each other an integer from 0 to 50, preferably wherein when u=0, v>1, or when v=0, u >1; wherein the nucleic acid molecule of formula (NuGIXmGnNv) a has a length of at least 50 nucleotides, preferably of at least 100 nucleotides, more preferably of at least 150 nucleotides, even more preferably of at least 200 nucleotides and most preferably of at least 250 nucleotides.

Other suitable adjuvants may furthermore be selected from nucleic acids having the formula: CIXmCn, wherein: C is cytosine, uracil or an analogue of cytosine or uracil; X is guanosine, uracil, adenosine, thymidine, cytosine or an analogue of the above-mentioned nucleotides; I is an integer from 1 to 40, wherein when I=1 C is cytosine or an analogue thereof, when I >1 at least 50% of the nucleotides are cytosine or an analogue thereof; m is an integer and is at least 3; wherein when m=3 X is uracil or an analogue thereof, when m>3 at least 3 successive uracils or analogues of uracil occur; n is an integer from 1 to 40, wherein when n=1 C is cytosine or an analogue thereof, when n >1 at least 50% of the nucleotides are cytosine or an analogue thereof. In this context, the disclosure of WO2008/014979 and WO2009/095226 is also incorporated herein by reference.

In some embodiments, the one or more priming doses and/or the one or more boosting doses may also be administered with other agents to potentiate or broaden the immune response, e.g., IL-15, IL-12, IL-2, IL-7, or CD40 ligand, which can be administered at specified intervals of time, or continuously administered. In some embodiments, the one or more priming doses and/or the one or more boosting doses may also be administered with 0.1 to 20 μg/kg of IL-15, IL-12, IL-2, IL-7, or CD40 ligand.

### Vaccination Protocols

The vaccination protocol for the one or more priming doses and/or the one or more boosting doses for the immunization of a subject against the protein of interest (or a combination of at least two, three, four, five, six, seven, eight, nine, ten, eleven, twelve or more proteins of interest), as defined herein typically comprises a series of single doses or dosages of the DNA construct and the lipid nanoparticle (LNP) comprising an RNA construct as disclosed herein.

In some embodiments, one or more priming doses refers to the immunization of a subject against the protein of interest (or a combination of at least two, three, four, five, six, seven, eight, nine, ten, eleven, twelve or more proteins of interest), comprises a series of single doses of the DNA construct. In certain embodiments, the one or more priming doses comprises two, three, four, or five doses, each separated by at least about 2 weeks, 3 weeks, 4 weeks, 5 weeks, 6 weeks, 7 weeks, 8 weeks, or more. For example, in an embodiment, a total of three priming doses comprising a DNA construct can be administered, the first priming dose followed by a second priming dose after about 4 weeks, and the third priming dose about 8 weeks after the first. For example, in another embodiment, a total of four priming doses comprising a DNA construct can be administered, the first priming dose followed by a second priming dose after about 8 weeks, and the third priming dose about 16 weeks after the first priming dose, and the fourth priming dose about 10 months after the first priming dose. For example, in yet another embodiment, a total of three priming doses comprising a DNA construct can be administered, at Day 0, at about Day 28 and at about Day 84.

In some embodiments, one or more boosting doses refers to the immunization of a subject against the protein of interest (or a combination of at least two, three, four, five, six, seven, eight, nine, ten, eleven, twelve or more proteins of interest), comprises a series of single doses comprising a lipid nanoparticle (LNP) comprising an RNA construct encoding the protein of interest. In certain embodiments, the one or more boosting doses comprises two, three, four, or five doses, each separated by at least about 2 weeks, 3 weeks, 4 weeks, 5 weeks, 6 weeks, 7 weeks, 8 weeks, or more. For example, in an embodiment, a total of three boosting doses comprising an RNA construct can be administered, the first boosting dose followed by a second boosting dose after about 4 weeks, and the third boosting dose about 8 weeks after the first. For example, in another embodiment, a total of four boosting doses comprising an RNA construct can be administered, the first boosting dose followed by a second boosting dose after about 8 weeks, and the third boosting dose about 16 weeks after the first boosting dose, and the fourth boosting dose about 10 months after the first boosting dose. For example, in yet another embodiment, a total of three boosting doses comprising an RNA construct can be administered, at Day 0, at about Day 28 and at about Day 84.

In certain embodiments, the time between the one or more priming doses and the one or more boosting doses can be about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In some embodiments, the time between the one or more priming doses and the one or more boosting doses can be about 3 months, about 4 months, about 5 months, about 6 months, about 7 months, about 8 months, about 9 months, about 10 months, about 11 months, about 12 months, about 18 months, or about 24 months.

In this context, each single dosage preferably comprises the administration of the same protein of interest, or antigen or the same combination of antigens as defined herein, wherein the interval between the administration of two single dosages can vary from at least one day, preferably 2, 3, 4, 5, 6 or 7 days, to at least one week, preferably 2, 3, 4, 5, 6, 7 or 8 weeks. The intervals between single dosages may be constant or vary over the course of the immunization protocol, e.g., the intervals may be shorter in the beginning and longer towards the end of the protocol. Depending on the total number of single dosages and the interval between single dosages, the immunization protocol may extend over a period of time, which preferably lasts at least one week, more preferably several weeks (e.g., 2, 3, 4, 5, 6, 7, 8, 9, 10, 11 or 12 weeks), even more preferably several months (e.g., 3, 4, 5, 6, 7, 8, 9, 10, 11, 12, 18 or 24 months). In some embodiments, a single dosage encompasses the administration of a protein of interest. In some embodiments, the one or more priming doses and/or the one or more boosting doses comprise a combination of at least two, three, four, five, six, seven, eight, nine, ten, eleven, twelve or more proteins of interest (for example, comprising two, three, four, five, six, seven, eight, nine, ten, eleven, or twelve conserved elements of a protein of interest) as defined herein and may therefore involve at least one, preferably 1, 2, 3, 4, 5, 6, 7, 8, 9, 10, 11 or 12 injections. In some embodiments, the priming dose can be administered as a single dosage typically in one injection. In certain embodiments, the one or more boosting doses comprising an LNP comprising an RNA construct comprises separate mRNA formulations encoding distinct antigens as defined herein, the minimum number of injections carried out during the administration of a single dosage corresponds to the number of separate components of the vaccine. In certain embodiments, the administration of a single dosage may encompass more than one injection for each component of the vaccine (e.g., a specific mRNA formulation comprising an mRNA encoding, for instance, one antigenic peptide or protein as defined herein). For example, parts of the total volume of an individual component of the vaccine may be injected into different body parts, thus involving more than one injection. In a more specific example, a single dosage of a vaccine comprising four separate mRNA formulations, each of which is administered in two different body parts, comprises eight injections. Typically, a single dosage comprises all injections required to administer all components of the vaccine, wherein a single component may be involve more than one injection as outlined above. In the case, where the administration of a single dosage of the vaccine encompasses more than one injection, the injection are carried out essentially simultaneously or concurrently, i.e., typically in a time-staggered fashion within the time-frame that is required for the practitioner to carry out the single injection steps, one after the other. The administration of a single dosage therefore can extend over a time period of several minutes, e.g., 2, 3, 4, 5, 10, 15, 30 or 60 minutes.

### Proteins of Interest (Antigens)

As disclosed herein, the terms “protein of interest” or “antigen” can refer to proteins, protein fragments or peptides derived from pathogenic organisms, in particular bacterial, viral or protozoological (multicellular) pathogenic organisms, which evoke an immunological reaction by a subject, for example, a mammalian subject or human subject. In certain embodiments, a protein of interest is a surface antigen, e.g., proteins (or fragments of proteins, e.g., the exterior portion of a surface antigen) located at the surface of the virus or the bacterial or protozoological organism. Antigens may be recognized by the immune system, preferably by the adaptive immune system, and are capable of triggering an antigen-specific immune response, e.g., by formation of antibodies and/or antigen-specific T cells as part of an adaptive immune response. Typically, an antigen may be or may comprise a peptide or protein which may be presented by the MHC to T-cells. In certain embodiments of the present disclosure, a protein of interest and/or an antigen may be the product of translation of a provided nucleic acid molecule, via the DNA construct and/or the RNA construct as defined herein. In this context, also conserved elements, fragments, variants and derivatives of peptides and proteins comprising at least one epitope are understood as antigen.

As used herein, the terms “epitope” or “antigen determinant” can refer to T cell epitopes or parts of the protein of interest in the context of the present disclosure, and may comprise fragments preferably having a length of about 6 to about 20 or even more amino acids, e.g., fragments as processed and presented by MHC class I molecules, preferably having a length of about 8 to about 10 amino acids, e.g., 8, 9, or 10, (or even 11, or 12 amino acids), or fragments as processed and presented by MHC class II molecules, preferably having a length of about 13 or more amino acids, e.g., 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule.

In certain embodiments, the protein of interest can be derived from a pathogen associated with infectious disease which are selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV). Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104: H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBOV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, the protein of interest can be an antigen (antigen derived from a pathogen associated with infectious disease) selected from the following antigens: Outer membrane protein A OmpA, biofilm associated protein Bap, transport protein Muck (Acinetobacter baumannii, Acinetobacter infections)); variable surface glycoprotein VSG, microtubule-associated protein MAPP15, trans-sialidase TSA (Trypanosoma brucei, African sleeping sickness (African trypanosomiasis)); HIV p24 antigen, HIV envelope proteins (Gp120, Gp41, Gp160), polyprotein GAG, negative factor protein Nef, trans-activator of transcription Tat (HIV (Human immunodeficiency virus; or any HIV antigen sequence as disclosed in WO2013131099 and WO2016183420, which are incorporated by reference herein in their entirety), AIDS (Acquired immunodeficiency syndrome)); galactose-inhibitable adherence protein GIAP, 29 kDa antigen Eh29, Gal/GalNAc lectin, protein CRT, 125 kDa immunodominant antigen, protein M17, adhesin ADH112, protein STIRP (Entamoeba histolytica, Amoebiasis); Major surface proteins 1-5 (MSP1a, MSP1b, MSP2, MSP3, MSP4, MSP5), type IV secreotion system proteins (VirB2, VirB7, VirB11, VirD4) (Anaplasma genus, Anaplasmosis); protective Antigen PA, edema factor EF, lethal factor LF, the S-layer homology proteins SLH (Bacillus anthracis, Anthrax); acranolysin, phospholipase D, collagen-binding protein CbpA (Arcanobacterium haemolyticum, Arcanobacterium haemolyticum infection); nucleocapsid protein NP, glycoprotein precursor GPC, glycoprotein GP1, glycoprotein GP2 (Junin virus, Argentine hemorrhagic fever); chitin-protein layer proteins, 14 kDa surface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp v13, allergen Asp f3, major conidial surface protein rodlet A, protease Pep1p, GPI-anchored protein Gel1p, GPI-anchored protein Crf1p (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (a1, a2, b, c), 12D3, 11C5, 21B4, P29, variant erythrocyte surface antigen VESA1, Apical Membrane Antigen 1 AMA-1 (Babesia genus, Babesiosis); hemolysin, enterotoxin C, PXO1-51, glycolate oxidase, ABC-transporter, penicillin-bingdn protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocystis hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin binding protein A DbpA, decorin binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein visE (Borrelia genus, Borrelia infection); Botulinum neurotoxins BONT/A1, BONT/A2, BONT/A3, BONT/B, BONT/C, BONT/D, BONT/E, BONT/F, BONT/G, recombinant botulinum toxin F He domain FHc (Clostridium botulinum, Botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein RpIL, OmpA-like transmembrane domain-containing protein Omp31, immunogenic 39-kDa protein M5 P39, zinc ABC transporter periplasmic zinc-binding protein znuA, periplasmic immunogenic protein Bp26, 30S ribosomal protein S12 RpsL, glyceraldehyde-3-phosphate dehydrogenase Gap, 25 kDa outer-membrane immunogenic protein precursor Omp25, invasion protein B lalB, trigger factor Tig, molecular chaperone Dnak, putative peptidyl-prolyl cis-trans isomerase SurA, lipoprotein Omp19, outer membrane protein MotY Omp16, conserved outer membrane protein D15, malate dehydrogenase Mdh, component of the Type-IV secretion system (T4SS) VirJ, lipoprotein of unknown function BAB1_0187 (Brucella genus, Brucellosis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, genome polyprotein, Sapoviurus capsid protein VP1, protein Vp3, geome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein Peb1A, protein FspA1, protein FspA2 (Campylobacter genus, Campylobacteriosis); glycolytic enzyme enolase, secreted aspartyl proteinases SAP1-10, glycophosphatidylinositol (GPI)-linked cell wall protein, protein Hyr1, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LalB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Par1, mucin-Associated Surface Proteins MPSP (Trypanosoma cruzi, Chagas Disease (American trypanosomiasis)); envelope glycoproteins (gB, gC, gE, gH, gI, gK, gL) (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis ptrotein F TcpF, cholera enterotoxin subunit A, cholera enterotoxin subunit B, Heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwp13, cysteine protease Cwp19, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: sprike proteins S, envelope proteins E, membrane proteins M, nucleocapsid proteins N (usually rhinoviruses and coronaviruses, Common cold (Acute viral rhinopharyngitis; Acute coryza)); prion protein Prp (CJD prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GalXM, glucuronoxylomannan GXM, mannoprotein MP (Cryptococcus neoformans, Cryptococcosis); acidic ribosomal protein P2 CpP2, mucin antigens Muc1, Muc2, Muc3 Muc4, Muc5, Muc6, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gp15, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein pp15, capsid-proximal tegument protein pp150, protein M45, DNA polymerase UL54, helicase UL 105, glycoprotein gM, glycoprotein gN, glcoprotein H, glycoprotein B gB, protein UL83, protein UL94, protein UL99 (Cytomegalovirus (CMV), Cytomegalovirus infection); capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoeba fragilis, Dientamoebiasis); diphtheria toxin precursor Tox, diphteria toxin DT, pilin-specific sortase SrtA, shaft pilin protein SpaA, tip pilin protein SpaC, minor pilin protein SpaB, surface-associated protein DIP1281 (Corynebacterium diphtheriae, Diphtheria); glycoprotein GP, nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vCOD prion, Variant Creutzfeldt-Jakob disease (vCOD, nvCOD)); UvrABC system protein B, protein Flp1, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspA1, protein LspA2, protein LspB, outer membrane component DsrA, lectin DitA, lipoprotein Hip, major outer membrane protein OMP, outer membrane protein OmpA2 (Haemophilus ducreyi, Chancroid); aspartyl protease 1 Pep1, phospholipase B PLB, alpha-mannosidase 1 AMN1, glucanosyltransferase GEL1, urease URE, peroxisomal matrix protein Pmp1, proline-rich antigen Pra, humal T-cell reactive protein TcrP (Coccidioides immitis and Coccidioides posadasii, Coccidioidomycosis); allergen Tri r 2, heat shock protein 60 Hsp60, fungal actin Act, antigen Tri r2, antigen Tri r4, antigen Tri t1, protein IV, glycerol-3-phosphate dehydrogenase Gpd1, osmosensor HwSho1A, osmosensor HwSho1B, histidine kinase HwHhk7B, allergen Mala s 1, allergen Mala s 11, thioredoxin Trx Mala s 13, allergen Mala f, allergen Mala s (usually Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 MSP2, major surface protein 4 MSP4, MSP variant SGV1, MSP variant SGV2, outer membrane protein OMP, outer membrane protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins SalA and SalB, collagen adhesin Scm, surface proteins Fms1 (EbpA (fm), Fms5 (EbpB (fm), Fms9 (EpbC (fm) and Fms10, protein EbpC (fm), 96 kDa immunoprotective glycoprotein G1 (Enterococcus genus, Enterococcus infection); genome polyprotein, polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adr1 (RP827), cell surface protein Adr2 (RP828), cell surface protein SCA1, Invasion protein invA, cell division protein fts, secretion proteins see 0family, virulence proteins virB, tlyA, tlyC, parvulin-like protein Pip, preprotein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein H, glycoprotein gp350, glycoprotein gp110, glycoprotein gp42, glycoprotein gHgL, glycoprotein gB (Epstein-Barr Virus (EBV), Epstein-Barr Virus Infectious Mononucleosis); capsid protein VP2, capsid protein VP1, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins L1 and L2, Kunitz-type protein K™, leucine aminopeptidase LAP, cysteine proteinase Fas2, saposin-like protein-2 SAP-2, thioredoxin peroxidases TPx, Prx-1, Prx-2, cathepsin I cysteine proteinase CL3, protease cathepsin L CL1, phosphoglycerate kinase PGK, 27-kDa secretory protein, 60 kDa protein HSP35alpha, glutathione transferase GST, 28.5 kDa tegumental antigen 28.5 kDa TA, cathepsin B3 protease CatB3, Type I cystatin stefin-1, cathepsin L5, cathepsin Llg and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, lota toxin component Ib, protein CPE1281, pyruvate ferredoxin oxidoreductase, elongation factor G EF-G, perfringolysin O Pfo, glyceraldehyde-3-phosphate dehydrogenase GapC, Fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); phospholipase C PLC, heat-labile enterotoxin B, lota toxin component Ib, protein CPE1281, pyruvate ferredoxin oxidoreductase, elongation factor G EF-G, perfringolysin O Pfo, glyceraldehyde-3-phosphate dehydrogenase GapC, fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (usually Clostridium perfringens; other Clostridium species, Gas gangrene (Clostridial myonecrosis)); lipase A, lipase B, peroxidase Dec1 (Geotrichum candidum, Geotrichosis); prion protein (GSS prion, Gerstmann-Straussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, alpha-giardin, alpha8-giardin, alpha1-guiardin, beta-giardin, cystein proteases, glutathione-S-transferase GST, arginine deiminase ADI, fructose-1,6-bisphosphat aldolase FBA, Giardia trophozoite antigens GTA (GTA1, GTA2), ornithine carboxyl transferase OCT, striated fiber-asseblin-like protein SALP, uridine phosphoryl-like protein UPL, alpha-tubulin, beta-tubulin (Giardia intestinalis, Giardiasis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein (Burkholderia mallei, Glanders); cyclophilin CyP, 24 kDa third-stage larvae protien GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR (Neisseria gonorrhoeae, Gonorrhea); outer membrane protein A OmpA, outer membrane protein C OmpC, outer membrane protein K17 OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emm1, M protein type 6 Emm6, immunoglobulin-binding protein 35 Sib35, Surface protein R28 Spr28, superoxide dismutase SOD, C5a peptidase ScpA, antigen I/II AgI/II, adhesin AspA, G-related alpha2-macroglobulin-binding protein GRAB, surface fibrillar protein M5 (Streptococcus pyogenes, Group A streptococcal infection); C protein P3 antigen, arginine deiminase proteins, adhesin BibA, 105 kDA protein BPS, surface antigens c, surface antigens R, surface antigens X, trypsin-resistant protein R1, trypsin-resistant protein R3, trypsin-resistant protein R4, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alpha-like protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein P1, outer membrane protein P2, outer membrane lipoprotein Pcp, Lipoprotein E, outer membrane protein P4, fuculokinase FucK, [Cu,Zn]-superoxide dismutase SodC, protease HtrA, protein 0145, alpha-galactosylceramide (Haemophilus influenzae, Haemophilus influenzae infection); polymerase 3D, viral capsid protein VP1, viral capsid protein VP2, viral capsid protein VP3, viral capsid protein VP4, protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein S, envelope glycoprotein G1, nucleoprotein NP, protein N, polyprotein M (Sin Nombre virus, Hantavirus, Hantavirus Pulmonary Syndrome (HPS)); heat shock protein HspA, heat shock protein HspB, citrate synthase GltA, protein UreB, heat shock protein Hsp60, neutrophil-activating protein NAP, catalase KatA, vacuolating cytotoxin VacA, urease alpha UreA, urease beta Ureb, protein Cpn10, protein groES, heat shock protein Hsp10, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, O-antigen, toxin-antigens Stx2B, toxin-antigen Stx1B, adhesion-antigen fragment Int28, protein EspA, protein EspB, Intimin, protein Tir, protein IntC300, protein Eae (Escherichia coli O157:H7, O111 and O104:H4, Hemolytic-uremic syndrome (HUS)); RNA polymerase L, protein L, glycoprotein Gn, glycoprotein Gc, nucleocapsid protein S, envelope glycoprotein G1, nucleoprotein NP, protein N, polyprotein M (Bunyaviridae family, Hemorrhagic fever with renal syndrome (HFRS)); glycoprotein G, matrix protein M, nucleoprotein N, fusion protein F, polymerase L, protein W, proteinC, phosphoprotein p, non-structural protein V (Henipavirus (Hendra virus Nipah virus), Henipavirus infections); polyprotein, glycoproten Gp2, hepatitis A surface antigen HBAg, protein 2A, virus protein VP1, virus protein VP2, virus protein VP3, virus protein VP4, protein P1B, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 (Hepatitis B Virus (HBV), Hepatitis B); envelope glycoprotein E1 gp32 gp35, envelope glycoprotein E2 NS1 gp68 gp70, capsid protein C, core protein Core, polyprotein, virus protein VP1, virus protein VP2, virus protein VP3, virus protein VP4, antigen G, protein NS3, protein NS5A, (Hepatitis C Virus, Hepatitis C); virus protein VP1, virus protein VP2, virus protein VP3, virus protein VP4, large hepaptitis delta antigen, small hepaptitis delta antigen (Hepatitis D Virus, Hepatitis D); virus protein VP1, virus protein VP2, virus protein VP3, virus protein VP4, capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase UL8, replication origin-binding protein UL9, glycoprotein M UL10, protein UL11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein IE63 (ICP27, UL54), protein UL55, protein UL56, viral replication protein ICP22 (IE68, US1), protein US2, serine/threonine-protein kinase US3, glycoprotein G (US4), glycoprotein J (US5), glycoprotein D (US6), glycoprotein I (US7), glycoprotein E (US8), tegument protein US9, capsid/tegument protein US10, Vmw21 protein (US11), ICP47 protein (IE12, US12), major transcriptional activator ICP4 (IE175, RS1), E3 ubiquitin ligase ICPO (IE110), latency-related protein 1 LRP1, latency-related protein 2 LRP2, neurovirulence factor RL1 (ICP34.5), latency-associated transcript LAT (Herpes simplex virus 1 and 2 (HSV-1 and HSV-2), Herpes simplex); heat shock protein Hsp60, cell surface protein H1C, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, glutathione S-transferase GST, aspartic protease APR-1, acetylcholinesterase AChE (Ancylostoma duodenale and Necator americanus, Hookworm infection); protein NS1, protein NP1, protein VP1, protein VP2, protein VP3 (Human bocavirus (HBOV), Human bocavirus infection); major surface protein 2 MSP2, major surface protein 4 MSP4, MSP variant SGV1, MSP variant SGV2, outer membrane protein OMP, outer membrane protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia ewingii, Human ewingii ehrlichiosis); major surface proteins 1-5 (MSP1a, MSP1b, MSP2, MSP3, MSP4, MSP5), type IV secreotion system proteins VirB2, VirB7, VirB11, VirD4 (Anaplasma phagocytophilum, Human granulocytic anaplasmosis (HGA)); protein NS1, small hydrophobic protein NS2, SH protein, fusion protein F, glycoprotein G, matrix protein M, matrix protein M2-1, matrix protein M2-2, phosphoprotein P, nucleoprotein N, polymerase L (Human metapneumovirus (hMPV), Human metapneumovirus infection); major surface protein 2 MSP2, major surface protein 4 MSP4, MSP variant SGV1, MSP variant SGV2, outer membrane protein OMP, outer membrane protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia chaffeensis, Human monocytic ehrlichiosis); replication protein E1, regulatory protein E2, protein E3, protein E4, protein E5, protein E6, protein E7, protein E8, major capsid protein L1, minor capsid protein L2 (Human papillomavirus (HPV), Human papillomavirus (HPV) infection); fusion protein F, hemagglutinin-neuramidase HN, glycoprotein G, matrix protein M, phosphoprotein P, nucleoprotein N, polymerase L (Human parainfluenza viruses (HPIV), Human parainfluenza virus infection); Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu)); genome polyprotein, protein E, protein M, capsid protein C (Japanese encephalitis virus, Japanese encephalitis); RTX toxin, type IV pili, major pilus subunit PilA, regulatory transcription factors PilS and PiIR, protein sigma54, outer membrane proteins (Kingella kingae, Kingella kingae infection); prion protein (Kuru prion, Kuru); nucleoprotein N, polymerase L, matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase F1, beta-tubulin, heat shock protein 70 Hsp70, KMP-11, glycoprotein GP63, protein BT1, nucleoside hydrolase NH, cell surface protein B1, ribosomal protein P1-like protein P1, sterol 24-c-methyltransferase SMT, LACK protein, histone H1, SPB1 protein, thiol specific antioxidant TSA, protein antigen STI1, signal peptidase SP, histone H2B, surface antigen PSA-2, cystein proteinase b Cpb (Leishmania genus, Leishmaniasis); major membrane protein I, serine-rich antigen-45 kDa, 10 kDa caperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycP1 or ML0041 coding protein, htrA2 or ML0176 coding protein, htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpL1, outer membrane protein LipL41, protein LigAni, surface protein LcpA, adhesion protein LipL53, outer membrane protein UpL32, surface protein Lsa63, flagellin FlaB1, membrane lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin O precursor Hly (LLO), invasion-associated protein lap (P60), Listeriolysin regulatory protein PrfA, Zinc metalloproteinase Mpl, Phosphatidylinositol-specific phospholipase C PLC (PICA, PlcB), O-acetyltransferase Oat, ABC-transporter permease Im.G_1771, adhesion protein LAP, LAP receptor Hsp60, adhesin LapB, haemolysin listeriolysin O LLO, protein ActA. Internalin A InIA, protein InIB (Listeria monocytogenes, Listeriosis); outer surface protein A OspA, outer surface protein OspB, outer surface protein OspC, decorin binding protein A DbpA, decorin binding protein B DbpB, flagellar filament 41 kDa core protein Fla, basic membrane protein A BmpA (Immunodominant antigen P39), outer surface 22 kDa lipoprotein precursor (antigen IPLA7), variable surface lipoprotein vIsE (usually Borrelia burgdorferi and other Borrelia species, Lyme disease (Lyme borreliosis)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, protein Cox-2 (Wuchereria bancrofti and Brugia malayi, Lymphatic filariasis (Elephantiasis)); glycoprotein GP, matrix protein Z, polymerase L, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMA1, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP8, enolase PF10, hepatocyte erythrocyte protein 17 kDa HEP17, erythrocyte membrane protein 1 EMP1, protein Kbetamerozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, polymerase L, matrix protein VP40, envelope glycoprotein GP (Marburg virus, Marburg hemorrhagic fever (MHF)); protein C, matrix protein M, phosphoprotein P, non-structural protein V, hemagglutinin glycoprotein H, polymerase L, nucleoprotein N, fusion protein F (Measles virus, Measles); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia pseudomallei, Melioidosis (Whitmore's disease)); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR, factor H-binding protein fHbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar ot integral membrane protein YIPA, anti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly (A) polymerase catalytic subunit PAPL, Major envelope protein P43K, early transcription factor 70 kDa subunit VETFS, early transcription factor 82 kDa subunit VETFL, metalloendopeptidase G1-type, nucleoside triphosphatase I NPH1, replication protein A28-like MC134L, RNA polymease 7 kDa subunit RPO7 (Molluscum contagiosum virus (MCV), Molluscum contagiosum (MC)); matrix protein M, phosphoprotein P/V, small hydrophobic protein SH, nucleoprotein N, protein V, fusion glycoprotein F, hemagglutinin-neuraminidase HN, RNA polymerase L (Mumps virus, Mumps): Outer membrane proteins OM, cell surface antigen OmpA, cell surface antigen OmpB (sca5), cell surface protein SCA4, cell surface protein SCA1, intracytoplasmic protein D, crystalline surface layer protein SLP, protective surface protein antigen SPA (Rickettsia typhi, Murine typhus (Endemic typhus)); adhesin P1, adhesion P30, protein p116, protein P40, cytoskeletal protein HMW1, cytoskeletal protein HMW2, cytoskeletal protein HMW3, MPN152 coding protein, MPN426 coding protein, MPN456 coding protein, MPN-500 coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Onchocerca volvulus, Onchocerciasis (River blindness)); 43 kDa secreted glycoprotein, glycoprotein gp0, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cystein protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, prtein PM0305, formate dehydrogenase-N, protein PM0698, protein PM1422, DNA gyrase, lipoprotein PIPE, adhesive protein Cp39, heme aquisition system receptor HasR, 39 kDa capsular protein, iron-regulated OMP IROMP, outer membrane protein OmpA87, fimbrial protein Ptf, fimbrial subunit protein PtfA, transferrin binding protein TbpI, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Prn, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Prn, protein Fim2, protein Fim3; “(Bordetella pertussis, Pertussis (Whooping cough)); “F1 capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YP00612, heat shock protein 90 HtpG, putative sulfatase protein YdeN, outer-membrane lipoprotein carrier protein LolA, secretion chaperone YerA, putative lipoprotein YP00420, hemolysin activator protein HpmB, pesticin/yersiniabactin outer membrane receptor Psn, secreted effector protein YopE, secreted effector protein YopF, secreted effector protein YopK, outer membrane protein YopN, outer membrane protein YopM, Coagulase/fibrinolysin precursor Pla; “(Yersinia pestis, Plague); protein PhpA, surface adhesin PsaA, pneumolysin Ply. ATP-dependent protease Clp, lipoate-protein ligase LplA, cell wall surface anchored protein psrP, sortase SrtA, glutamyl-tRNA synthetase GItX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A 12, 55 kDa antigen P55, major surface glycoprotein Msg (Pneumocystis jirovecii, Pneumocystis pneumonia (PCP)); genome polyprotein, polymerase 3D, viral capsid protein VP1, viral capsid protein VP2, viral capsid protein VP3, viral capsid protein VP4, protease 2A, protease 3C (Poliovirus, Poliomyelitis); protein Nfa1, exendin-3, secretory lipase, cathepsin B-like protease, cysteine protease, cathepsin, peroxiredoxin, protein Cry1Ac (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila psittaci, Psittacosis); outer membrane protein P1, heat shock protein B HspB, peptide ABC transporter, GTP-binding protein, protein IcmB, ribonuclease R, phosphatas SixA, protein DsbD, outer membrane protein ToIC, DNA-binding protein PhoB, ATPase DotB, heat shock protein B HspB, membrane protein Com1, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L, phophoprotein P, matrix protein M, glycoprotein G (Rabies virus, Rabies); fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 (Respiratory syncytial virus (RSV), Respiratory syncytial virus infection); genome polyprotein, polymerase 3D, viral capsid protein VP1, viral capsid protein VP2, viral capsid protein VP3, viral capsid protein VP4, protease 2A, protease 3C (Rhinovirus, Rhinovirus infection); outer membrane proteins OM, cell surface antigen OmpA, cell surface antigen OmpB (sca5), cell surface protein SCA4, cell surface protein SCA1, protein PS120, intracytoplasmic protein D, protective surface protein antigen SPA (Rickettsia genus, Rickettsial infection); outer membrane proteins OM, cell surface antigen OmpA, cell surface antigen OmpB (sca5), cell surface protein SCA4, cell surface protein SCA1, intracytoplasmic protein D (Rickettsia akari, Rickettsialpox); envelope glycoprotein GP, polymerase L, nucleoprotein N, non-structural protein NSS (Rift Valley fever virus, Rift Valley fever (RVF)); outer membrane proteins OM, cell surface antigen OmpA, cell surface antigen OmpB (sca5), cell surface protein SCA4, cell surface protein SCA1, intracytoplasmic protein D (Rickettsia rickettsii, Rocky mountain spotted fever (RMSF)); non-structural protein 6 NS6, non-structural protein 2 NS2, intermediate capsid protein VP6, inner capsid protein VP2, non-structural protein 3 NS3, RNA-directed RNA polymerase L, protein VP3, non-structural protein 1 NS1, non-structural protein 5 NS5, outer capsid glycoprotein VP7, non-structural glycoprotein 4 NS4, outer capsid protein VP4; (Rotavirus, Rotavirus infection); polyprotein P200, glycoprotein E1, glycoprotein E2, protein NS2, capsid protein C (Rubella virus, Rubella); chaperonin GroEL (MopA), inositol phosphate phosphatase SopB, heat shock protein HsIU, chaperone protein DnaJ, protein TviB, protein IroN, flagellin FliC, invasion protein SipC, glycoprotein gp43, outer membrane protein LamB, outer membrane protein PagC, outer membrane protein TolC, outer membrane protein NmpC, outer membrane protein FadL, transport protein SadA, transferase WgaP, effector proteins SifA, SteC, SseL, SseJ and SseF (Salmonella genus, Salmonellosis); “protein 14, non-structural protein NS7b, non-structural protein NS8a, protein 9b, protein 3a, nucleoprotein N, non-structural protein NS3b, non-structural protein NS6, protein 7a, non-structural protein NS8b, membrane protein M, envelope small membrane protein EsM, replicase polyprotein 1a, spike glycoprotein S, replicase polyprotein lab; SARS coronavirus, SARS (Severe Acute Respiratory Syndrome)); serin protease, Atypical Sarcoptes Antigen 1 ASA1, glutathione S-transferases GST, cystein protease, serine protease, apolipoprotein (Sarcoptes scabiei, Scabies); glutathione S-transferases GST, paramyosin, hemoglbinase SM32, major egg antigen, 14 kDa fatty acid-binding protein Sm14, major larval surface antigen P37, 22.6 kDa tegumental antigen, calpain CANP, triphospate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator IE63, transcriptional transactivator IE62, protease P33, alpha trans-inducing factor 74 kDa protein, deoxyuridine 5′-triphosphate nucleotidohydrolase, transcriptional transactivator IE4, membrane protein UL43 homolog, nuclear phosphoprotein UL3 homolog, nuclear protein UL4 homolog, replication origin-binding protein, membrane protein 2, phosphoprotein 32, protein 57,DNA polymerase processivity factor, portal protein 54, DNA primase, tegument protein UL14 homolog, tegument protein UL21 homolog, tegument protein UL55 homolog, tripartite terminase subunit UL33 homolog, tripartite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein C1, protein C5, protein C6, protein F7, protein F8, protein F9, protein F11, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A CIfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A CIfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, e.g., aureus, Staphylococcal infection); antigen Ss-IR, antigen NIE, strongylastacin, Na+-K+ ATPase Sseat-6, tropomysin SsTmy-1, protein LEC-5, 41 kDa aantigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpF1, repeat protein Tpr, repeat protein F TprF, repeat protein G TprG, repeat protein | Tprl, repeat protein J TprJ, repeat protein K TprK, treponemal membrane protein A TmpA, lipoprotein, 15 kDa Tpp15, 47 kDa membrane antigen, miniferritin TpF1, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); genome polyprotein, protein E, protein M, capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES-70, glycoprotein GP31, excretory-secretory antigen TcES-57, perienteric fluid antigen Pe, soluble extract antigens Ex, excretory/secretory larval antigens ES, antigen TES-120, polyprotein allergen TBA-1, cathepsin L-like cysteine protease c-cpl-1, 26-kDa protein (Toxocara canis or Toxocara cati, Toxocariasis (Ocular Larva Migrans (OLM) and Visceral Larva Migrans (VLM))); microneme proteins (MIC1, MIC2, MIC3, MIC4, MIC5, MIC6, MIC7, MIC8), rhoptry protein Rop2, rhoptry proteins (Rop1, Rop2, Rop3, Rop4, Rop5, Rop6, Rop7, Rop16, Rjop17), protein SRi, surface antigen P22, major antigen p24, major surface antigen p30, dense granule proteins (GRA1, GRA2, GRA3, GRA4, GRA5, GRA6, GRA7, GRA8, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Myb1, Myb2, Myb3), adhesion protein AP23, adhesion protein AP33, adhesin protein AP33-3, adhesins AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Enol, cysteine proteinase CP30, heat shock proteins (Hsp70, Hsp60), immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone Dnak, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTB8, Rpf-like proteins, protein MTB32, protein MTB39, crystallin, heat-shock protein HSP65, protein PST-S(usually Mycobacterium tuberculosis, Tuberculosis); outer membrane protein FobA, outer membrane protein FobB, intracellular growth locus IgIC1, intracellular growth locus IgIC2, aminotransferase Wbt1, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein tolC, FAD binding family protein, type IV pilin multimeric outer membrane protein, two component sensor protein KdpD, chaperone protein DnaK, protein TolQ (Francisella tularensis, Tularemia); “MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen; “(Ureaplasma urealyticum, Ureaplasma urealyticum infection); non-structural polyprotein, structural polyprotein, capsid protein CP, protein E1, protein E2, protein E3, protease P1, protease P2, protease P3 (Venezuelan equine encephalitis virus, Venezuelan equine encephalitis); glycoprotein GP, matrix protein Z, polymerase L, nucleoprotein N (Guanarito virus, Venezuelan hemorrhagic fever); polyprotein, protein E, protein M, capsid protein C, protease NS3, protein NS1, protein NS2A, protein AS2B, brotein NS4A, protein NS4B, protein NS5 (West Nile virus, West Nile Fever); cpasid protein CP, protein E1, protein E2, protein E3, protease P2 (Western equine encephalitis virus, Western equine encephalitis); genome polyprotein, protein E, protein M, capsid protein C, protease NS3, protein NS1, protein NS2A, protein AS2B, protein NS4A, protein NS4B, protein NS5 (Yellow fever virus, Yellow fever); putative Yop targeting protein YobB, effector protein YopD, effector protein YopE, protein YopH, effector protein YopJ, protein translocation protein YopK, effector protein YopT, protein YpkA, flagellar biosyntheses protein FlhA, peptidase M48, potassium efflux system KefA, transcriptional regulatoer RovA, adhesin Ifp, translocator portein LcrV, protein PcrV, invasin Inv, outer membrane protein OmpF-like porin, adhesin YadA, protein kinase C, phospholipase C1, protein PsaA, mannosyltransferase-like protein WbyK, protein YscU, antigen YPMa (Yersinia pseudotuberculosis, Yersinia pseudotuberculosis infection); effector protein YopB, 60 kDa chaperonin, protein WbcP, tyrosin-protein phosphatase YopH, protein YopQ, enterotoxin, Galactoside permease, reductaase NrdE, protein YasN, Invasin Inv, adhesin YadA, outer membrane porin F OmpF, protein UspA1, protein EibA, protein Hia, cell surface protein Ail, chaperone SycD, protein LcrD, protein LcrG, protein LcrV, protein SycE, protein YopE, regulator protein TyeA, protein YopM, protein YopN, protein YopO, protein YopT, protein YopD, protease ClpP, protein MyfA, protein FilA, and protein PsaA (Yersinia enterocolitica, Yersiniosis). The brackets in this paragraph indicate the particular pathogen or the family of pathogens of which the antigen(s) is/are derived and the infectious disease with which the pathogen is associated.

In certain embodiments, the protein of interest can be a tumor antigen, or a fragment or variant thereof, wherein the tumor antigen is preferably selected from the group consisting of 1A01_HLA-A/m; 1A02; 5T4; ACRBP; AFP; AKAP4; alpha-actinin-_4/m; alpha-methylacyl-coenzyme_A_racemase; ANDR; ART-4; ARTC1/m; AURKB; B2MG; B3GN5; B4GN1; B7H4; BAGE-1; BASI; BCL-2; bcr/abl; beta-catenin/m; BING-4; BIRC7; BRCA1/m; BY55; calreticulin; CAMEL; CASP-8/m; CASPA; cathepsin_B; cathepsin_L; CD1A; CD1B; CD1C; CD1D; CD1E; CD20; CD22; CD276; CD33; CD3E; CD3Z; CD44_Isoform_1; CD44_Isoform_6; CD4; CD52; CD55; CD56; CD80; CD86; CD8A; CDC27/m; CDE30; CDK4/m; CDKN2A/m; CEA; CEAM6; CH3L2; CLCA2; CML28; CML66; COA-1/m; coactosin-like_protein; collagen_XXIII; COX-2; CP1B1; CSAG2; CT45A1; CT55; CT-_9/BRD6; CTAG2_Isoform_LAGE-1A; CTAG2_Isoform_LAGE-1B; CTCFL; Cten; cyclin_B1; cyclin_D1; cyp-B; DAM-10; DEP1A; E7; EF1A2; EFTUD2/m; EGFR; EGLN3; ELF2/m; EMMPRIN; EpCam; EphA2; EphA3; ErbB3; ERBB4; ERG; ETV6; EWS; EZH2; FABP7; FCGR3A_Version_1; FCGR3A_Version_2; FGF5; FGFR2; fibronectin; FOS; FOXP3; FUT1; G250; GAGE-1; GAGE-2; GAGE-3; GAGE-4; GAGE-5; GAGE-6; GAGE7b; GAGE-8_(GAGE-2D); GASR; GnT-V; GPC3; GPNMB/m; GRM3; HAGE; hepsin; Her2/neu; HLA-A2/m; homeobox_NKX3.1; HOM-TES-85; HPG1; HS71A; HS71B; HST-2; hTERT; ICE; IF2B3; IL10; IL-13Ra2; IL2-RA; IL2-RB; IL2-RG; IL-5; IMP3; ITA5; ITB1; ITB6; kallikrein-2; kallikrein-4; KI20A; KIAA0205; KIF2C; KK-LC-1; LDLR; LGMN; LIRB2; LY6K; MAGA5; MAGA8; MAGAB; MAGE-A10; MAGE-A12; MAGE-A1; MAGE-A2; MAGE-A3; MAGE-A4; MAGE-A6; MAGE-A9; MAGE-B10; MAGE-B16; MAGE-B17; MAGE-_B1; MAGE-B2; MAGE-B3; MAGE-B4; MAGE-B5; MAGE-B6; MAGE-C1; MAGE-C2; MAGE-C3; MAGE-D1; MAGE-D2; MAGE-D4; MAGE-_E1; MAGE-E1_(MAGE1); MAGE-E2; MAGE-F1; MAGE-H1; MAGEL2; mammaglobin_A; MART-1/melan-A; MART-2; MC1_R; M-CSF; mesothelin; MITF; MMP1_1; MMP7; MUC-1; MUM-1/m; MUM-2/m; MYCN; MYO1A: MYO1B; MYO1C; MYO1D; MYO1E; MYO1F; MYO1G; MYO1H; NA17; NA88-A; Neo-PAP; NFYC/m; NGEP; NPM; NRCAM; NSE; NUF2; NY-ESO-1; OA1; OGT; OS-9; osteocalcin; osteopontin; p53; PAGE-4; PAI-1; PAI-2; PAP; PATE; PAX3; PAX5; PD1L1; PDCD1; PDEF; PECA1; PGCB; PGFRB; Pim-1_-Kinase; Pin-1; PLAC1; PMEL; PML; POTEF; POTE; PRAME; PRDX5/m; PRM2; prostein; proteinase-3; PSA; PSB9; PSCA; PSGR; PSM; PTPRC; RAB8A; RAGE-1; RARA; RASH; RASK; RASN; RGS5; RHAMM/CD168; RHOC; RSSA; RU1; RU2; RUNX1; S-100; SAGE; SART-_1; SART-2; SART-3; SEPR; SERPINB5; SIA7F; SIA8A; SIAT9; SIRT2/m; SOX10; SP17; SPNXA; SPXN3; SSX-1; SSX-2; SSX3; SSX-4; ST1A1; STAG2; STAMP-1; STEAP-1; Survivin-2B; survivin; SYCP1; SYT-SSX-1; SYT-SSX-2; TARP; TCRg; TF2AA; TGFB1; TGFR2; TGM-4; TIE2; TKTL1; TPI/m; TRGV11; TRGV9; TRPC1; TRP-p8; TSG10; TSPY1; TVC_(TRGV3); TX101; tyrosinase; TYRP1; TYRP2; UPA; VEGFR1; WT1; and XAGE1.

In certain embodiments, the proteins of interest differ and can contain substantial regions of similar immunological epitopes recognized similarly by the immune system (i.e., a conserved element). A “conserved element” as used herein refers to a protein sequence that is conserved across a protein that has high sequence diversity in nature, e.g., a viral protein such as a gag. The conserved element need not have 100% sequence identity across the diversity of naturally occurring sequence of the protein, but the sequence variability in the naturally occurring sequences is low, e.g., less than 20%. In some embodiments, the sequence variability is less than 10%. A conserved element is usually eight amino acids, or greater, e.g., 9, 10, 11, 12, 13, 14, 15, 16, 17, 18, 19, or 20 amino acids in length. Typically a conserved element is less than 50 amino acids in length and often is less than 40 or less than 30 amino acids. In some embodiments, a conserved element is less than 25 amino acids in length.

Conserved elements of a protein of interest can be determined using known methods. For examples U.S. Patent Application Publication No. 20110269937, which is incorporated by reference, describes methods of evaluating protein sequences that exhibit natural variability to identify regions that are conserved using computational methods.

A conserved element nucleic acid construct is typically generated by linking nucleic acid sequences that encode multiple conserved elements that target conserved sequence that are present within all or a high percentage, e.g., at least 80%, at least 90%, or at least 95%, or greater, of the naturally occurring variants of the protein in a population. In typical embodiments, a conserved element is from a region of a protein that when mutated, has deleterious effects on the function of the protein. In typical embodiments, a conserved element does not comprise an amino acid sequence that does not occur in a naturally occurring variant, i.e., the conserved element does not contain amino acid substitutions that would result in a sequence that has not been identified in a naturally occurring variant.

The terms “identical” or percent “identity,” in the context of two or more nucleic acids or polypeptide sequences, refer to two or more sequences or subsequences that are the same or have a specified percentage of amino acid residues or nucleotides that are the same (e.g., about 80%, 85%, 90%, 91%, 92%, 93%, 94%, 95%, 96%, 97%, 98%, 99%, or higher identity over a specified region (a polypeptide sequence comprising conserved elements), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A preferred example of algorithm that is suitable for determining percent sequence identity and sequence similarity are the BLAST algorithms, which are described in Altschul et al., Nuc. Acids Res. 25:3389-3402 (1977) and Altschul et al., J. Mol. Biol. 215:403-410 (1990), respectively. BLAST software is publicly available through the National Center for Biotechnology Information on the worldwide web at ncbi.nlm.nih.gov/. Both default parameters or other non-default parameters can be used. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Conservatively modified variants” as used herein applies to amino acid sequences. One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see e.g., Creighton, Proteins (1984)).

### Immune Response

The methods provided herewith induce optimal, effective, and balanced humoral and cellular immunity. As used herein, an “immune response” can refer to either a specific reaction of the adaptive immune system to a particular protein of interest or antigen (so called specific or adaptive immune response) or an unspecific reaction of the innate immune system (so called unspecific or innate immune response). In some embodiments, this disclosure relates to the specific reactions (adaptive immune responses) of the adaptive immune system. Particularly, it relates to adaptive immune responses to infections by viruses, for example. However, this specific response can be supported by an additional unspecific reaction (innate immune response). Therefore, in certain embodiments, this disclosure also relates to methods for stimulation of the innate and the adaptive immune system to evoke an efficient adaptive immune response.

Adaptive immune system: The adaptive immune system is composed of highly specialized, systemic cells and processes that eliminate or prevent pathogenic growth. The adaptive immune response provides the vertebrate immune system with the ability to recognize and remember specific pathogens (to generate immunity), and to mount stronger attacks each time the pathogen is encountered. The system is highly adaptable because of somatic hypermutation (a process of increased frequency of somatic mutations), and V (D) J recombination (an irreversible genetic recombination of antigen receptor gene segments). This mechanism allows a small number of genes to generate a vast number of different antigen receptors, which are then uniquely expressed on each individual lymphocyte. Because the gene rearrangement leads to an irreversible change in the DNA of each cell, all of the progeny (offspring) of that cell will then inherit genes encoding the same receptor specificity, including the Memory B cells and Memory T cells that are the keys to long-lived specific immunity. Immune network theory is a theory of how the adaptive immune system works, that is based on interactions between the variable regions of the receptors of T cells, B cells and of molecules made by T cells and B cells that have variable regions.

Adaptive immune response: The adaptive immune response is typically understood to be antigen-specific. Antigen specificity allows for the generation of responses that are tailored to specific antigens, pathogens or pathogen-infected cells. The ability to mount these tailored responses is maintained in the body by “memory cells”. Should a pathogen infect the body more than once, these specific memory cells are used to quickly eliminate it. In this context, the first step of an adaptive immune response is the activation of naive antigen-specific T cells or different immune cells able to induce an antigen-specific immune response by antigen-presenting cells. This occurs in the lymphoid tissues and organs through which naive T cells are constantly passing. Cell types that can serve as antigen-presenting cells are inter alia dendritic cells, macrophages, and B cells. Each of these cells has a distinct function in eliciting immune responses. Dendritic cells take up antigens by phagocytosis and macropinocytosis and are stimulated by contact with, e.g., a foreign antigen to migrate to the local lymphoid tissue, where they differentiate into mature dendritic cells. Macrophages ingest particulate antigens such as bacteria and are induced by infectious agents or other appropriate stimuli to express MHC molecules. The unique ability of B cells to bind and internalize soluble protein antigens via their receptors may also be important to induce T cells. Presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which do not recognize and bind antigen directly, but instead recognize short peptide fragments, e.g., of pathogen-derived protein antigens, which are bound to MHC molecules on the surfaces of other cells.

Cellular immunity/cellular immune response: Cellular immunity relates typically to the activation of macrophages, natural killer cells (NK), antigen-specific cytotoxic T-lymphocytes, and the release of various cytokines in response to an antigen. In a more general way, cellular immunity is not related to antibodies but to the activation of cells of the immune system. A cellular immune response is characterized, e.g., by activating antigen-specific cytotoxic T-lymphocytes that are able to induce apoptosis in body cells displaying epitopes of an antigen on their surface, such as virus-infected cells, cells with intracellular bacteria, and cancer cells displaying tumor antigens; activating macrophages and natural killer cells, enabling them to destroy pathogens; and stimulating cells to secrete a variety of cytokines that influence the function of other cells involved in adaptive immune responses and innate immune responses.

Humoral immunity/humoral immune response: Humoral immunity refers typically to antibody production and the accessory processes that may accompany it. A humoral immune response may be typically characterized, e.g., by Th2 activation and cytokine production, germinal center formation and isotype switching, affinity maturation and memory cell generation. Humoral immunity also typically may refer to the effector functions of antibodies, which include pathogen and toxin neutralization, classical complement activation, and opsonin promotion of phagocytosis and pathogen elimination.

Innate immune system: The innate immune system, also known as non-specific immune system, comprises the cells and mechanisms that defend the host from infection by other organisms in a non-specific manner. This means that the cells of the innate system recognize and respond to pathogens in a generic way, but unlike the adaptive immune system, it does not confer long-lasting or protective immunity to the host. The innate immune system may be e.g., activated by ligands of pathogen-associated molecular patterns (PAMP) receptors, e.g., Toll-like receptors (TLRs) or other auxiliary substances such as lipopolysaccharides, TNF-alpha, CD40 ligand, or cytokines, monokines, lymphokines, interleukins or chemokines, IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, growth factors, and hGH, a ligand of human Toll-like receptor TLR1, TLR2, TLR3, TLR4, TLR5, TLR6, TLR7, TLR8, TLR9, TLR10, a ligand of murine Toll-like receptor TLR1, TLR2, TLR3, TLR4, TLR5, TLR6, TLR7, TLR8, TLR9, TLR 10, TLR11, TLR 12 or TLR13, a ligand of a NOD-like receptor, a ligand of a RIG-I like receptor, an immunostimulatory nucleic acid, an immunostimulatory RNA (isRNA), a CpG-DNA, an antibacterial agent, or an anti-viral agent. Typically a response of the innate immune system includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system through a process known as antigen presentation; and/or acting as a physical and chemical barrier to infectious agents.

The terms “enhanced immune response” or “increased immune response” as used herein refers to an immune response to the protein(s) of interest that are administered by the one or more priming doses and the one or more boosting doses, where the immune response is increased in comparison to when only the one or more priming doses is administered. An “enhanced immune response” may include increases in the level of immune cell activation and/or an increase in the duration of the response and/or immunological memory as well as an improvement in the kinetics of the immune response. The increase can be demonstrated by either a numerical increase, e.g., an increased in levels of antibody in a particular time frame, as assessed in an assay to measure the response assay or by prolonged longevity of the response.

Without limiting the disclosure, a number of embodiments of the disclosure are described below for purpose of illustration.

The subject matter will be further described in the following examples, which do not limit the scope of the subject matter described in the claims.

### EXAMPLES

The Examples that follow are illustrative of specific embodiments of the disclosure, and various uses thereof. They are set forth for explanatory purposes only, and should not be construed as limiting the scope of the claimed subject matter in any way.

### Materials and Methods

Animals and vaccines: Macaque vaccine studies were conducted in compliance with all the state and federal regulations and were approved by BIOQUAL's Institutional Animal Care and Use Committee (IACUC). The LNP-formulated RNActive vaccines encoding for HIV CE (p24CE) (16, 29) or gag (p55gag) (16, 29) are produced by CureVac AG, Tübingen, Germany, as detailed in (64). Lipid nanoparticle (LNP)-encapsulation of mRNA was performed by Acuitas Therapeutics (Vancouver, Canada). Fifteen naïve Indian rhesus macaques were enrolled in mRNA/LNP vaccination studies using 25 μg of mRNA in each vaccination. The animals were divided in three vaccine groups (n=5) with equal distribution of age, weight, and gender. All animals received 4 mRNA/LNP vaccinations by intramuscular injection onto the inner thigh at 0, 4, 12 and 24 weeks. Animals in group 1 were immunized with the CE mRNA/LNP vaccine only. Animals in group 2 received the gag mRNA/LNP only. Animals in group 3 were immunized twice with the CE mRNA/LNP vaccine followed by two vaccinations using a combination of the CE and gag mRNA/LNP vaccine. In a second study, five naïve animals were immunized twice (four weeks apart) with a 100 μg/dose of gag mRNA/LNP.

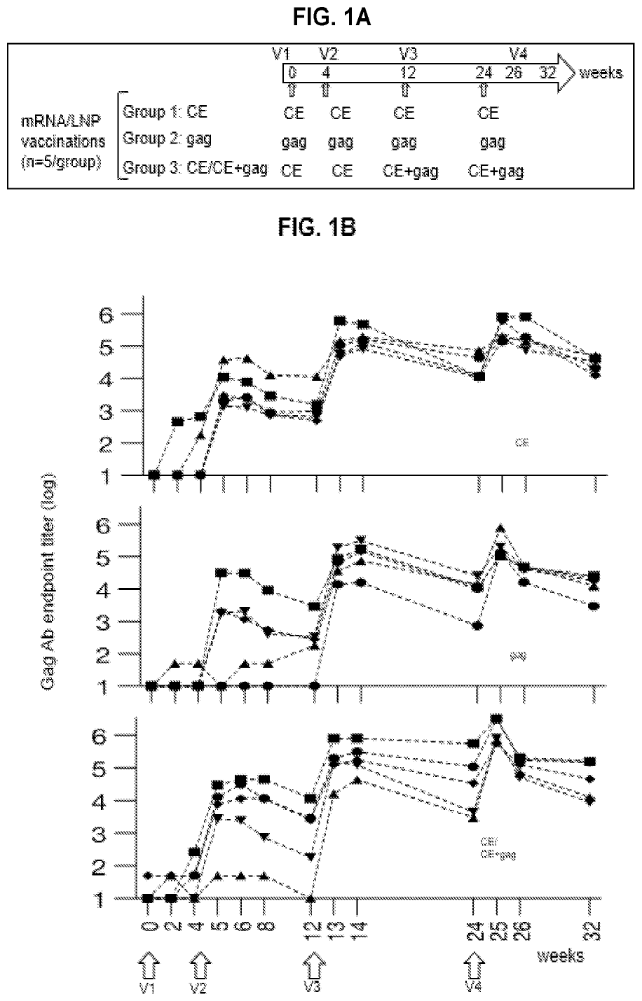

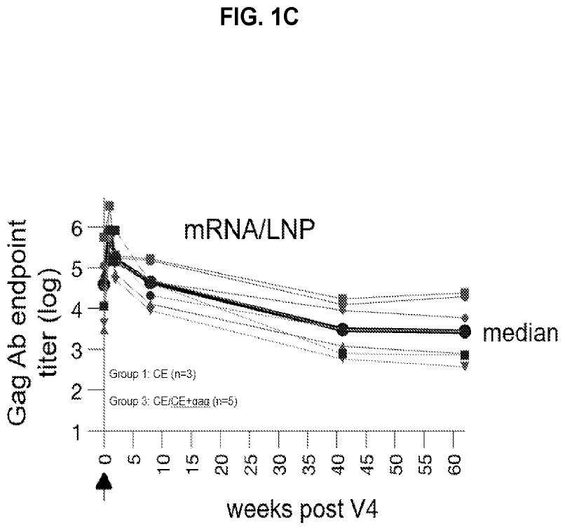

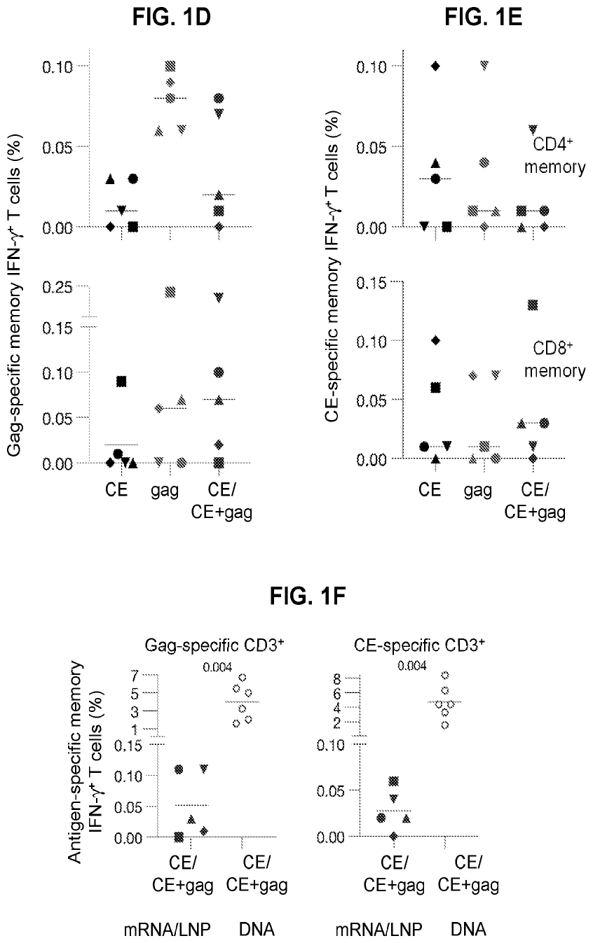

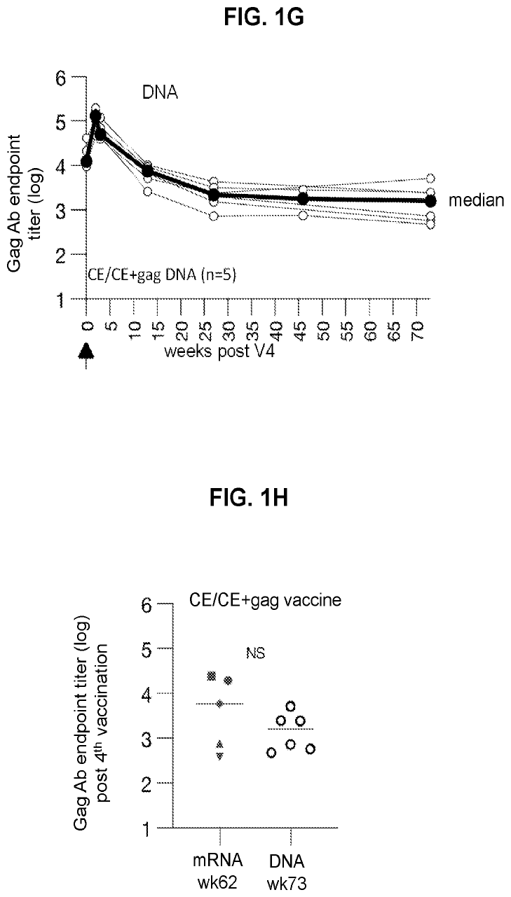

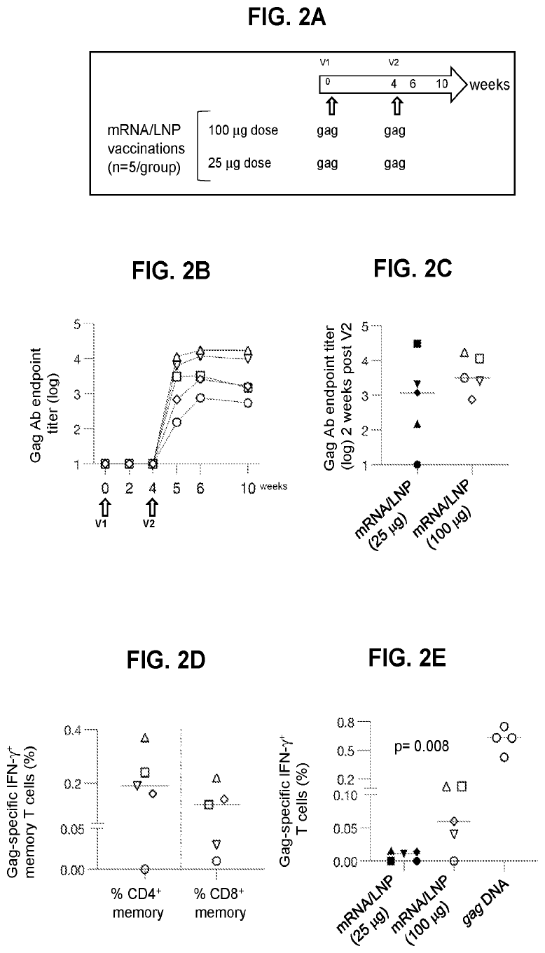

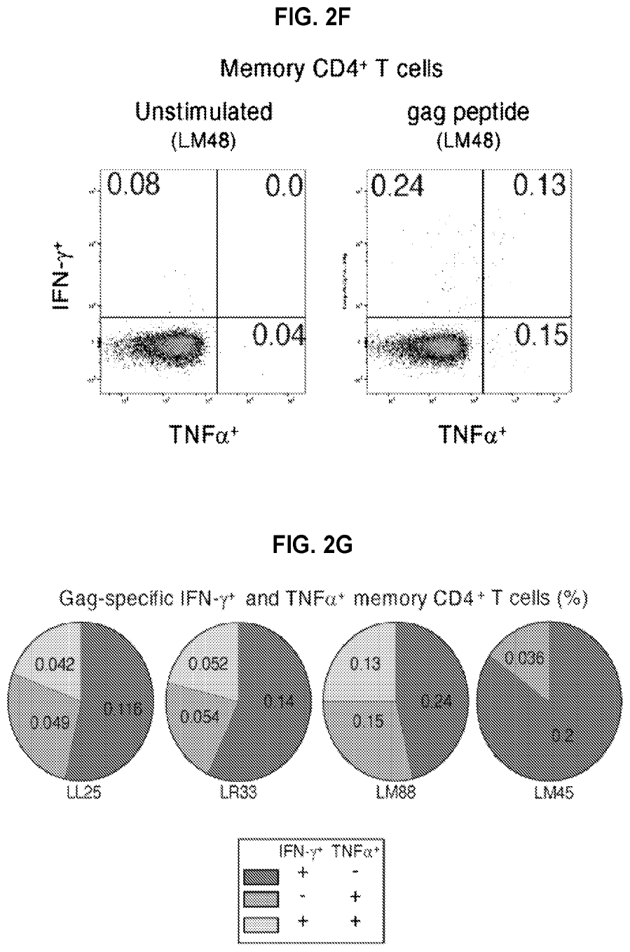

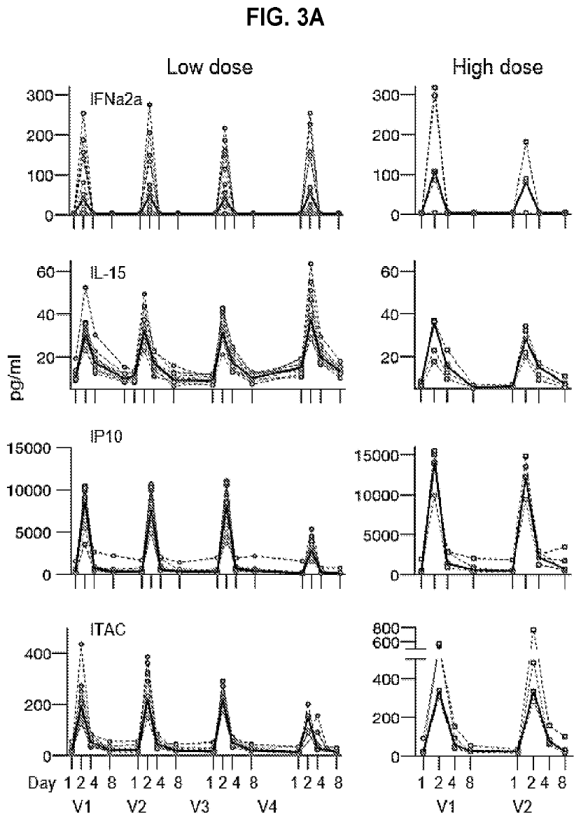

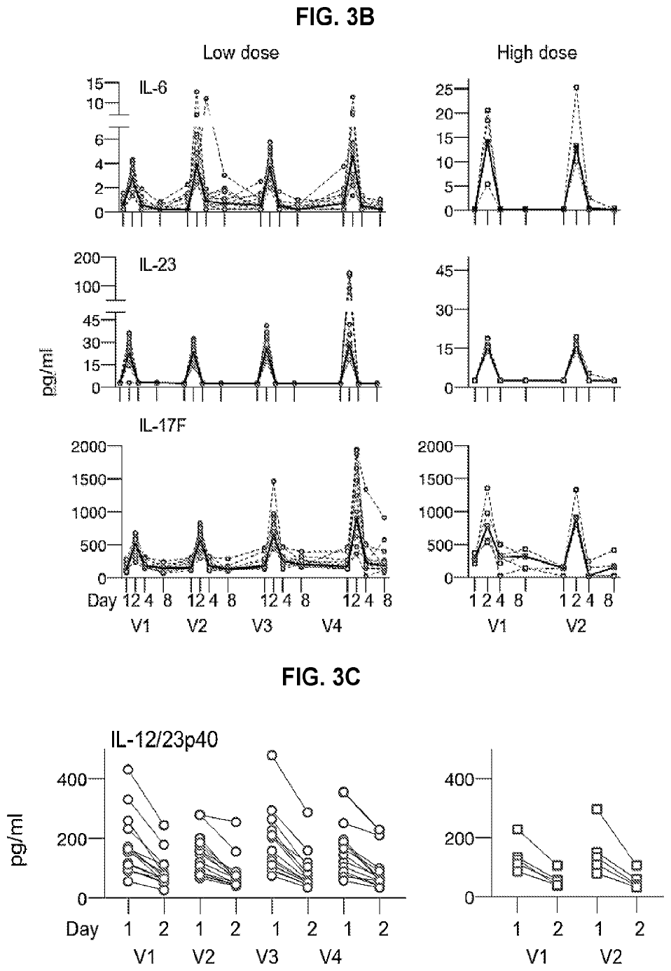

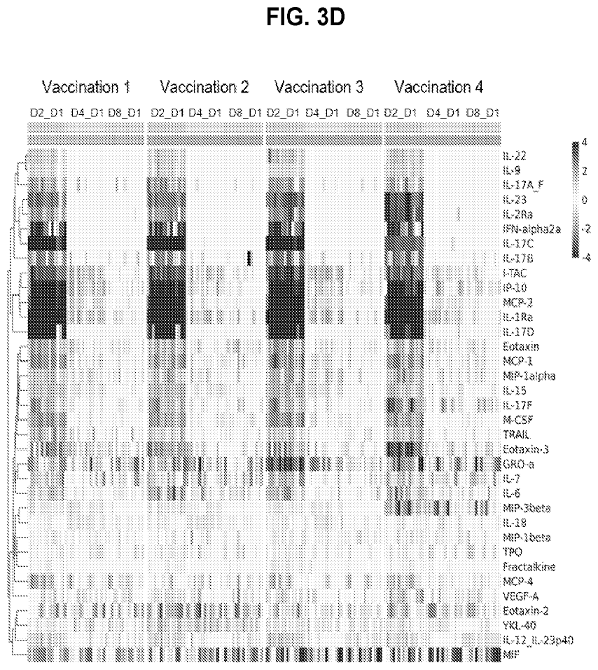

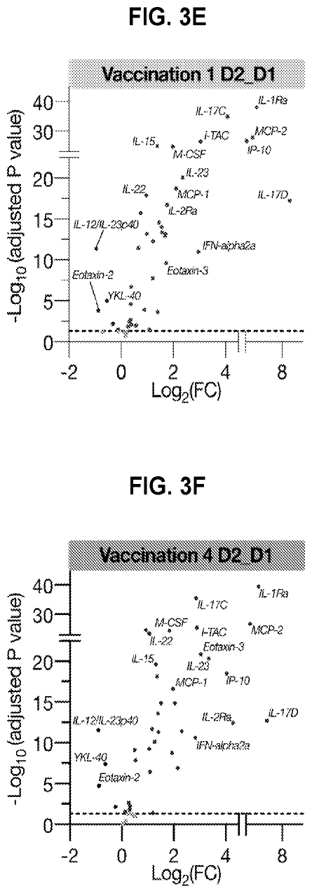

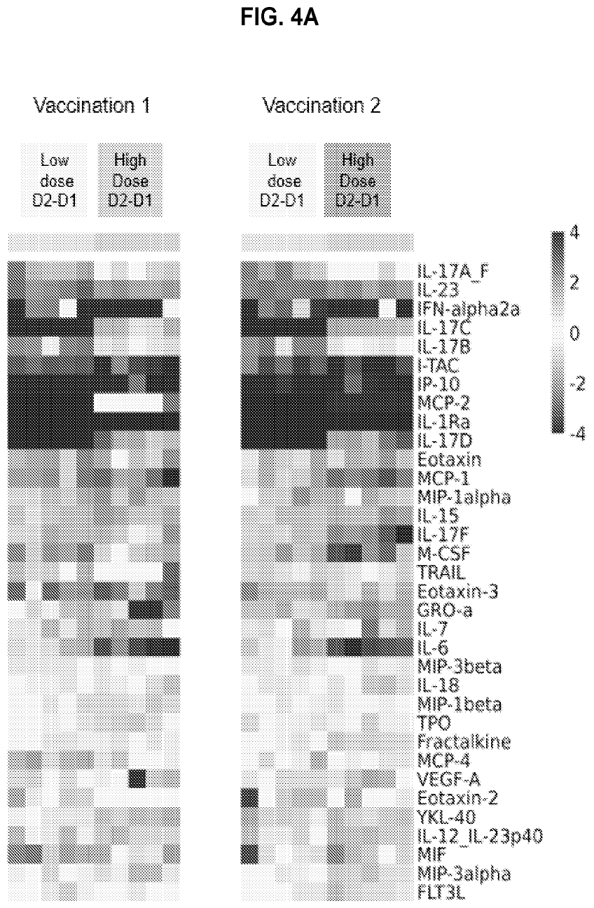

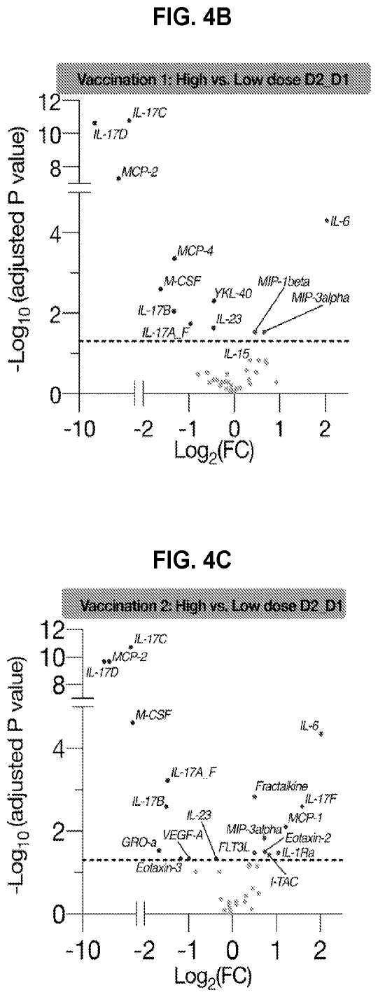

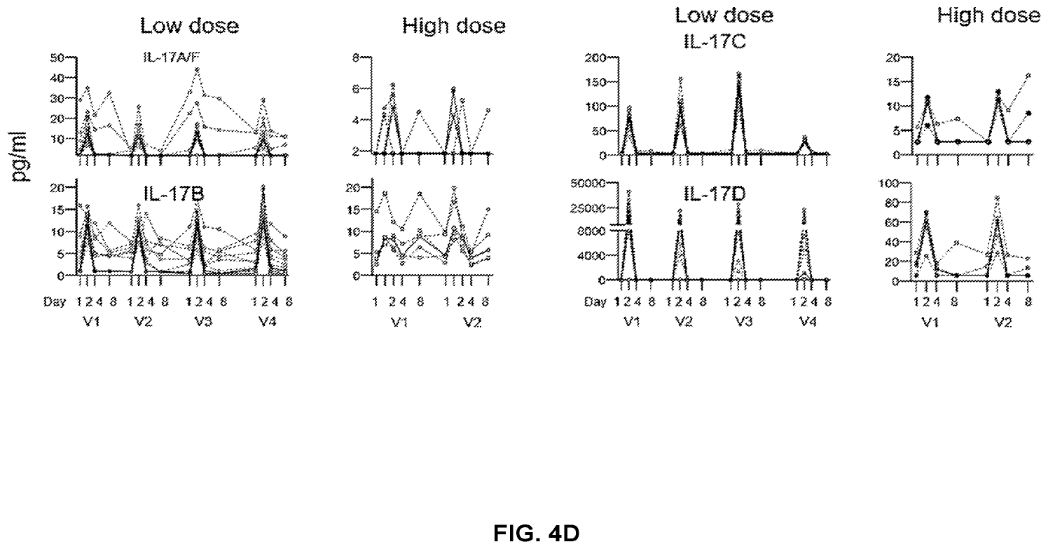

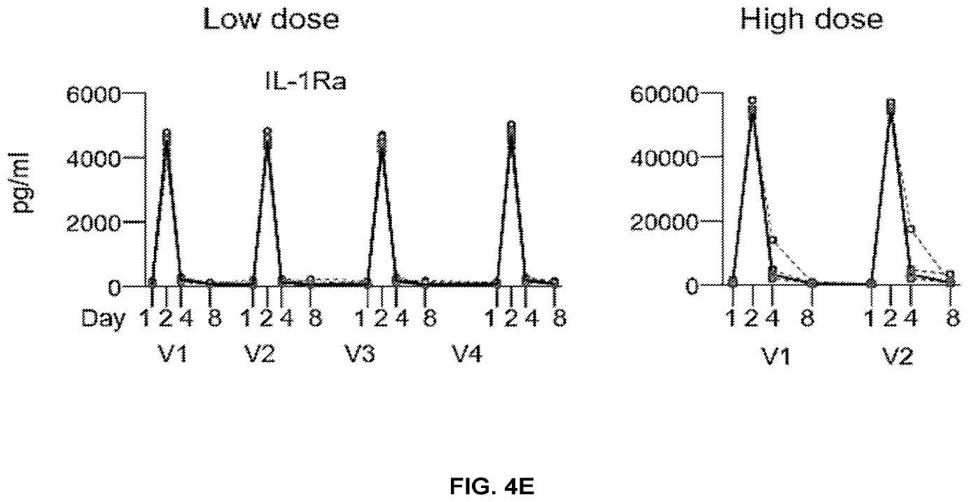

Booster vaccine studies of animals previously immunized with plasmid DNA were performed with 25 μg dose of the gag mRNA/LNP vaccine. Priming or booster vaccinations with gag DNA were performed by intramuscular injection followed by in vivo electroporation using the Cellectra 5P device (Inovio Pharmaceuticals, Inc). The DNAs p55^{gag} (plasmid 114H) and p24CE (plasmid 306H) expressed Gag and CE, respectively, from codon-optimized sequences inserted between the human cytomegalovirus (CMV) promoter and the bovine growth hormone (BGH) polyadenylation signal (29, 30). The DNA dose was: 4 mg (FIG. 1F-G), reported in (30); 1 mg (FIG. 2), reported in (29); and 2 mg (FIGS. 3 and 4). The vaccines also contained 0.2 mg IL-12 DNA, except for the 5 DNA primed macaques shown in FIGS. 3 and 4.

Cellular immune responses: Antigen-specific T cell responses were measured in peptide-stimulated PBMC in the presence of the protein secretion inhibitor Monensin (GolgiStop), as previously described (30). Briefly, 10⁶ cells were seeded in 96-well plates and stimulated with different peptide pools at a final concentration of 1 μg/ml for each individual peptide. For negative and positive controls, PBMC were cultured in medium without peptides or stimulated with a PMA-cell stimulation cocktail (eBioscience, Affymetrix Inc. San Diego, CA, USA). After 12 hours incubation, the cells were washed and stained with antibody cocktails targeting surface proteins. After 20 minutes of incubation, the cells were washed and fixed/permeabilized at 4° C. in the dark using the FoxP3 fixation/permeabilization buffer (eBioscience, Affymetrix Inc. San Diego, CA). After washing the cells with permeabilization buffer (eBioscience by Affymetrix Inc.), the cells were stained with an antibody mix targeting cytokines and intracellular proteins. After 30 minutes of incubation at room temperature, the samples were washed, resuspended in PBS and acquired on a Fortessa or BDSymphony flow cytometer (BD Biosciences, San Jose, CA). The flow data were analyzed using FlowJo software (BD Biosciences, San Jose, CA). The following antibodies (clones in parenthesis) were used in these studies: from BD Biosciences; CD3 (SP34-2), CD4 (L200), CD95 (DX2), CD69 (FN50), IFNγ (B27), Ki67 (B56), IL-2, (MQ1-17H12), TNFα (Mab11), granzyme B (GB11), CCR7 (3D12), MHC-II (TU39), CD16 (3G8); from Biolegend; CD8(RPA-T8), CD28 (CD28-2), PD-1 (EH12.2H7), CD137 (4B4-1), CXCR3 (G025H7); from ThermoFisher/eBioscience; CD107a (eBioH4A3), T-bet (4B10), Eomes (WD1928); from Mabtech; Perforin (Pf-344).

Humoral immune responses: Anti-p24Gag antibodies were measured by ELISA using eight 4-fold serial dilutions of plasma samples, starting at 1:50 dilution. The OD450 measurements of the diluted samples were plotted, and GraphPad Prism area-under-the-curve was used to determine the endpoint titers above the baseline using the last X feature. Linear endpoint titers were used for comparative analysis.

Cytokine measurements: Plasma samples, collected at the day of vaccination (day 1), and at day 2, 4, and 8 after each mRNA/LNP vaccination, were monitored using a U-PLEX Non-Human Primate Biomarker Assay (Meso Scale Diagnostics, MD, USA) for changes in the concentration of 61 cytokines/chemokines according to the manufacturer's instructions.

Bioinformatics and statistical analysis: The biomarker analysis was performed with a workflow written in R and through a user interface developed on the Foundry Platform (Palantir Technologies). Briefly, biomarkers falling below the detection limit/standard range were removed if absent in more than 50% of the samples or adjusted to 0.5 detection limit/standard point. The limma R package (v3.38.3) was used to compare biomarker changes between time points and R (v3.5.1) as implemented on the NIH Integrated Data Analysis Platform. Analysis was performed by using GraphPad Prism Version 9.2 for MacOS X (GraphPad Software, Inc, La Jolla, CA).

### Example 1: HIV CE and Gag mRNA/LNP Vaccination in Macaques

A cohort of 15 naïve rhesus macaques (5 per group) was vaccinated with 25 μg doses of HIV mRNA/lipid nanoparticle (LNP). Group 1 was vaccinated with HIV mRNAs expressing conserved elements in p24Gag (CE), a bivalent immunogen comprising of CE1 and CE2 differing by 7 amino acids to cover >98% of group M Gag; group 2 was vaccinated with mRNA expressing the complete p55Gag (gag), while group 3 was vaccinated with mRNA expressing a combination of CE prime followed by CE+Gag (12.5 μg each) boost (CE+gag) (FIG. 1A). The same protocol was previously tested in macaques using the DNA platform (30) and is currently being tested in clinical trials (NCT03181789; NCT03560258; NCT04357821).

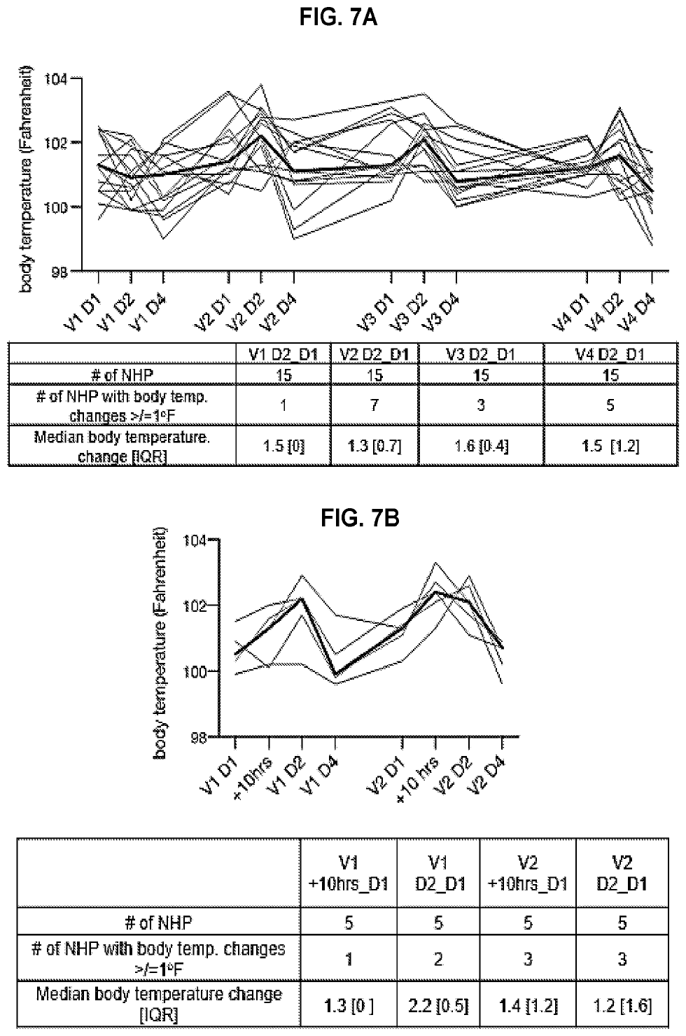

Vaccinations with the mRNA/LNP formulations were safe in rhesus macaques. Some animals had mildly elevated body temperature (>1° F.) 24 hours after vaccine delivery (FIG. 7). This effect was transient, and the body temperature returned to normal levels within 3 days. No other significant side effects were observed either systemically or at the injection site (intramuscular delivery in the quadriceps).

Anti-Gag antibodies (Ab) were detected in all the animals after the 2nd vaccination (FIG. 1B), reaching peak responses after the 3rd vaccination in all groups, irrespective of the immunogen used. Responses to the vaccinations were rapid and reached maximal levels one to two weeks after each vaccination. Ab levels showed similar peak responses for the CE (group 1) and gag group (group 2), in agreement with our previous observations with DNA vaccinations (15). There was no difference among the groups up to 8 weeks post vaccination 4 (week 32). The Ab responses were further monitored over time in a subset of 8 macaques (groups 1 and 3). Sustained Gag Ab responses (FIG. 1C) were found with a bi-phasic decline with an initial median 2.4 log decline to week 40 which then plateaued up to week 62 of the follow-up. Together, the data illustrate the induction of robust and durable Ab responses by the CE and CE/CE+Gag mRNA/LNP vaccinations.

Vaccine-induced antigen-specific T cell responses were analyzed in PBMC by flow cytometry upon stimulation with p55Gag and CE peptide pools. Threshold levels of responses were found after 2 vaccinations, while T cell memory (CD95⁺) responses to Gag (FIG. 1D) and CE (FIG. 1E) were detected in the majority of the animals after the 4th vaccination. The response rate for the vaccine-induced T cell immunity was less consistent among animals in the different groups, than the strong humoral responses elicited by the vaccines in all macaques (FIG. 1B). Gag- and CE-specific T cell responses were mediated by both CD4⁺ and CD8⁺ memory T cells (FIGS. 1D and 1E, upper and lower panels). The antigen-specific CD4⁺ T cell responses were compatible with Th1 phenotype (IFN-γ and TNF-α secretion). The animal-to-animal difference in ability to mount distinct (CD4 vs CD8) T cell responses was as expected from outbred macaques. Importantly, despite the overall low level of antigen-specific IFN-γ⁺CD4⁺ T cell responses in blood, remarkable durability of humoral responses was observed (FIG. 1C), supporting the presence of efficient CD4⁺ T helper responses.

Previous studies have shown that DNA vaccination, using an optimized formula including IL-12 DNA as vaccine enhancer, administered by IM injection followed by electroporation induced robust antigen-specific T cells [reviewed in (33, 38)]. Therefore, the T cell responses elicited by the mRNA/LNP vaccine regimen in group 3 were compared to responses obtained from macaques vaccinated with DNA (4 mg dose) expressing the same immunogens [CE prime-CE+gag boost (30)]. This comparison showed lower T cell responses in the mRNA/LNP group targeting Gag (˜40-fold, FIG. 1F, left panel) and CE (˜80-fold, FIG. 1F, right panel) epitopes. It is possible that differences in the dose between the mRNA vs DNA vaccines or the vaccine composition contributed to this.

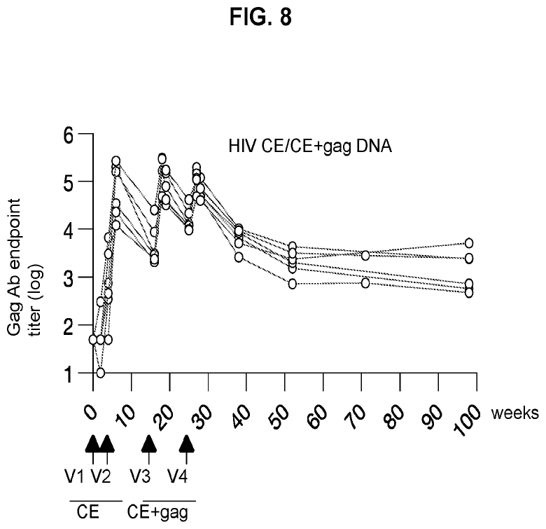

Comparison of vaccine-induced Gag Ab levels in the matching DNA group showed similar kinetics over time (FIG. 8) as the mRNA/LNP group with similar durability (FIG. 1G). Like the mRNA vaccine, the DNA vaccine induced Gag Ab that showed a biphasic decline with an initial median 1.8 log decline over ˜25 weeks, which then plateaued up to week 73 of the follow-up. Long-term durability showed similar sustained Ab levels upon the 4th vaccination in the mRNA (week 62) and DNA (week 73) groups (FIG. 1H).

In conclusion, the HIV mRNA/LNP vaccines induced high durable humoral but low cellular responses, even after 4 vaccinations, in naïve vaccinated macaques. The analogous DNA vaccine induced similar levels of humoral responses but significantly higher cellular responses.

### Example 2: High Dose Gag mRNA/LNP Vaccine in Naïve Macaques

It was tested whether increasing the mRNA/LNP dose from 25 to 100 μg could improve the induction of antigen-specific immune responses (FIG. 2). Macaques (n=5) were immunized twice with a 100 μg dose of the gag mRNA/LNP vaccine (FIG. 2A). After the 2nd vaccination, the immune responses were compared to data from animals in group 2 (see FIG. 1), vaccinated twice with the 25 μg dose.

Anti-Gag Ab were detected in all five animals (FIG. 2B) after the 2nd vaccination. Comparison to the 25 μg dose (FIG. 2C) showed only a slightly higher level (median 2.7 fold) which did not reach significance. These data indicated that increasing the vaccine dose did not provide an additional advantage for the development of humoral responses.

Analysis of the Gag-specific T cells revealed induction of both CD4⁺ and CD8⁺ Gag-specific memory T cell responses (FIG. 2D). A higher response rate (4 of 5 macaques) was found compared to the low-dose group after the 2nd vaccination (FIG. 2E). Total Gag-specific T cell responses were ˜6-fold higher in the 100 μg dose group, reaching up to 0.12% of Gag-specific IFN-γ⁺ T cells. These levels were still significantly lower (median 10-fold; p=0.008, Mann-Whitney) than those obtained upon two gag DNA vaccinations (1 mg dose with IL-12 DNA) administered by IM injection followed by electroporation (FIG. 2E).

Together, vaccination with high dose gag mRNA/LNP vaccines induced similar levels of humoral immune responses but resulted in increased magnitude of cellular immune response in comparison to the low-dose vaccination.

### Example 3: Changes in Plasma Cytokine Levels after mRNA/LNP Vaccination

The cytokine signature induced by the mRNA/LNPs vaccination in the macaques was investigated, as shown in FIGS. 1 and 2 (25 and 100 μg/dose). Plasma was collected at the day of vaccination (Day 1) and over time (Days 2, 4 and 8) after each vaccination, and cytokine analysis was performed using the MSD (Meso Scale Discovery) platform. The plasma levels of the 61 analytes listed in Table 1 were evaluated. The cytokine and chemokine profiles measured overtime after each vaccination were represented in heatmaps, volcano plots and plots of selected analytes (FIGS. 3, 4 and 9). No difference was found among the three low-dose vaccine groups (described in FIG. 1), therefore individual measurements were combined for the subsequent analysis of the 15 animals and were also compared to the 5 animals (described in FIG. 2) that received the high-dose mRNA/LNP vaccine.

TABLE 1
Cytokines and Chemokines (N = 61) tested

Analytes with changes (N = 35)
Eotaxin
Eotaxin-2
Eotaxin-3
Fractalkine
GRO-a
I-TAC
IFN-α2a
IL-12/IL-23p40
IL-15
IL-17A/F
IL-17B
IL-17C
IL-17D
IL-17F
IL-18
IL-1Ra
IL-22*
IL-23
IL-2Ra**
IL-6
IL-7
IL-9*
IP-10
M-CSF
MCP-1
MCP-2
MCP-4
MIF
MIP-1α
MIP-1β
MIP-3β
TPO
TRAIL
VEGF-A
YKL-40
Analytes with minimal or no change
(N = 9)
IL-16
IL-1α
IL-2
MDC
SDF-1a
CTACK
ENA-78
MIP-3α***
FLT3L***
Analytes below detection threshold
(N = 17)
G-CSF
GM-CSF
1-309
IFN-γ
IL-10
IL-12p70
IL-13
IL-17A
IL-1β
IL-4
IL-5
IL-8
MCP-3
MIP-5
TARC
TNF-α
TNF-β
*Analyte below threshold of detection in high-dose vaccine
**Analyte absent from MSD kit used in high dose analysis
***Analyte affected in high-dose mRNA/LNP vaccine group only

The low-dose mRNA/LNP vaccinations were associated with a rapid up-regulation (24 hrs post vaccine administration, D2) of type I IFN (IFN-α2a), IL-15, a cytokine involved in the expansion/survival of cytotoxic memory lymphocytes and NK cells (reviewed in (65)), and IFN-responsive chemokines, such as IP-10/CXCL10 and ITAC/CXCL11 (FIGS. 3A and 3D). A rapid induction of the pro-inflammatory cytokines IL-6 and IL-23 was also observed after each vaccination. This systemic response resulted in the release of different members of the IL-17 family of cytokines (FIGS. 3B, 3D, and 4D), as previously reported (66-68). Down-stream of the immunological IL-23/IL-17 axis are other pro-inflammatory cytokines like IL-22 (69), MCP-1/CCL2 and GROa/CXCL1 (70) which were also increased after each vaccination (FIG. 3D). All mRNA/LNP vaccinations resulted in decreased plasma levels of IL-12/23p40 (FIGS. 3C and 3D), the common chain for the heterodimeric IL-12 and IL-23. This decrease was observed within 24 hours of vaccination irrespective of the mRNA/LNP dose (FIG. 3C). IL-12p70 was also monitored as part of the MSD assay, but its plasma concentration was below the limit of detection.

IL-1Ra, a cytokine with an anti-inflammatory role, was also increased (FIG. 3D, FIG. 4E), as were several other chemokines, such as MIP-3B/CCL19, Eotaxin/CCL11, Eotaxin-3/CCL26, MCP-1/CCL2) and MIP-1a/CCL3, responsible for the recruitment of lymphoid and myeloid cells (FIG. 3D). Cytokine levels peaked on the days after vaccinations and some of the effects induced by vaccination were still detectable at day 4, with persistent elevated levels of chemokines including IP-10/CXCL 10, ITAC/CXCL11, MCP-2, MIP-3B/CCL19, and inflammatory modulators IL-18 and IL-1Ra, which declined to baseline by day 8. The circulating levels of all the affected cytokines returned to baseline by day 8 post vaccination (FIG. 3D).

Subsequent vaccinations resulted in an overall similar cytokine profile, suggesting that the innate responses to the mRNA/LNP vaccine mainly affected the observed signature. Notably, the response magnitude for some analytes (e.g., IP-10/CXCL10, ITAC/CXCL11, IL-17C, IL-17D) was reduced after the 4th vaccination (FIGS. 3 and 4D).

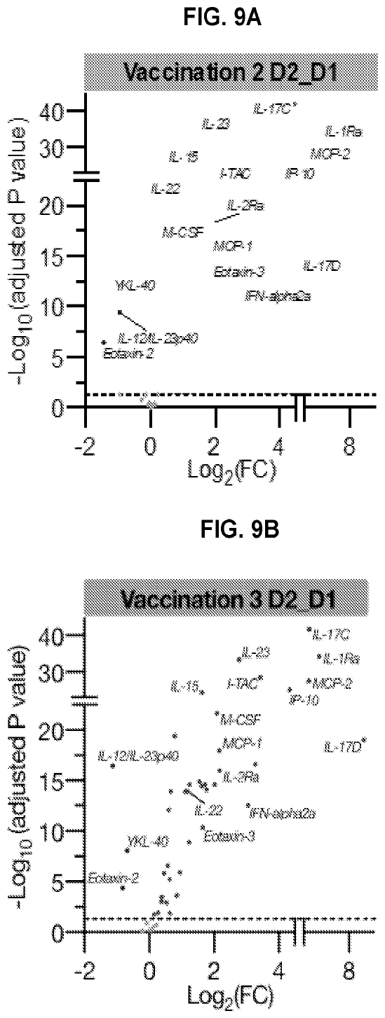

Differential expression analysis was also performed to compare mean log 2 fold change (Log 2FC) of cytokine levels at day 2 to day 1 for all the 15 animals (shown in FIG. 1) receiving the low dose mRNA/LNP vaccine (FIGS. 3E-3F and FIG. 9). The cut-off was set at p<0.05 and was adjusted for multiple comparisons. All vaccinations resulted in a cytokine profile featuring several inflammatory modulators, such as cytokines and chemokines related to the IFN and the IL-17 pathways. The mRNA/LNPs vaccines also negatively impacted the levels of IL-12/23p40, Eotaxin-2 and YKL-40.

Additionally, increasing the vaccine dose showed overall a similar pattern of cytokine/chemokine induction a depicted in heatmaps after the 1st and 2nd vaccination (FIG. 4A) and in volcano plots (FIGS. 4B and 4C). Distinct changes were noted in the response magnitude with analytes being lower (e.g., some members of IL-17 family, IL-23) or higher (e.g., IL-6, IL-1Ra, ITAC/CXCL11). The high dose vaccination was associated with significant higher plasma levels of IL-6 and the chemokines MIP-1B, MIP-3a and ITAC/CXCL11, indicative of the induction of a stronger inflammatory response. Concomitantly, circulating levels of IL-1Ra were ˜10-fold higher in macaques receiving the high dose vaccine in comparison to low dose (FIG. 4D). On contrary, the high vaccine dose was associated with reduced serum levels of IL-23, IL-17A_F, IL-17B, IL-17C, IL-17D (FIGS. 3B and 4D), and monocyte/macrophage chemoattractant M-CSF, MCP-2, MCP-4 (FIGS. 4A-C). Both the high and low dose mRNA/LNP vaccines negatively impacted the levels of IL-12/23p40, YKL-40 and MIF (FIGS. 3 and 4).

The changes in cytokine/chemokine levels did not correlate with changes in immune responses but rather reflected innate activation triggered by the chemical composition of the LNP. Overall, these data identified a cytokine signature induced by the mRNA/LNP vaccine characterized by the induction of inflammation and recruitment of immune cells (both lymphoid and myeloid cells).

### Example 4: DNA Booster Vaccination of the T Cell Responses Primed by Gag mRNA/LNP Vaccination

Next, it was evaluated whether the immune responses elicited by the gag mRNA/LNP vaccination could be boosted by a subsequent single gag DNA vaccination. The study was designed to evaluate the initial response to a heterologous booster vaccination, i.e., using a single gag DNA immunization. The concept of a DNA booster for immune responses induced by mRNA/LNP vaccination was used in lieu of HIV infection-induced responses that cannot be tested in macaques.

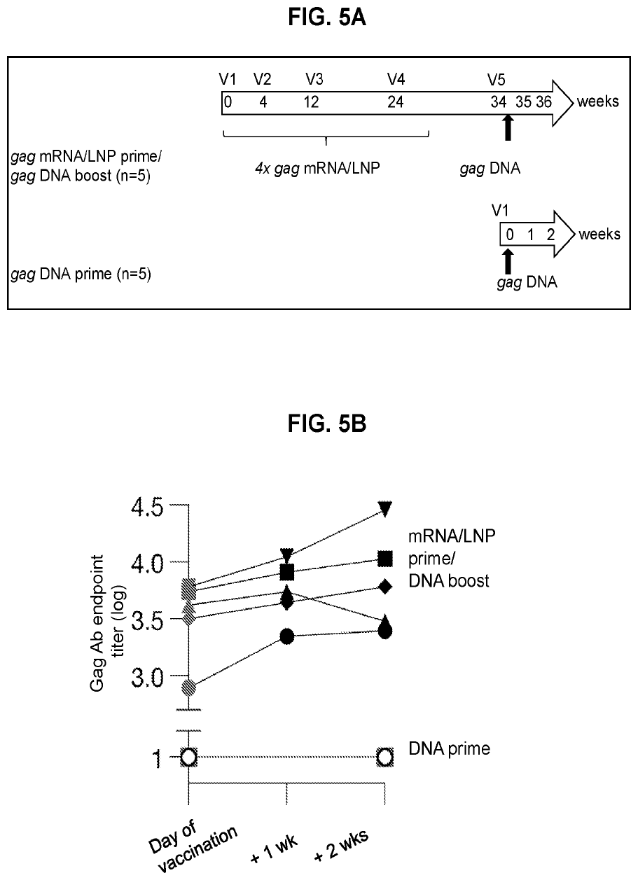

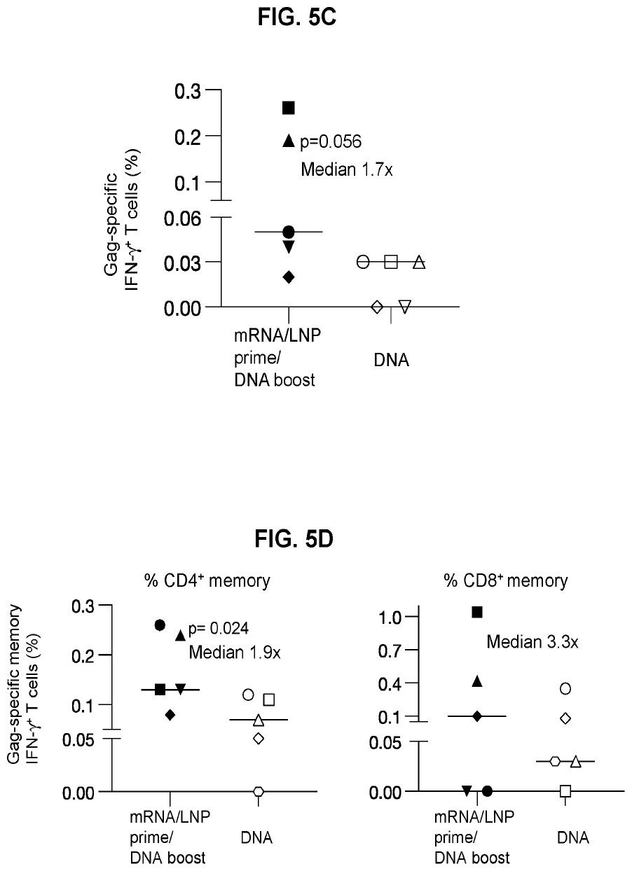

Animals from group 2 (see FIG. 1), which had received prior 4 gag mRNA/LNP vaccinations, were subjected to a single gag DNA booster vaccination (2 mg dose without IL-12 DNA), administered by electroporation, after a 10-week rest (FIG. 5A). To distinguish recall versus de novo responses, five gag-naïve macaques receiving a single DNA vaccination, were included as controls.

The gag mRNA/LNP vaccinated animals showed high levels of Gag antibodies (median 3.6 log, range 2.9-3.8) on the day of vaccination and elicited rapid, anamnestic responses upon a single gag DNA administration with a modest median increase (0.3 log, range 0.1-0.7) (FIG. 5B) over the relatively high pre-existing levels. In contrast, a single gag DNA vaccination of naive macaques did not induce detectable humoral responses within the 2 weeks of follow-up (FIG. 5B). These data showed that mRNA/LNP primed humoral responses could be boosted by a DNA vaccination.

T cell responses were analyzed at 2 weeks post DNA vaccination. Comparison of Gag-specific T cell responses showed a higher response rate and a trend of higher magnitude in the group with pre-existing immunity (FIG. 5C). The Gag-specific responses were significantly higher among the CD4⁺ memory subset (FIG. 5D, left panel; median 0.13% versus 0.07%), likely reflecting their priming with the prior mRNA/LNP vaccination. The difference in CD8⁺ memory responses (median 0.1% versus 0.03%) did not reach significance (FIG. 5D, right panel). Comparison to the magnitude reached at peak upon the 4th mRNA/LNP vaccination only (see FIG. 1D) showed a further increase of T cell memory responses (CD4⁺ increase: median 0.08% to 0.13%; CD8⁺ increase: 0.06% to 0.1%) after the gag DNA boost.

These data show that the low pre-existing T cell responses induced by the mRNA/LNP vaccine were modestly boosted after a single DNA vaccination. The magnitude of antigen-specific memory CD4⁺ T cells upon one single DNA vaccination was significantly higher in macaques previously immunized with mRNA/LNP compared to naïve animals, which supports the concept of an anamnestic response due to priming by the gag mRNA/LNP vaccination.

### Example 5: Gag mRNA/LNP Vaccine Boosts Pre-Existing Humoral and Cellular Immunity Induced by Gag DNA Vaccination

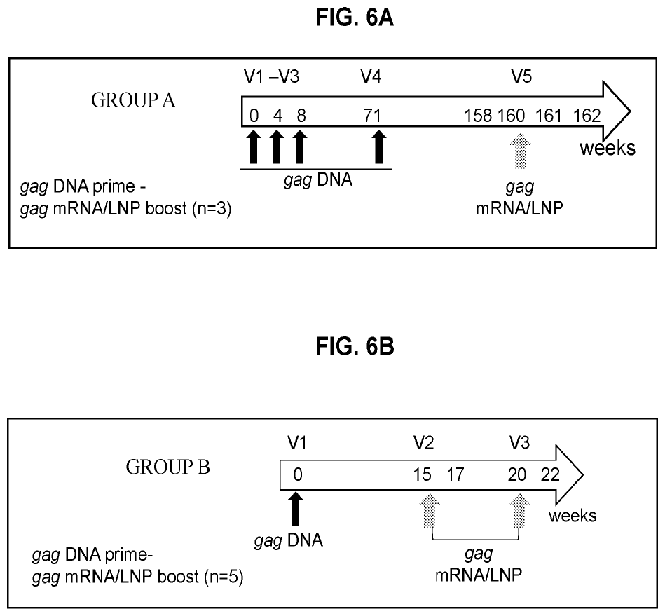

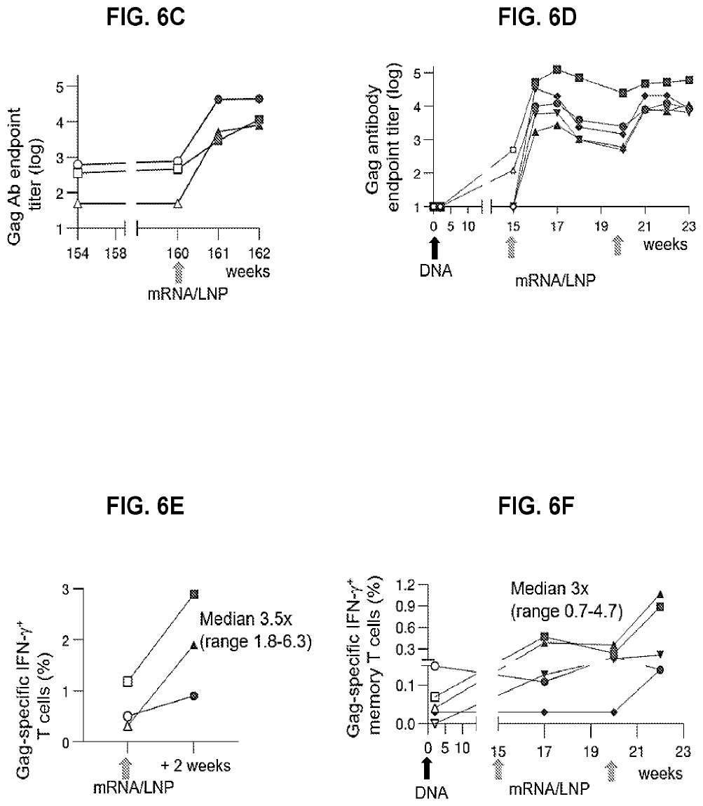

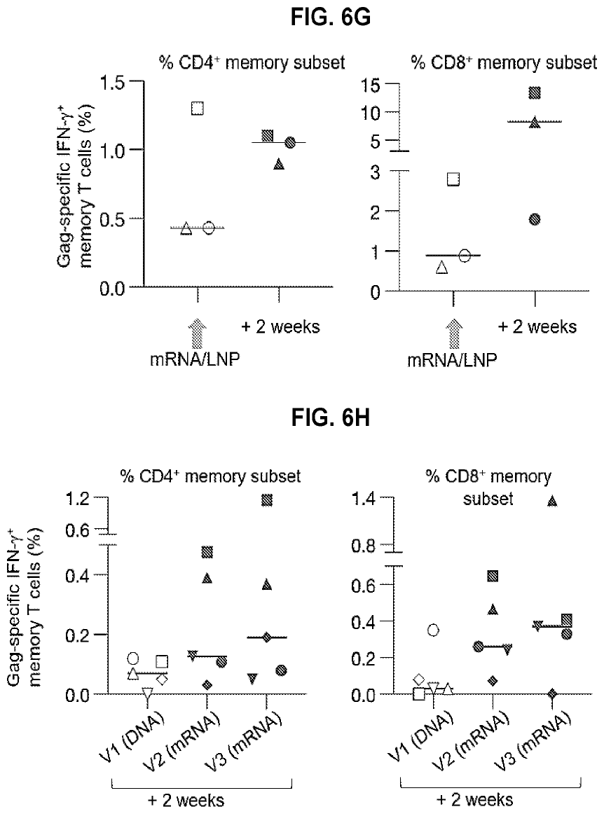

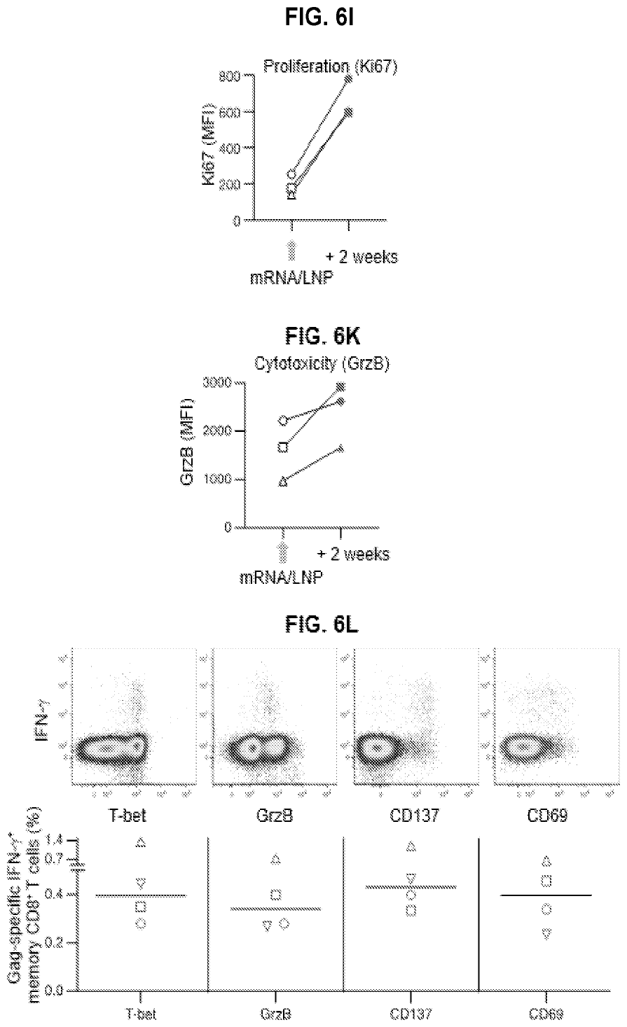

Next the impact of a gag mRNA/LNP booster was examined (FIG. 6) for animals with different levels (high, group A (FIG. 6A) or low, group B (FIG. 6B) of pre-existing immunity. Since macaques cannot be infected by HIV, DNA vaccinated macaques were selected to serve as model for pre-existing immunity. Animals in group A had previously received four HIV gag DNA vaccinations over a period of 3 years (FIG. 6A, n=3), and after a rest of 89 weeks, had a median anti-Gag antibody titer of 2.6 log (range 2.2-2.9 log) at week 158, prior to the mRNA vaccination (FIG. 6C). Animals in group B had received a single HIV gag DNA vaccination (FIG. 6B, n=5) and, after a 15-week rest, their Gag Ab levels were low with only two animals showing titers (2 and 2.7 log, respectively) above the threshold of the assay (FIG. 6D).

Administration of a single gag mRNA/LNP vaccination in group A resulted in a sharp increase (median of 1.7 log) of Gag Ab titers (FIG. 6C). Similarly, a single gag mRNA/LNP booster vaccination in group B resulted in rapid anamnestic humoral response, reaching up to 5 log of anti-Gag Ab titer (FIG. 6D). The antibody response in group B showed a slight contraction 3-5 weeks later and was efficiently boosted again by a 2nd mRNA/LNP vaccination, reaching similar peak Ab levels. Together, the data shown in FIGS. 6C and 6D, surprisingly demonstrated that a single gag mRNA/LNP vaccination was able to induce robust anamnestic humoral responses independent of the magnitude of pre-existing immunity.

Gag-specific T cell responses induced in these two groups of animals were analyzed in PBMC (FIGS. 6E and 6F, respectively). In group A, the priming DNA vaccinations induced Gag-specific T cells that were still detectable 89 weeks after the last vaccination (range 0.3-1.2% of T cells). A single mRNA/LNP vaccination efficiently boosted these responses (2- to 6-fold) in all 3 animals reaching up to 3% of circulating T cells (FIG. 6E). Analysis of the pre-existing memory responses showed ranges of 0.4-1.3% CD4+ and 0.6-2.8% CD8+ memory T cells (FIG. 6G). Two animals showed increases of Gag-specific CD4+ and CD8+ T cells and one animal showed increase only in CD8+ T cells. The responses reached levels up to 1.1% CD4+ and 13.4% memory CD8+ T cells in blood. Characterization of the boosted Gag-specific T cells showed a phenotype of activated cytotoxic T lymphocytes (CTL) with increased proliferative capacity measured by Ki67 expression (FIG. 6I) and increased granzyme B (GrzB) content (FIG. 6K).

Administration of gag mRNA/LNP booster vaccination in animals of group B (FIG. 6B) was also successful in stimulating low pre-existing T cell responses (FIG. 6F). Gag-specific T cell responses increased in all five macaques, with three animals showing responses after the 1st vaccination, and all five animals showing increase after the 2nd mRNA/LNP booster vaccination. The boosted responses were mediated by both CD4+ and CD8+Gag-specific T cells, with a dominant CD8 response (FIG. 6H). The antigen-specific IFN-γ⁺CD8⁺ T cell responses in both groups were characterized by the expression of T-bet and GrzB, reminiscent of a cytotoxic memory phenotype, and the activation markers CD137 and CD69 (FIG. 6L).

Importantly, the gag mRNA/LNP vaccine was more powerful as booster for recall (administered a single time) of cellular immune responses (FIG. 6) than for inducing de novo T cell responses (administered 4 times) (FIGS. 1 and 2). Therefore, the very effective boosting of pre-existing T cell immunity by the HIV gag mRNA/LNP could have general application of this vaccine platform as part of prime-boost regimen. Thus, a heterologous prime/boost regimen aiming to elicit balanced humoral and cellular immunity might be achieved by DNA (or i.e., infection-induced) prime-mRNA boost vaccination.

In the Examples disclosed herein, it was shown that HIV-1 gag mRNA/LNP vaccine regimens induced high antibody responses reaching maximal levels after the 3rd vaccination but were less efficient in the induction of primary T cell responses in naïve rhesus macaques. This dichotomy has already been noticed with other mRNA-based vaccines in certain studies reporting low antigen-specific T cell responses in blood of macaques and humans (35, 50, 52, 54, 55, 58, 59, 71). Although induction of adaptive T cell responses by the CE/gag mRNA/LNP vaccine was low in naïve macaques in comparison to a DNA vaccine regimen, we found persistence and similar magnitude of Gag antibody responses for >62 weeks after the 4th vaccination. These data indicate that despite low levels of the antigen-specific IFN-γ⁺CD4⁺ T cells in the blood, the mRNA/LNP vaccine induced efficient CD4⁺ T helper responses, enabling extended longevity of the humoral responses.

In contrast to de novo responses, the mRNA/LNP booster vaccination of animals with pre-existing Gag-specific T cells resulted in rapid and strong recall T cell responses. The induced T cells showed a Gag-specific cytotoxic effector phenotype characterized by high granzyme B content and T-bet expression, a transcriptional factor associated with Th1 response and cytotoxic CD8+ and NK cells (72). The macaque studies showed rapid and high Ab responses upon a single gag mRNA/LNP booster vaccination, and these Ab responses were of higher magnitude than those elicited by a single low or high dose gag mRNA/LNP vaccination in naïve animals. By analogy, in humans, a single SARS-COV-2 mRNA vaccination [BNT162b2 mRNA (39); CVnCOV (56)] also efficiently boosted antibodies in persons with pre-existing immunity, being more efficient than vaccination of COVID-19-naïve persons (39, 73). In addition, mRNA/LNP vaccination induced CD4⁺ T cell responses against SARS-COV-2 more readily in convalescent patients (74). These data support the conclusion that heterologous vaccine regimens combining e.g., DNA with mRNA/LNPs could be a promising regimen to induce optimal, effective, and balanced humoral and cellular immunity. Specifically, the inclusion of mRNA-based immunogens could be useful in immune therapeutic regimens aiming to treat chronic HIV-1 infection or other pathological conditions to enhance pre-existing immunity.

Cytokines and chemokines are important drivers of inflammation and innate immunity and have a pivotal role in the development and maintenance of adaptive immunity in response to both infection and vaccination. The identification of a cytokine signature could be instrumental for vaccine optimization (75-78). Immune signatures have been reported in different vaccine studies in humans including Yellow fever, HIV-Ade5, HIV ALVAC, SARS-COV-2 BNT162b2 mRNA (39, 79-82). To identify markers associated with vaccination with the gag mRNA/LNP, cytokines and chemokines triggered by prime and boost vaccinations in macaques were studied. It was found that mRNA/LNP vaccinations triggered significant systemic transient (24 hrs) innate cytokine responses characterized by the release of type I IFN, IL-15 and interferon-related chemokines. A decrease in the plasma levels of IL-12/23p40 was also observed after each mRNA vaccination, but, in contrast, an increase in the IL-23 concentration was observed, a cytokine that shares the p40 chain with IL-12. This increase, together with the increase in IL-6, resulted in repeated stimulation of several pro-inflammatory cytokines, especially those from the IL-17 family. The relationship between IL-23 and Th-17 cells is a well-known pro-inflammatory axis (66-68, 83) that is activated in several human diseases.

It was previously reported that SARS-COV-2 BNT162b2 mRNA vaccine in human volunteers induced distinct early (24 hrs) transient cytokine responses featuring IL-15, IFN-γ and IP-10/CXCL10 that also included TNF-α and IL-6, upon booster vaccination (39). In addition, it was reported that the BNT162b2 mRNA vaccine-induced IFN-γ and IL-15 changes correlated with Spike-RBD antibody responses (39), associating these biomarkers with effective development of vaccine-induced humoral responses upon modified mRNA/LNP vaccination. In comparison to the human study, using a different mRNA vaccine platform in macaques, significant increases of IL-15, IP-10/CXCL10 and IL-6 were also found, but the levels of critical components of the signature including IFN-γ and TNF-α were below the threshold of the assay in macaques. It is intriguing that two human vaccine studies with different platforms using BNT 162b2 mRNA COVID-19 (39) and the non-replicating HIV-ALVAC vaccine [expressing HIV Gag, Pro, Env by a non-replicating avian vaccinia vector (canary pox virus) and alum-adjuvanted gp120 protein; (81)] showed induction of cytokines IFN-γ, IL-15 and IP-10/CXCL10. Both IL-15 and IP-10/CXCL 10 were also strongly induced upon gag mRNA/LNP vaccination in macaques. Both IFN-γ and IP-10/CXCL 10 play a role in the IL-15 effects on the immune system (84-86) and a mechanism by which IL-15 indirectly acts on dendritic cells and macrophages/monocytes to induce the secretion of IP-10/CXCL10 via IFN-γ has been reported (87) [reviewed in (65)]. In contrast to the macaque study, the human study did not show detectable levels or changes for the IL-17 chemokine family and IL-23. The underlying reasons to explain such differences includes species (human, macaques); nature of mRNAs (modified versus non-chemically modified); immunogen (SARS-CoV-2 Spike versus HIV Gag based immunogen); and the statistical variation due to the small numbers of macaques enrolled (15 macaques versus 58 human volunteers). Thus, although the macaque study shared some of the chemokine/cytokine markers with the human study, it did not reveal a strong signature correlating to adaptive immune responses. In contrast to the human study with BNT162b2 mRNA which showed stronger innate responses upon the 2nd vaccination (39), the macaque study showed comparable responses upon each vaccination, indicating key differences between the models.

This report shows that the gag mRNA/LNP vaccine induced high and durable antibody responses and low T cell responses in naïve macaques. In comparison, an antigen-matched DNA vaccine induced both strong antibody and T cell responses. Importantly, including a mRNA/LNP booster vaccination in DNA primed macaques greatly augmented potent cytotoxic T cell responses, supporting the potency of the mRNA/LNP vaccine. Therefore, its application in a combination vaccine with other platforms including DNA or as a therapeutic vaccine to stimulate pre-existing immunity is promising.

Having described the subject matter of the disclosure in detail and by reference to specific embodiments thereof, it will be apparent that modifications and variations are possible without departing from the scope of the claimed subject matter. More specifically, although some aspects of the present disclosure are identified herein as particularly advantageous, it is contemplated that the present subject matter is not necessarily limited to these particular aspects of the claimed subject matter.

TABLE 2
LNP-formulated RNA vaccine sequences

HIV gag plasmid DNA 114H
HIV-1 clade B HXB2 gag sequence is in bold and underlined
CCTGGCCATTGCATACGTTGTATCCATATCATAATATGTACATTTATATTGGCTCATGTCCAACATTAC
CGCCATGTTGACATTGATTATTGACTAGTTATTAATAGTAATCAATTACGGGGTCATTAGTTCATAGCC
CATATATGGAGTTCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCCATTGACGTCAAT
GGGTGGAGTATTTACGGTAAACTGCCCACTTGGCAGTACATCAAGTGTATCATATGCCAAGTACGCCCC
CTATTGACGTCAATGATGGTAAATGGCCCGCCTGGCATTATGCCCAGTACATGACCTTATGGGACTTTC
CTACTTGGCAGTACATCTACGTATTAGTCATCGCTATTACCATGGTGATGCGGTTTTGGCAGTACATCA
ATGGGCGTGGATAGCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCATTGACGCAAATGG
GCGGTAGGCGTGTACGGTGGGAGGTCTATATAAGCAGAGCTCGTTTAGTGAACCGTCAGATCGCCTGGA
GACGCCATCCACGCTGTTTTGACCTCCATAGAAGACACCGGGACCGATCCAGCCTCCGCGGGCGCGCGA
AGAAATGGGGGCGCGGGCCTCGGTCCTTAGCGGGGGCGAGTTGGATCGGTGGGAAAAGATCCGCTTGAG
GCCAGGAGGGAAGAAGAAGTACAAGCTAAAGCACATCGTCTGGGCGAGCAGAGAGTTGGAGCGGTTCGC
GGTCAACCCGGGCCTGCTTGAGACATCGGAGGGCTGTCGGCAAATCCTGGGGCAGCTTCAACCGTCCTT
GCAAACGGGCAGCGAGGAGCTTCGATCACTATACAACACTGTAGCAACGCTCTACTGCGTGCACCAGCG
GATCGAGATCAAGGACACGAAGGAGGCTCTTGACAAGATTGAGGAAGAGCAGAACAAGTCCAAGAAGAA
GGCCCAGCAGGCGGCGGCCGACACCGGCCACTCCAACCAAGTATCACAGAACTACCCGATCGTGCAGAA
CATCCAGGGACAGATGGTCCACCAGGCCATCTCCCCACGGACGCTTAACGCGTGGGTCAAAGTAGTGGA
GGAGAAGGCCTTCAGCCCGGAAGTGATCCCCATGTTCTCGGCACTTTCCGAGGGAGCCACCCCGCAGGA
CCTGAACACGATGTTGAACACCGTCGGCGGGCACCAGGCGGCCATGCAGATGCTTAAGGAGACCATCAA
CGAGGAGGCTGCGGAGTGGGACCGGGTCCACCCGGTGCACGCGGGGCCCATCGCGCCGGGCCAGATGAG
AGAGCCGCGGGGATCGGACATCGCGGGAACCACCAGCACCTTGCAGGAGCAAATCGGTTGGATGACTAA
CAACCCGCCAATCCCGGTCGGGGAGATCTACAAGAGATGGATCATCCTCGGGTTGAACAAGATCGTGAG
GATGTACAGCCCGACCAGCATCCTGGACATCCGACAGGGACCGAAGGAGCCGTTCAGAGACTACGTAGA
CCGGTTCTACAAGACTCTCCGGGCGGAGCAGGCGTCGCAGGAGGTCAAGAACTGGATGACGGAGACCTT
GTTGGTCCAGAACGCGAACCCGGACTGCAAGACCATCCTGAAGGCTCTCGGCCCGGCGGCGACGTTGGA
AGAGATGATGACGGCGTGCCAGGGAGTCGGGGGACCCGGCCACAAGGCGCGGGTCTTGGCCGAGGCGAT
GAGCCAAGTGACGAACTCGGCGACGATCATGATGCAGCGGGGCAACTTCCGGAACCAGCGGAAGATCGT
CAAGTGCTTCAACTGTGGCAAGGAGGGACACACCGCCAGGAACTGCCGGGCCCCCCGGAAGAAGGGCTG
CTGGAAGTGCGGAAAGGAGGGGCACCAAATGAAGGACTGCACGGAGCGGCAGGCGAATTTCCTCGGGAA
GATCTGGCCGTCCTACAAGGGGCGGCCAGGGAACTTTCTGCAAAGCCGGCCGGAGCCGACCGCCCCGCC
GGAGGAGTCCTTTCGGTCCGGGGTCGAGACGACCACGCCCCCTCAGAAGCAAGAGCCCATCGACAAGGA
GTTGTACCCTCTTACCTCCCTCCGGTCGCTCTTCGGCAACGACCCGTCCTCGCAATGATAAGAATTCGA
GCTCGGTACGATCCAGATCTGCTGTGCCTTCTAGTTGCCAGCCATCTGTTGTTTGCCCCTCCCCCGTGC
CTTCCTTGACCCTGGAAGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAAGGGGGAGGATTGGGAAG
ACAATAGCAGGCATGCTGGGGATGCGGTGGGCTCTATGGGTACCCAGGTGCTGAAGAATTGACCCGGTT
CCTCCTGGGCCAGAAAGAAGCAGGCACATCCCCTTCTCTGTGACACACCCTGTCCACGCCCCTGGTTCT
TAGTTCCAGCCCCACTCATAGGACACTCATAGCTCAGGAGGGCTCCGCCTTCAATCCCACCCGCTAAAG
TACTTGGAGCGGTCTCTCCCTCCCTCATCAGCCCACCAAACCAAACCTAGCCTCCAAGAGTGGGAAGAA
ATTAAAGCAAGATAGGCTATTAAGTGCAGAGGGAGAGAAAATGCCTCCAACATGTGAGGAAGTAATGAG
AGAAATCATAGAATTTCTTCCGCTTCCTCGCTCACTGACTCGCTGCGCTCGGTCGTTCGGCTGCGGCGA
GCGGTATCAGCTCACTCAAAGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGACAGGACTA
TAAAGATACCAGGCGTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCATAGCTCACGCTGTAGGTATCTC
AGTTCGGTGTAGGTCGTTCGCTCCAAGCTGGGCTGTGTGCACGAACCCCCCGTTCAGCCCGACCGCTGC
GCCTTATCCGGTAACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAAGTGGTGGCCTAAC
TACGGCTACACTAGAAGAGACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTTTGTTTGCAAGCAGCA
GATTACGCGCAGAAAAAAAGGATCTCAAGAAGATCCTTTGATCTTTTCTACGGGGTCTGACGCTCAGTG
GAACGAAAACTCACGTTAAGGGATTTTGGTCATGAGATTATCAAAAAGGATCTTCACCTAGATCCTTTT
AAATTAAAAATGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCCTGACTCGGGGG
GGGGGGGCGCTGAGGTCTGCCTCGTGAAGAAGGTGTTGCTGACTCATACCAGGCCTGAATCGCCCCATC
ATCCAGCCAGAAAGTGAGGGAGCCACGGTTGATGAGAGCTTTGTTGTAGGTGGACCAGTTGGTGATTTT
GAACTTTTGCTTTGCCACGGAACGGTCTGCGTTGTCGGGAAGATGCGTGATCTGATCCTTCAACTCAGC
AAAAGTTCGATTTATTCAACAAAGCCGCCGTCCCGTCAAGTCAGCGTAATGCTCTGCCAGTGTTACAAC
CAATTAACCAATTCTGATTAGAAAAACTCATCGAGCATCAAATGAAACTGCAATTTATTCATATCAGGA
TTATCAATACCATATTTTTGAAAAAGCCGTTTCTGTAATGAAGGAGAAAACTCACCGAGGCAGTTCCAT
AGGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGGC
AAAAGCTTATGCATTTCTTTCCAGACTTGTTCAACAGGCCAGCCATTACGCTCGTCATCAAAATCACTC
GCATCAACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAATATTTTCA
CCTGAATCAGGATATTCTTCTAATACCTGGAATGCTGTTTTCCCGGGGATCGCAGTGGTGAGTAACCAT
GCATCATCAGGAGTACGGATAAAATGCTTGATGGTCGGAAGAGGCATAAATTCCGTCAGCCAGTTTAGT
CTGACCATCTCATCTGTAACATCATTGGCAACGCTACCTTTGCCATGTTTCAGAAACAACTCTGGCGCA
TCGGGCTTCCCATACAATCGATAGATTGTCGCACCTGATTGCCCGACATTATCGCGAGCCCATTTATAC
CCATATAAATCAGCATCCATGTTGGAATTTAATCGCGGCCTCGAGCAAGACGTTTCCCGTTGAATATGG
CTCATAACACCCCTTGTATTACTGTTTATGTAAGCAGACAGTTTTATTGTTCATGATGATATATTTTTA
TCTTGTGCAATGTAACATCAGAGATTTTGAGACACAACGTGGCTTTCCCCCCCCCCCCATTATTGAAGC
ATTTATCAGGGTTATTGTCTCATGAGCGGATACATATTTGAATGTATTTAGAAAAATAAACAAATAGGG
GTTCCGCGCACATTTCCCCGAAAAGTGCCACCTGACGTCTAAGAAACCATTATTATCATGACATTAACC
TATAAAAATAGGCGTATCACGAGGCCCTTTCGTCTCGCGCGTTTCGGTGATGACGGTGAAAACCTCTGA
CACATGCAGCTCCCGGAGACGGTCACAGCTTGTCTGTAAGCGGATGCCGGGAGCAGACAAGCCCGTCAG
GGCGCGTCAGCGGGTGTTGGCGGGTGTCGGGGCTGGCTTAACTATGCGGCATCAGAGCAGATTGTACTG
AGAGTGCACCATATGCGGTGTGAAATACCGCACAGATGCGTAAGGAGAAAATACCGCATCAGATTGGCT
ATTGG (SEQ ID NO: 01)
HIV Gag (Clade B, HXB2) protein
HIV Gag B, HXB2) S protein
M G A R A S V L S G G E L A R W E K I R L R P G
G K K K Y K L K H I V W A S R E L E R F A V N P
G L L E T S E G C R Q I L G Q L Q P S L Q T G S
E E L R S L Y N T V A T L Y C V H Q R I E I K D
T K E A L D K I E E E Q N K S K K K A Q Q A A A
D T G H S N Q V S Q N Y P I V Q N I Q G Q M V H
Q A I S P R T L N A W V K V V E E K A F S P E V
I P M F S A L S E G A T P Q D L N T M L N T V G
G H Q A A M Q M L K E T I N E E A A E W D R V H
P V H A G P I A P G Q M R E P R G S D I A G T T
S T L Q E Q I G W M T N N P P I P V G E I Y K R
W I I L G L N K I V R M Y S P T S I L D I R Q G
P K E P F R D Y V D R F Y K T L R A E Q A S Q E
V K N W M T E T L L V Q N A N P D C K T I L K A
L G P A A T L E E M M T A C Q G V G G P G H K A
R V L A E A M S Q V T N S A T I M M Q R G N F R
N Q R K I V K C F N C G K E G H T A R N C R A P
R K K G C W K C G K E G H Q M K D C T E R Q A N
F L G K I W P S Y K G R P G N F L Q S R P E P T
A P P E E S F R S G V E T T T P P Q K Q E P I D
K E L Y P L T S L R S L F G N D P S S Q
(SEQ ID NO: 02)
p24CE1/2 plasmid 306H (CE1 and CE2 gene)
CCTGGCCATTGCATACGTTGTATCCATATCATAATATGTACATTTATATTGGCTCATGTCCAACATTAC
CGCCATGTTGACATTGATTATTGACTAGTTATTAATAGTAATCAATTACGGGGTCATTAGTTCATAGCC
CATATATGGAGTTCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCCATTGACGTCAAT
GGGTGGAGTATTTACGGTAAACTGCCCACTTGGCAGTACATCAAGTGTATCATATGCCAAGTACGCCCC
CTATTGACGTCAATGATGGTAAATGGCCCGCCTGGCATTATGCCCAGTACATGACCTTATGGGACTTTC
CTACTTGGCAGTACATCTACGTATTAGTCATCGCTATTACCATGGTGATGCGGTTTTGGCAGTACATCA
ATGGGCGTGGATAGCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCATTGACGCAAATGG
GCGGTAGGCGTGTACGGTGGGAGGTCTATATAAGCAGAGCTCGTTTAGTGAACCGTCAGATCGCCTGGA
GACGCCATCCACGCTGTTTTGACCTCCATAGAAGACACCGGGACCGATCCAGCCTCCGCGGGCGCGCGT
CGACAAGAAATGTGGCTCCAGAGCCTGCTACTCCTGGGGACGGTGGCCTGCAGCATCTCGGTCATCCCG
ATGTTCTCGGCGCTCAGCGAGGGAGCGACGCCGCAGGACCTGAACGCGGCCGTCGGAGGTCACCAGGCA
GCGATGCAGATGCTGAAGGACACGATCAACGAGGAGGCGGCCGAGTGGGACCGGGCGGCAGCCGAGCCA
CGCGGTTCCGACATCGCGGGCACCACCTCGACGCTCCAGGAGCAGATCGGGTGGGCCGCAGCTAAGCGC
TGGATCATCCTCGGGCTGAACAAGATCGTCCGGATGTACAGCCCGACGTCGATCGCTGCTAAGTACGTT
GACCGGTTCTACAAGACCCTGAGGGCCGAGCAGGCGGCCGGACTGGAGGAGATGATGACCGCGTGCCAG
GGGGTCGGTGGACCAGGGCACAAGGCCGCGATCTCGCCGCGCACGCTGAACGCGTGGGTGAAGGTCTGA
TAAGAATTCGCTAGCGGCGCGCCAGATCTGATATCGGATCTGCTGTGCCTTCTAGTTGCCAGCCATCTG
TTGTTTGCCCCTCCCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGGGCTCTATGGGTACCCAGG
TGCTGAAGAATTGACCCGGTTCCTCCTGGGCCAGAAAGAAGCAGGCACATCCCCTTCTCTGTGACACAC
CCTGTCCACGCCCCTGGTTCTTAGTTCCAGCCCCACTCATAGGACACTCATAGCTCAGGAGGGCTCCGC
CTTCAATCCCACCCGCTAAAGTACTTGGAGCGGTCTCTCCCTCCCTCATCAGCCCACCAAACCAAACCT
AGCCTCCAAGAGTGGGAAGAAATTAAAGCAAGATAGGCTATTAAGTGCAGAGGGAGAGAAAATGCCTCC
AACATGTGAGGAAGTAATGAGAGAAATCATAGAATTTCTTCCGCTTCCTCGCTCACTGACTCGCTGCGC
TCGGTCGTTCGGCTGCGGCGAGCGGTATCAGCTCACTCAAAGGCGGTAATACGGTTATCCACAGAATCA
GGGGATAACGCAGGAAAGAACATGTGAGCAAAAGGCCAGCAAAAGGCCAGGAACCGTAAAAAGGCCGCG
TTGCTGGCGTTTTTCCATAGGCTCCGCCCCCCTGACGAGCATCACAAAAATCGACGCTCAAGTCAGAGG
TGGCGAAACCCGACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTGGCGCTTTCTCAT
AGCTCACGCTGTAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGGTAAGACACGAC
TTATCGCCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTTGATCTTTTCT
ACGGGGTCTGACGCTCAGTGGAACGAAAACTCACGTTAAGGGATTTTGGTCATGAGATTATCAAAAAGG
ATCTTCACCTAGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATTTCGTTCATCC
ATAGTTGCCTGACTCGGGGGGGGGGGGCGCTGAGGTCTGCCTCGTGAAGAAGGTGTTGCTGACTCATAC
CAGGCCTGAATCGCCCCATCATCCAGCCAGAAAGTGAGGGAGCCACGGTTGATGAGAGCTTTGTTGTAG
GTGGACCAGTTGGTGATTTTGAACTTTTGCTTTGCCACGGAACGGTCTGCGTTGTCGGGAAGATGCGTG
ATCTGATCCTTCAACTCAGCAAAAGTTCGATTTATTCAACAAAGCCGCCGTCCCGTCAAGTCAGCGTAA
TGCTCTGCCAGTGTTACAACCAATTAACCAATTCTGATTAGAAAAACTCATCGAGCATCAAATGAAACT
GCAATTTATTCATATCAGGATTATCAATACCATATTTTTGAAAAAGCCGTTTCTGTAATGAAGGAGAAA
ACTCACCGAGGCAGTTCCATAGGATGGCAAGATCCTGGTATCGGTCTGCGATTCCGACTCGTCCAACAT
CAATACAACCTATTAATTTCCCCTCGTCAAAAATAAGGTTATCAAGTGAGAAATCACCATGAGTGACGA
CTGAATCCGGTGAGAATGGCAAAAGCTTATGCATTTCTTTCCAGACTTGTTCAACAGGCCAGCCATTAC
GCTCGTCATCAAAATCACTCGCATCAACCAAACCGTTATTCATTCGTGATTGCGCCTGAGCGAGACGAA
ATACGCGATCGCTGTTAAAAGGACAATTACAAACAGGAATCGAATGCAACCGGCGCAGGAACACTGCCA
GCGCATCAACAATATTTTCACCTGAATCAGGATATTCTTCTAATACCTGGAATGCTGTTTTCCCGGGGA
TCGCAGTGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCCATGTT
TCAGAAACAACTCTGGCGCATCGGGCTTCCCATACAATCGATAGATTGTCGCACCTGATTGCCCGACAT
TATCGCGAGCCCATTTATACCCATATAAATCAGCATCCATGTTGGAATTTAATCGCGGCCTCGAGCAAG
ACGTTTCCCGTTGAATATGGCTCATAACACCCCTTGTATTACTGTTTATGTAAGCAGACAGTTTTATTG
TTCATGATGATATATTTTTATCTTGTGCAATGTAACATCAGAGATTTTGAGACACAACGTGGATCATCC
AGACATGATAAGATACATTGATGAGTTTGGACAAACCACAACTAGAATGCAGTGAAAAAAATGCTTTAT
TTGTGAAATTTGTGATGCTATTGCTTTATTTGTAACCATTATAAGCTGCAATAAACAAGTTAACAACAA
CAATTGCATTCATTTTATGTTTCAGGTTCAGGGGGAGGTGTGGGAGGTTTTTTAAAGCAAGTAAAACCT
CTACAAATGTGGTATGGCTGATTATGATCGTCGAGGATCCGGCTTATCAGACCTTCACCCAGGCGTTGA
GGGTGCGAGGCGAGAGGGCCGCCTTGTGCGACGGTCCTCCGACTCCCTGGCAGGCTGTCATCATCTCCT
CGAGACCCGCGGCCTGCTCTGCCCTCAGCGTCTTGAAGAAGCGGTCTACGTATTTGGCCGCGATGCTGA
CTGGGCTGTACATCCTGACGATCTTGTTGAGGCCCAGGATGATCCAGCGCTTGGCTGCAGCCCAGGCGA
TCTGCTCCTGGAGGGTGCTGGTCGTGCCTGCGATGTCGCTACCCCTTGGCTCAGCTGCTGCCCTGTCCC
ACTCGGCTGCCTCCTCGTTGATGGTCTCCTTGAGCATCTGCATTGCCGCCTGGTGTCCACCGACCGCGG
CGTTGAGGTCCTGCGGTGTCGCACCCTCACTGAGTGCGGTGAACATGGGGATGACCGAGATCGAGCACG
CCACGGTCCCGAGTAGCAGGAGCGACTGCAGCCACATTTCTTGCCGTTTAAACGTCGACAGATCCAAAC
GCTCCTCCGACGTCCCCAGGCAGAATGGCGGTTCCCTAAACGAGCATTGCTTATATAGACCTCCCATTA
GGCACGCCTACCGCCCATTTACGTCAATGGAACGCCCATTTGCGTCATTGCCCCTCCCCATTGACGTCA
ATGGGGATGTACTTGGCAGCCATCGCGGGCCATTTACCGCCATTGACGTCAATGGGAGTACTGCCAATG
TACCCTGGCGTACTTCCAATAGTAATGTACTTGCCAAGTTACTATTAATAGATATTGATGTACTGCCAA
GTGGGCCATTTACCGTCATTGACGTCAATAGGGGGCGTGAGAACGGATATGAATGGGCAATGAGCCATC
CCATTGACGTCAATGGTGGGTGGTCCTATTGACGTCAATGGGCATTGAGCCAGGGGGGCCATTTACCGT
AATTGACGTCAATGGGGGAGGCGCCATATACGTCAATAGGACCGCCCATATGACGTCAATAGGTAAGAC
CATGAGGCCCTTTCGTCTCGCGCGTTTCGGTGATGACGGTGAAAACCTCTGACACATGCAGCTCCCGGA
GACGGTCACAGCTTGTCTGTAAGCGGATGCCGGGAGCAGACAAGCCCGTCAGGGCGCGTCAGCGGGTGT
TGGCGGGTGTCGGGGCTGGCTTAACTATGCGGCATCAGAGCAGATTGTACTGAGAGTGCACCATATGCG
GTGTGAAATACCGCACAGATGCGTAAGGAGAAAATACCGCATCAGATTGGCTATTGG
(SEQ ID NO: 03)
p24CE1
MWLQSLLLLGTVACSISVIPMFSALSEGATPQDLNAAVGGHQAAMQMLKDTINEEAAEWDRAAAEPRGS
DIAGTTSTLQEQIGWAAAKRWIILGLNKIVRMYSPTSIAAKYVDRFYKTLRAEQAAGLEEMMTACQGVG
GPGHKAAISPRTLNAWVKV (SEQ ID NO: 04)
p24CE2
MWLQSLLLLGTVACSISVIPMFTALSEGATPQDLNAAVGGHQAAMQMLKETINEEAAEWDRAAAEPRGS
DIAGTTSTLQEQIAWAAAKRWIILGLNKIVRMYSPVSIAAKYVDRFFKTLRAEQAAGLEEMMTACQGVG
GPSHKAALSPRTLNAWVKV (SEQ ID NO: 05)

### REFERENCES

1. Churchill M J, Deeks S G, Margolis D M, Siliciano R F, Swanstrom R. HIV reservoirs: what, where and how to target them. Nat Rev Microbiol. (2016); 14(1):55-60. doi:10.1038/nrmicro.2015.5.
2. Martinez-Picado J, Deeks S G. Persistent HIV-1 replication during antiretroviral therapy. Curr Opin HIV AIDS. (2016); 11(4):417-23. doi:10.1097/COH.0000000000000287.
3. Cohn L B, Chomont N, Deeks S G. The Biology of the HIV-1 Latent Reservoir and Implications for Cure Strategies. Cell Host Microbe. (2020); 27(4):519-530. Epub 2020 Apr. 10. doi:10.1016/j.chom.2020.03.014.
4. Fromentin R, Chomont N. HIV persistence in subsets of CD4+ T cells: 50 shades of reservoirs. Semin Immunol. (2021); 51:101438. Epub 2020 Dec. 5. doi:10.1016/j.smim.2020.101438.
5. Chen Z, Julg B. Therapeutic Vaccines for the Treatment of HIV. Trans/Res. (2020); 223:61-75. Epub 2020 May 22. doi:10.1016/j.trsl.2020.04.008.
6. Ward A R, Mota T M, Jones R B. Immunological approaches to HIV cure. Semin Immunol. (2021); 51:101412. Epub 2020 Sep. 29. doi:10.1016/j.smim.2020.101412.
7. Collins D R, Gaiha G D, Walker B D. CD8(+) T cells in HIV control, cure and prevention. Nat Rev Immunol. (2020); 20(8):471-482. Epub 2020 Feb. 14. doi:10.1038/s41577-020-0274-9.
8. Jones R B, Walker B D. HIV-specific CD8(+) T cells and HIV eradication. J Clin Invest. (2016); 126(2):455-63. Epub 2016 Jan. 6. doi:10.1172/JCI80566.
9. Shan L, Deng K, Shroff N S, Durand C M, Rabi S A, Yang H C, Zhang H, Margolick J B, Blankson J N, Siliciano R F. Stimulation of HIV-1-specific cytolytic T lymphocytes facilitates elimination of latent viral reservoir after virus reactivation. Immunity. (2012); 36(3):491-501. Epub 2012 Mar. 13. doi:10.1016/j.immuni.2012.01.014.
10. Mothe B, Brander C. Considerations for successful therapeutic immunization in HIV cure. Curr Opin HIV AIDS. (2021); 16(5):257-261. Epub 2021 Jul. 27. doi:10.1097/COH.0000000000000696.
11. Hanke T. Conserved immunogens in prime-boost strategies for the next-generation HIV-1 vaccines. Expert Opin Biol Ther. (2014); 14:601-16. Epub 2014 Feb. 5. doi:10.1517/14712598.2014.885946.
12. Rogan D C, Connors M. Immunologic Control of HIV-1: What Have We Learned and Can We Induce It? Curr HIV/AIDS Rep. (2021); 18(3):211-220. Epub 2021 Mar. 13. doi:10.1007/s11904-021-00545-2.
13. Patel S, Jones R B, Nixon D F, Bollard C M. T-cell therapies for HIV: Preclinical successes and current clinical strategies. Cytotherapy. (2016); 18(8):931-942. Epub 2016 Jun. 7. doi:10.1016/j.jcyt.2016.04.007.
14. Mothe B, Hu X, Llano A, Rosati M, Olvera A, Kulkarni V, Valentin A, Alicea C, Pilkington G R, Sardesai N Y, Rocafort M, Crespo M, Carrillo J, Marco A, Mullins J I, Dorrell L, Hanke T, Clotet B, Pavlakis G N, Felber B K, Brander C. A Human Immune Data-Informed Vaccine Concept Elicits Strong and Broad T-cell Specificities Associated with HIV-1 Control in Mice and Macaques. J Transl Med. (2015); 13:60.
15. Kulkarni V, Valentin A, Rosati M, Rolland M, Mullins J I, Pavlakis G N, Felber B K. HIV-1 Conserved Elements p24C E DNA Vaccine Induces Humoral Immune Responses with Broad Epitope Recognition in Macaques. PLOS One. (2014); 9 e111085.
16. Kulkarni V, Valentin A, Rosati M, Alicea C, Singh A K, Jalah R, Broderick K E, Sardesai N Y, Le Gall S, Mothe B, Brander C, Rolland M, Mullins J I, Pavlakis G N, Felber B K. Altered Response Hierarchy and Increased T-cell Breadth upon HIV-1 Conserved Element DNA Vaccination in Macaques. PLOS One. (2014); 9:e86254 .
17. Garcia-Bates T M, Palma M L, Anderko R R, Hsu D C, Ananworanich J, Korber B T, Gaiha G D, Phanuphak N, Thomas R, Tovanabutra S, Walker B D, Mellors J W, Piazza P A, Kroon E, Riddler S A, Michael N L, Rinaldo C R, Mailliard R B, I4C, Groups RVS. Dendritic cells focus CTL responses toward highly conserved and topologically important HIV-1 epitopes. EBioMedicine. (2021); 63:103175. Epub 2021 Jan. 16. doi:10.1016/j.ebiom.2020.103175.
18. Borthwick N, Ahmed T, Ondondo B, Hayes P, Rose A, Ebrahimsa U, Hayton E J, Black A, Bridgeman A, Rosario M, Hill A V, Berrie E, Moyle S, Frahm N, Cox J, Colloca S, Nicosia A, Gilmour J, McMichael A J, Dorrell L, Hanke T. Vaccine-elicited Human T Cells Recognizing Conserved Protein Regions Inhibit HIV-1. Mol Ther. (2014); 22(2):464-75. Epub 2013 Oct. 30. doi:10.1038/mt.2013.248.
19. Letourneau S, Im E J, Mashishi T, Brereton C, Bridgeman A, Yang H, Dorrell L, Dong T, Korber B, McMichael A J, Hanke T. Design and pre-clinical evaluation of a universal HIV-1 vaccine. PLOS One. (2007); 2(10):e984. eng. Epub 2007 Oct. 4. doi:10.1371/journal.pone.0000984.
20. Ondondo B, Murakoshi H, Clutton G, Abdul-Jawad S, Wee E G, Gatanaga H, Oka S, McMichael A J, Takiguchi M, Korber B, Hanke T. Novel Conserved-region T-cell Mosaic Vaccine With High Global HIV-1 Coverage Is Recognized by Protective Responses in Untreated Infection. Mol Ther. (2016); 24:832-42. doi:10.1038/mt.2016.3.
21. Barouch D H, Stephenson K E, Borducchi E N, Smith K, Stanley K, McNally A G, Liu J, Abbink P, Maxfield L F, Seaman M S, Dugast A S, Alter G, Ferguson M, Li W, Earl P L, Moss B, Giorgi E E, Szinger J J, Eller L A, Billings E A, Rao M, Tovanabutra S, Sanders-Buell E, Weijtens M, Pau M G, Schuitemaker H, Robb M L, Kim J H, Korber B T, Michael N L. Protective efficacy of a global HIV-1 mosaic vaccine against heterologous SHIV challenges in rhesus monkeys. Cell. (2013); 155(3):531-9. Epub 2013 Nov. 19. doi:10.1016/j.cell.2013.09.061.
22. Barouch D H, O'Brien K L, Simmons N L, King S L, Abbink P, Maxfield L F, Sun Y H, La Porte A, Riggs A M, Lynch D M, Clark S L, Backus K, Perry J R, Seaman M S, Carville A, Mansfield K G, Szinger J J, Fischer W, Muldoon M, Korber B. Mosaic HIV-1 vaccines expand the breadth and depth of cellular immune responses in rhesus monkeys. Nat Med. (2010); 16(3):319-23. eng. Epub 2010 Feb. 23. doi: nm.2089 [pii]10.1038/nm.2089.
23. Yan J, Corbitt N, Pankhong P, Shin T, Khan A, Sardesai N Y, Weiner D B. Immunogenicity of a novel engineered HIV-1 clade C synthetic consensus-based envelope DNA vaccine. Vaccine. (2011); 29(41):7173-81. eng. Epub 2011 Jun. 10. doi:S0264-410X(11)00823-1 [pii] 10.1016/j.vaccine.2011.05.076.
24. Gaiha G D, Rossin E J, Urbach J, Landeros C, Collins D R, Nwonu C, Muzhingi I, Anahtar M N, Waring O M, Piechocka-Trocha A. Waring M, Worrall D P. Ghebremichael M S, Newman R M, Power K A, Allen T M, Chodosh J, Walker B D. Structural topology defines protective CD8(+) T cell epitopes in the HIV proteome. Science. (2019); 364(6439):480-484. Epub 2019 May 3. doi:10.1126/science.aav5095.
25. Rolland M, Manocheewa S, Swain J V, Lanxon-Cookson E C, Kim M, Westfall D H, Larsen B B, Gilbert P B, Mullins J I. HIV-1 conserved-element vaccines: relationship between sequence conservation and replicative capacity. J Virol. (2013); 87(10):5461-7. doi:10.1128/JVI.03033-12.
26. Manocheewa S, Swain J V, Lanxon-Cookson E, Rolland M, Mullins J I. Fitness costs of mutations at the HIV-1 capsid hexamerization interface. PLOS One. (2013); 8(6):e66065. doi:10.1371/journal.pone.0066065.
27. Liu Y, Rao U, McClure J, Konopa P, Manocheewa S, Kim M, Chen L, Troyer R M, Tebit D M, Holte S, Arts E J, Mullins J I. Impact of mutations in highly conserved amino acids of the HIV-1 Gag-p24 and Env-gp120 proteins on viral replication in different genetic backgrounds. PLOS One. (2014); 9(4):e94240. doi:10.1371/journal.pone.0094240.
28. Liu Y, McNevin J, Zhao H, Tebit D M, Troyer R M, McSweyn M, Ghosh A K, Shriner D, Arts E J, McElrath M J, Mullins J I. Evolution of human immunodeficiency virus type 1 cytotoxic T-lymphocyte epitopes: fitness-balanced escape. J Virol. (2007); 81(22):12179-88. eng. Epub 2007 Aug. 31. doi: JVI.01277-07 [pii]10.1128/JVI.01277-07.
29. Kulkarni V, Rosati M, Valentin A, Ganneru B, Singh A K, Yan J, Rolland M, Alicea C, Beach R K, Zhang G M, Le Gall S, Broderick K E, Sardesai N Y, Heckerman D, Mothe B, Brander C, Weiner D B, Mullins J I, Pavlakis G N, Felber B K. HIV-1 p24^{gag} derived conserved element DNA vaccine increases the breadth of immune response in mice. PLos One. (2013); 8:e60245
30. Hu X, Valentin A, Dayton F, Kulkarni V, Alicea A, Rosati M, Chowdhury B, Gautam R, Broderick K E, Sardesai N Y, Martin M A, Mullins J I, Pavlakis G N, Felber B K. DNA Prime-boost Vaccine Regimen to Increase Breadth, Magnitude, and Cytotoxicity of the Cellular Immune Responses to Subdominant Gag Epitopes of SIV and HIV J Immunol. (2016); 197:3999-4013.
31. Hu X, Lu Z, Valentin A, Rosati M, Broderick K E, Sardesai N Y, Marx P A, Mullins J I, Pavlakis G N, Felber B K. Gag and env conserved element C E DNA vaccines elicit broad cytotoxic T cell responses targeting subdominant epitopes of HIV and SIV Able to recognize virus-infected cells in macaques. Hum Vaccin Immunother. (2018); 14(9):2163-2177. Epub 2018 Jun. 26. doi:10.1080/21645515.2018.1489949.
32. Rauch S, Jasny E, Schmidt K E, Petsch B. New Vaccine Technologies to Combat Outbreak Situations. Front Immunol. (2018); 9:1963. Epub 2018 Oct. 5. doi:10.3389/fimmu.2018.01963.
33. Felber B K, Valentin A, Rosati M, Bergamaschi C, Pavlakis G N. HIV DNA Vaccine: Stepwise improvements make a difference. Vaccines. (2014); 2:354-379.
34. Pardi N, Weissman D. Nucleoside Modified mRNA Vaccines for Infectious Diseases. Methods Mol Biol. (2017); 1499:109-121. doi:10.1007/978-1-4939-6481-9_6.
35. Armbruster N, Jasny E, Petsch B. Advances in RNA Vaccines for Preventive Indications: A Case Study of A Vaccine Against Rabies. Vaccines (Basel). (2019); 7(4). Epub 2019 Oct. 2. doi:10.3390/vaccines7040132.
36. Sahin U, Kariko K, Tureci O. mRNA-based therapeutics—developing a new class of drugs. Nat Rev Drug Discov. (2014); 13(10):759-80. Epub 2014 Sep. 23. doi:10.1038/nrd4278.
37. Gebre M S, Brito L A, Tostanoski L H, Edwards D K, Carfi A, Barouch D H. Novel approaches for vaccine development. Cell. (2021); 184(6):1589-1603. Epub 2021 Mar. 20. doi:10.1016/j.cell.2021.02.030.
38. Gary E N, Weiner D B. DNA vaccines: prime time is now. Curr Opin Immunol. (2020); 65:21-27. Epub 2020 Apr. 8. doi:10.1016/j.coi.2020.01.006. Cited in: Pubmed; PMID 32259744.
39. Bergamaschi C, Terpos E, Rosati M, Angel M, Bear J, Stellas D, Karaliota S, Apostolakou F, Bagratuni T, Patseas D, Gumeni S, Trougakos I P, Dimopoulos M A, Felber B K, Pavlakis G N. Systemic IL-15, IFN-gamma, and IP-10/CXCL10 signature associated with effective immune response to SARS-COV-2 in BNT162b2 mRNA vaccine recipients. Cell Rep. (2021); 36:109504. Epub 2021 Aug. 6. doi:10.1016/j.celrep.2021.109504.
40. Hu X, Valentin A, Cai Y, Dayton F, Rosati M, Ramírez-Salazar E G, Kulkarni V, Broderick K E, Sardesai N Y, Wyatt L S, Earl P L, Moss B, Mullins J I, Pavlakis G N, Felber B K. Rapid and High Recall of T Cell Responses upon DNA or rMVA Boost in Macaques Vaccinated with HIV/SIV Conserved Element C E gag DNA Vaccine. Hum Gene Ther. (2018): 29:1029-1043.
41. Kulkarni V, Rosati M, Jalah R, Ganneru B, Alicea C, Yu L, Guan Y, LaBranche C, Montefiori D C, King A D, Valentin A, Pavlakis G N, Felber B K. DNA Vaccination by Intradermal Electroporation Induces Long-lasting Immune Responses in Rhesus Macaques. J Med Primatol (2014); 43 329-40.
42. Kulkarni V, Rosati M, Bear J, Pilkington G R, Jalah R, Bergamaschi C, Singh A K, Alicea C, Chowdhury B, Zhang G-M, Kim E-Y, Wolinsky S M, Huang W, Guan Y, LaBranche C, Montefiori D C, Broderick K E, Sardesai N Y, Valentin A, Felber B K, Pavlakis G N. Comparison of Intradermal and Intramuscular Delivery of SIV Env DNA by in vivo Electroporation in Macaques. Hum Vaccin Immunother. (2013); 9:2081-94.
43. Kulkarni V, Rosati M, Valentin A. Jalah R, Alicea C, Yu L, Guan Y, Shen X, Tomaras G D, LaBranche C, Montefiori D C, Irene C, Prattipati R, Pinter A, Sullivan S M, Pavlakis G N, Felber B K. Vaccination with Vaxfectin® adjuvanted SIV DNA Induces Long-lasting Humoral Immune Responses Able to Reduce SIVmac251 Viremia. Hum Vaccin Immunother. (2013); 9:2069-80.
44. Vasan S, Hurley A, Schlesinger S J, Hannaman D, Gardiner D F, Dugin D P, Boente-Carrera M, Vittorino R, Caskey M, Andersen J, Huang Y, Cox J H, Tarragona-Fiol T, Gill D K, Cheeseman H, Clark L, Dally L, Smith C, Schmidt C, Park H H, Kopycinski J T, Gilmour J, Fast P, Bernard R, Ho D D. In vivo electroporation enhances the immunogenicity of an HIV-1 DNA vaccine candidate in healthy volunteers. PLOS One. (2011); 6(5):e19252. eng. Epub 2011 May 24. doi:10.1371/journal.pone.0019252 PONE-D-11-02192 [pii].
45. Bansal A, Jackson B, West K, Wang S, Lu S, Kennedy J S, Goepfert P A. Multifunctional T-cell characteristics induced by a polyvalent DNA prime/protein boost human immunodeficiency virus type 1 vaccine regimen given to healthy adults are dependent on the route and dose of administration. J Virol. (2008); 82(13):6458-69. Epub 2008 May 2. doi:10.1128/JVI.00068-08.
46. De Rosa S C, Lu F X, Yu J, Perfetto S P, Falloon J, Moser S, Evans T G, Koup R, Miller C J, Roederer M. Vaccination in humans generates broad T cell cytokine responses. J Immunol. (2004); 173(9):5372-80.
47. De Rosa S C, Edupuganti S, Huang Y, Han X, Elizaga M, Swann E, Polakowski L, Kalams S A, Keefer M C, Maenza J, Lu Y, Wise M C, Yan J, Morrow M P, Khan A S, Boyer J D, Humeau L, White S, Pensiero M, Sardesai N Y, Bagarazzi M L, Weiner D B, Ferrari G, Tomaras G D, Montefiori D C, Corey L, McElrath M J, Team HIVVTNS. Robust antibody and cellular responses induced by DNA-only vaccination for HIV. JCI Insight. (2020); 5(13). Epub 2020 May 22. doi:10.1172/jci.insight.137079.
48. Lutz J, Lazzaro S, Habbeddine M, Schmidt K E, Baumhof P, Mui B L, Tam Y K, Madden T D, Hope M J, Heidenreich R, Fotin-Mleczek M. Unmodified mRNA in LNPs constitutes a competitive technology for prophylactic vaccines. NPJ Vaccines. (2017); 2:29. Epub 2017 Dec. 22. doi:10.1038/s41541-017-0032-6.
49. Pardi N, Hogan M J, Pelc R S, Muramatsu H, Andersen H, DeMaso C R, Dowd K A, Sutherland L L, Scearce R M, Parks R, Wagner W, Granados A, Greenhouse J, Walker M, Willis E, Yu J S, McGee C E, Sempowski G D, Mui B L, Tam Y K, Huang Y J, Vanlandingham D, Holmes V M, Balachandran H, Sahu S, Lifton M, Higgs S, Hensley S E, Madden T D, Hope M J. Kariko K, Santra S, Graham B S, Lewis M G, Pierson T C, Haynes B F, Weissman D. Zika virus protection by a single low-dose nucleoside-modified mRNA vaccination. Nature. (2017). doi:10.1038/nature21428.
50. Aldrich C, Leroux-Roels I, Huang K B, Bica M A, Loeliger E, Schoenborn-Kellenberger O, Walz L, Leroux-Roels G, von Sonnenburg F, Oostvogels L. Proof-of-concept of a low-dose unmodified mRNA-based rabies vaccine formulated with lipid nanoparticles in human volunteers: A phase 1 trial. Vaccine. (2021); 39(8):1310-1318. Epub 2021 Jan. 26. doi:10.1016/j.vaccine.2020.12.070.
51. Richner J M, Himansu S, Dowd K A, Butler S L, Salazar V, Fox J M, Julander J G, Tang W W, Shresta S, Pierson T C, Ciaramella G, Diamond M S. Modified mRNA Vaccines Protect against Zika Virus Infection. Cell. (2017); 168(6):1114-1125 e10. Epub 2017 Feb. 23. doi:10.1016/j.cell.2017.02.017.
52. Sahin U, Muik A, Derhovanessian E, Vogler I, Kranz L M, Vormehr M, Baum A, Pascal K, Quandt J, Maurus D, Brachtendorf S. Lorks V, Sikorski J, Hilker R, Becker D, Eller A K, Grutzner J, Boesler C, Rosenbaum C, Kuhnle M C, Luxemburger U, Kemmer-Bruck A, Langer D, Bexon M, Bolte S, Kariko K, Palanche T, Fischer B, Schultz A, Shi P Y, Fontes-Garfias C, Perez J L, Swanson K A, Loschko J, Scully I L, Cutler M, Kalina W, Kyratsous C A, Cooper D, Dormitzer P R, Jansen K U, Tureci O. COVID-19 vaccine BNT162b1 elicits human antibody and TH1 T cell responses. Nature. (2020); 586(7830):594-599. Epub 2020 Oct. 1. doi:10.1038/s41586-020-2814-7.
53. Mulligan M J, Lyke K E, Kitchin N, Absalon J, Gurtman A, Lockhart S, Neuzil K, Raabe V, Bailey R, Swanson K A, Li P, Koury K, Kalina W, Cooper D, Fontes-Garfias C, Shi P Y, Tureci O, Tompkins K R, Walsh E E, Frenck R, Falsey A R, Dormitzer P R, Gruber W C, Sahin U, Jansen K U. Phase I/II study of COVID-19 RNA vaccine BNT162b1 in adults. Nature. (2020); 586(7830):589-593. Epub 2020 Aug. 14. doi:10.1038/s41586-020-2639-4.
54. Widge A T, Rouphael N G, Jackson L A, Anderson E J, Roberts P C, Makhene M, Chappell J D, Denison M R, Stevens L J, Pruijssers A J, McDermott A B, Flach B, Lin B C, Doria-Rose N A, O'Dell S, Schmidt S D, Neuzil K M, Bennett H, Leav B, Makowski M, Albert J, Cross K, Edara V V, Floyd K, Suthar M S, Buchanan W, Luke C J, Ledgerwood J E, Mascola J R, Graham B S, Beigel J H, m RNASG. Durability of Responses after SARS-COV-2 mRNA-1273 Vaccination. N Engl J Med. (2021); 384(1):80-82. Epub 2020 Dec. 4. doi:10.1056/NEJMc2032195.
55. Polack F P, Thomas S J, Kitchin N, Absalon J, Gurtman A, Lockhart S, Perez J L, Perez Marc G, Moreira E D, Zerbini C, Bailey R, Swanson K A, Roychoudhury S, Koury K, Li P, Kalina W V, Cooper D, Frenck R W, Jr., Hammitt L L, Tureci O, Nell H, Schaefer A, Unal S, Tresnan D B, Mather S, Dormitzer P R, Sahin U, Jansen K U, Gruber W C, Group CCT. Safety and Efficacy of the BNT162b2 mRNA Covid-19 Vaccine. N Engl J Med. (2020); 383(27):2603-2615. Epub 2020 Dec. 11. doi:10.1056/NEJMoa2034577.
56. Kremsner P G, Mann P, Kroidl A, Leroux-Roels I, Schindler C, Gabor J J, Schunk M, Leroux-Roels G, Bosch J J, Fendel R, Kreidenweiss A, Velavan T P, Fotin-Mleczek M, Mueller S O, Quintini G, Schonborn-Kellenberger O, Vahrenhorst D, Verstraeten T, Alves de Mesquita M, Walz L, Wolz O O, Oostvogels L, Group C—N—S. Safety and immunogenicity of an mRNA-lipid nanoparticle vaccine candidate against SARS-COV-2: A phase 1 randomized clinical trial. Wien Klin Wochenschr. (2021); 133:931-941. Epub 2021 Aug. 12. doi:10.1007/s00508-021-01922-y.
57. Anderson E J, Rouphael N G, Widge A T, Jackson L A, Roberts P C, Makhene M, Chappell J D, Denison M R, Stevens L J, Pruijssers A J, McDermott A B, Flach B, Lin B C, Doria-Rose N A, O'Dell S, Schmidt S D, Corbett K S, Swanson P A, 2nd, Padilla M, Neuzil K M, Bennett H, Leav B, Makowski M, Albert J, Cross K, Edara V V, Floyd K, Suthar M S, Martinez D R, Baric R, Buchanan W, Luke C J, Phadke V K, Rostad C A, Ledgerwood J E, Graham B S, Beigel J H, m RNASG. Safety and Immunogenicity of SARS-COV-2 mRNA-1273 Vaccine in Older Adults. N Engl J Med. (2020); 383(25):2427-2438. Epub 2020 Sep. 30. doi:10.1056/NEJMoa2028436.
58. Baden L R, El Sahly H M, Essink B, Kotloff K, Frey S, Novak R, Diemert D, Spector S A, Rouphael N, Creech C B, McGettigan J, Khetan S, Segall N, Solis J, Brosz A, Fierro C, Schwartz H, Neuzil K, Corey L, Gilbert P, Janes H, Follmann D, Marovich M, Mascola J, Polakowski L, Ledgerwood J, Graham B S, Bennett H, Pajon R, Knightly C, Leav B, Deng W, Zhou H, Han S, Ivarsson M, Miller J, Zaks T, Group C S. Efficacy and Safety of the mRNA-1273 SARS-COV-2 Vaccine. N Engl J Med. (2021); 384(5):403-416. Epub 2020 Dec. 31. doi:10.1056/NEJMoa2035389.
59. Walsh E E, Frenck R W, Jr., Falsey A R, Kitchin N, Absalon J, Gurtman A, Lockhart S, Neuzil K, Mulligan M J, Bailey R, Swanson K A, Li P, Koury K, Kalina W, Cooper D, Fontes-Garfias C, Shi P Y, Tureci O, Tompkins K R, Lyke K E, Raabe V, Dormitzer P R, Jansen K U, Sahin U, Gruber W C. Safety and Immunogenicity of Two RNA-Based Covid-19 Vaccine Candidates. N Engl J Med. (2020); 383(25):2439-2450. Epub 2020 Oct. 15. doi:10.1056/NEJMoa2027906.
60. van Doremalen N, Fischer R J, Schulz J E, Holbrook M G, Smith B J, Lovaglio J, Petsch B, Munster V J. Immunogenicity of Low-Dose Prime-Boost Vaccination of mRNA Vaccine CV07050101 in Non-Human Primates. Viruses. (2021): 13(8). Epub 2021 Aug. 29. doi:10.3390/v13081645.
61. Rauch S, Roth N, Schwendt K, Fotin-Mleczek M. Mueller S O, Petsch B. mRNA-based SARS-CoV-2 vaccine candidate CVnCOV induces high levels of virus-neutralising antibodies and mediates protection in rodents. NPJ Vaccines. (2021); 6(1):57. Epub 2021 Apr. 18. doi:10.1038/s41541-021-00311-w.
62. Papachristofilou A, Hipp M M, Klinkhardt U, Fruh M, Sebastian M, Weiss C, Pless M, Cathomas R, Hilbe W, Pall G, Wehler T, Alt J, Bischoff H, Geissler M, Griesinger F, Kallen K J, Fotin-Mleczek M, Schroder A, Scheel B, Muth A, Seibel T, Stosnach C, Doener F, Hong H S, Koch S D, Gnad-Vogt U, Zippelius A. Phase Ib evaluation of a self-adjuvanted protamine formulated mRNA-based active cancer immunotherapy, BI1361849 (CV9202), combined with local radiation treatment in patients with stage IV non-small cell lung cancer. J Immunother Cancer. (2019); 7(1):38. Epub 2019 Feb. 10. doi:10.1186/s40425-019-0520-5.
63. Sebastian M, Schroder A, Scheel B, Hong H S, Muth A, von Boehmer L, Zippelius A, Mayer F, Reck M, Atanackovic D. Thomas M, Schneller F, Stohlmacher J, Bernhard H, Groschel A, Lander T, Probst J, Strack T, Wiegand V, Gnad-Vogt U, Kallen K J, Hoerr I, von der Muelbe F, Fotin-Mleczek M, Knuth A, Koch S D. A phase I/IIa study of the mRNA-based cancer immunotherapy CV9201 in patients with stage IIIB/IV non-small cell lung cancer. Cancer Immunol Immunother. (2019); 68(5):799-812. Epub 2019 Feb. 17. doi:10.1007/s00262-019-02315-x.
64. Medina-Magues L G, Gergen J. Jasny E, Petsch B, Lopera-Madrid J, Medina-Magues E S, Salas-Quinchucua C, Osorio J E. mRNA Vaccine Protects against Zika Virus. Vaccines (Basel). (2021); 9(12). Epub 2021 Dec. 29. doi:10.3390/vaccines9121464.
65. Bergamaschi C, Stravokefalou V, Stellas D, Karaliota S, Felber B K, Pavlakis G N. Heterodimeric IL-15 in Cancer Immunotherapy. Cancers (Basel). (2021); 13(4). Epub 2021 Mar. 7. doi:10.3390/cancers 13040837.
66. McGeachy M J, Cua D J, Gaffen S L. The IL-17 Family of Cytokines in Health and Disease. Immunity. (2019); 50(4):892-906. Epub 2019 Apr. 18. doi:10.1016/j.immuni.2019.03.021.
67. Gaffen S L, Jain R, Garg A V, Cua D J. The IL-23-IL-17 immune axis: from mechanisms to therapeutic testing. Nat Rev Immunol. (2014); 14(9):585-600. Epub 2014 Aug. 26. doi:10.1038/nri3707.
68. Langrish C L, Chen Y, Blumenschein W M, Mattson J, Basham B, Sedgwick J D, McClanahan T, Kastelein R A, Cua D J. IL-23 drives a pathogenic T cell population that induces autoimmune inflammation. J Exp Med. (2005); 201(2):233-40. Epub 2005 Jan. 20. doi:10.1084/jem.20041257.
69. Zheng Y, Danilenko D M, Valdez P, Kasman I, Eastham-Anderson J, Wu J, Ouyang W. Interleukin-22, a T (H) 17 cytokine, mediates IL-23-induced dermal inflammation and acanthosis. Nature. (2007); 445(7128):648-51. Epub 2006 Dec. 26. doi:10.1038/nature05505.
70. Jia X B, Zhang Q, Xu L, Yao W J, Wei L. Effect of Malus asiatica Nakai Leaf Flavonoids on the Prevention of Esophageal Cancer in C57BL/6J Mice by Regulating the IL-17 Signaling Pathway. Onco Targets Ther. (2020); 13:6987-6996. Epub 2020 Aug. 9. doi:10.2147/OTT.S261033.
71. Gebre M S, Rauch S, Roth N, Yu J, Chandrashekar A, Mercado N B, He X, Liu J, McMahan K, Martinot A, Martinez D R, Giffin V, Hope D, Patel S, Sellers D, Sanborn O, Barrett J, Liu X, Cole A C, Pessaint L, Valentin D, Flinchbaugh Z, Yalley-Ogunro J, Muench J, Brown R, Cook A, Teow E, Andersen H, Lewis M G, Boon ACM, Baric R S, Mueller S O, Petsch B, Barouch D H. Optimization of non-coding regions for a non-modified mRNA COVID-19 vaccine. Nature. (2022); 601(7893):410-414. Epub 2021 Nov. 19. doi:10.1038/s41586-021-04231-6.
72. Hersperger A R, Martin J N, Shin L Y, Sheth P M, Kovacs C M, Cosma G L, Makedonas G, Pereyra F, Walker B D, Kaul R, Deeks S G, Betts M R. Increased HIV-specific CD8⁺ T-cell cytotoxic potential in HIV elite controllers is associated with T-bet expression. Blood. (2011); 117(14):3799-808. Epub 2011 Feb. 4. doi:10.1182/blood-2010-12-322727.
73. Rosati M, Terpos E, Agarwal M, Karalis V, Bear J, Burns R, Hu X, Papademetriou D, Ntanasis-Stathopoulos I, Trougakos I P, Dimopoulos M A, Pavlakis G N, Felber B K. Distinct Neutralization Profile of Spike Variants by Antibodies Induced upon SARS-COV-2 Infection or Vaccination Am J Hematol. (2022); 97 (1):E3-E7. Epub 2021 Oct. 22. doi:10.1002/ajh.26380.
74. Painter M M, Mathew D, Goel R R, Apostolidis S A, Pattekar A, Kuthuru O, Baxter A E, Herati R S, Oldridge D A, Gouma S, Hicks P, Dysinger S, Lundgreen K A, Kuri-Cervantes L, Adamski S, Hicks A, Korte S, Giles J R, Weirick M E, McAllister C M, Dougherty J, Long S, D'Andrea K, Hamilton J T, Betts M R, Bates P, Hensley S E, Grifoni A, Weiskopf D, Sette A, Greenplate A R, Wherry E J. Rapid induction of antigen-specific CD4(+) T cells is associated with coordinated humoral and cellular immunity to SARS-COV-2 mRNA vaccination. Immunity. (2021); 54(9):2133-2142 e3. Epub 2021 Aug. 29. doi:10.1016/j.immuni.2021.08.001.
75. Arunachalam P S, Wimmers F, Mok C K P, Perera R, Scott M, Hagan T, Sigal N, Feng Y, Bristow L, Tak-Yin Tsang O, Wagh D, Coller J, Pellegrini K L, Kazmin D, Alaaeddine G, Leung W S, Chan J M C, Chik T S H, Choi C Y C, Huerta C, Paine Mccullough M, Lv H, Anderson E, Edupuganti S, Upadhyay A A, Bosinger S E, Maecker H T, Khatri P, Rouphael N, Peiris M, Pulendran B. Systems biological assessment of immunity to mild versus severe COVID-19 infection in humans. Science. (2020); 369(6508):1210-1220. Epub 2020 Aug. 14. doi:10.1126/science.abc6261.
76. Fourati S, Ribeiro S P, Blasco Tavares Pereira Lopes F, Talla A, Lefebvre F, Cameron M, Kaewkungwal J, Pitisuttithum P, Nitayaphan S, Rerks-Ngarm S, Kim J H, Thomas R, Gilbert P B, Tomaras G D, Koup R A, Michael N L, McElrath M J, Gottardo R, Sekaly R P. Integrated systems approach defines the antiviral pathways conferring protection by the RV144 HIV vaccine. Nat Commun. (2019); 10(1):863. Epub 2019 Feb. 23. doi:10.1038/s41467-019-08854-2.
77. Hagan T, Pulendran B. Will Systems Biology Deliver Its Promise and Contribute to the Development of New or Improved Vaccines? From Data to Understanding through Systems Biology. Cold Spring Harb Perspect Biol. (2018); 10(8):a028894. Epub 2017 Oct. 19. doi:10.1101/cshperspect.a028894.
78. Kuri-Cervantes L, Fourati S, Canderan G, Sekaly R P. Systems biology and the quest for correlates of protection to guide the development of an HIV vaccine. Curr Opin Immunol. (2016); 41:91-97. Epub 2016 Jul. 9. doi:10.1016/j.coi.2016.06.010.
79. Querec T D, Akondy R S, Lee E K, Cao W, Nakaya H I, Teuwen D, Pirani A, Gernert K, Deng J, Marzolf B, Kennedy K, Wu H, Bennouna S, Oluoch H, Miller J, Vencio R Z, Mulligan M, Aderem A, Ahmed R, Pulendran B. Systems biology approach predicts immunogenicity of the yellow fever vaccine in humans. Nat Immunol. (2009); 10(1):116-125. Epub 2008 Nov. 26. doi:10.1038/ni.1688.
80. Zak D E, Andersen-Nissen E, Peterson E R, Sato A, Hamilton M K, Borgerding J, Krishnamurty A T, Chang J T, Adams D J, Hensley T R, Salter A1, Morgan C A, Duerr A C, De Rosa S C, Aderem A, McElrath M J. Merck Ad5/HIV induces broad innate immune activation that predicts CD8(+) T-cell responses but is attenuated by preexisting Ad5 immunity. Proc Natl Acad Sci USA. (2012); 109(50):E3503-12. Epub 2012 Nov. 16. doi:10.1073/pnas. 1208972109.
81. Andersen-Nissen E, Fiore-Gartland A, Ballweber Fleming L, Carpp L N, Naidoo A F, Harper M S, Voillet V, Grunenberg N, Laher F, Innes C, Bekker L G, Kublin J G, Huang Y, Ferrari G, Tomaras G D, Gray G, Gilbert P B, McElrath M J. Innate immune signatures to a partially-efficacious HIV vaccine predict correlates of HIV-1 infection risk. PLOS Pathog. (2021); 17(3):e1009363. Epub 2021 Mar. 16. doi:10.1371/journal.ppat.1009363.
82. Gaucher D, Therrien R, Kettaf N, Angermann B R, Boucher G, Filali-Mouhim A, Moser J M, Mehta R S, Drake D R, 3rd, Castro E, Akondy R, Rinfret A, Yassine-Diab B, Said E A, Chouikh Y, Cameron M J, Clum R, Kelvin D, Somogyi R, Greller L D, Balderas R S, Wilkinson P, Pantaleo G, Tartaglia J, Haddad E K, Sekaly R P. Yellow fever vaccine induces integrated multilineage and polyfunctional immune responses. J Exp Med. (2008); 205(13):3119-31. Epub 2008 Dec. 3. doi:10.1084/jem.20082292.
83. Korn T, Bettelli E, Oukka M, Kuchroo V K. IL-17 and Th17 Cells. Annu Rev Immunol. (2009); 27:485-517. Epub 2009 Jan. 10. doi:10.1146/annurev.immunol.021908.132710.
84. Perera P Y, Lichy J H, Waldmann T A, Perera L P. The role of interleukin-15 in inflammation and immune responses to infection: implications for its therapeutic use. Microbes Infect. (2012); 14(3):247-61. Epub 2011 Nov. 9. doi:10.1016/j.micinf.2011.10.006.
85. Stone S L, Peel J N, Scharer C D, Risley C A, Chisolm D A, Schultz M D, Yu B, Ballesteros-Tato A, Wojciechowski W, Mousseau B, Misra R S, Hanidu A, Jiang H, Qi Z, Boss J M, Randall T D, Brodeur S R, Goldrath A W, Weinmann A S, Rosenberg A F, Lund F E. T-bet Transcription Factor Promotes Antibody-Secreting Cell Differentiation by Limiting the Inflammatory Effects of IFN-gamma on B Cells. Immunity. (2019); 50(5):1172-1187 e7. Epub 2019 May 12. doi:10.1016/j.immuni.2019.04.004.
86. Baumgarth N. The Shaping of a B Cell Pool Maximally Responsive to Infections. Annu Rev Immunol. (2021); 39:103-129. Epub 2021 Jan. 21. doi:10.1146/annurev-immunol-042718-041238.
87. Bergamaschi C, Pandit H, Nagy B A, Stellas D, Jensen S M, Bear J, Cam M, Valentin A, Fox B A, Felber B K, Pavlakis G N. Heterodimeric IL-15 delays tumor growth and promotes intratumoral CTL and dendritic cell accumulation by a cytokine network involving XCL1, IFN-gamma, CXCL9 and CXCL 10. J Immunother Cancer. (2020); 8(1):e000599. Epub 2020 May 29. doi:10.1136/jitc-2020-000599.

1. A method of inducing an immune response to a protein of interest in a subject, the method comprising:
(a) administering one or more priming doses comprising a DNA construct encoding at least a portion of the protein of interest;
(b) administering one or more boosting doses comprising a lipid nanoparticle (LNP) comprising an RNA construct encoding at least a portion of the protein of interest;
wherein the one or more boosting doses is administered about 2 weeks to about 4 years after the last of the one or more priming doses.

2. A method of inducing an immune response to a protein of interest in a subject, the method comprising:
(a) administering one or more priming doses comprising a lipid nanoparticle (LNP) comprising an RNA construct encoding at least a portion of the protein of interest;
(b) administering one or more boosting doses comprising a DNA construct encoding at least a portion of the protein of interest;
wherein the one or more boosting doses is administered about 2 weeks to about 4 years after the last of the one or more priming doses.

3. The method of claim 1, wherein the one or more priming doses, the one or more boosting doses, or both the one or more priming doses and the one or more boosting doses comprises one or more adjuvants.

4. The method of claim 1, wherein the one or more adjuvants are selected from the group consisting of Adju-Phos™, Adjumer™, albumin-heparin microparticles, Algammulin, AS-2 adjuvant, Avridine™, B7-2, BAK, BAY R1005, Bupivacaine, Bupivacaine-HCl, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CRL1005, D-Murapalmitine, Diphtheria toxoid, DMPC, DMPG, Freund's Complete Adjuvant, GM-CSF, GMDP, hGM-CSF, hIL-12 (N222L), hTNF-alpha, IFA, Imiquimod™, ImmTher™, Interferon-gamma, Interleukin-1 beta, Interleukin-12, Interleukin-2, Interleukin-4, Interleukin-7, ISCOM(s)™, Iscoprep 7.0.3™, Loxoribine, LT (R192G), LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, S-28463, SAF-1, Sclavo peptide, Span 85, Specol, Tetanus toxoid (TT), Theramide™, Threonyl muramyl dipeptide (TMDP), and Ty Particles.

5. The method of claim 1, wherein each dose of the one or more priming doses comprising the DNA construct comprises about 1 mg to about 20 mg of the DNA construct.

6. The method of claim 2, wherein each dose of the one or more boosting doses comprising the DNA construct comprises about 1 mg to about 20 mg of the DNA construct.

7. The method of claim 1, wherein each dose of the one or more boosting doses comprising the RNA construct comprises about 1 μg to about 100 μg of the RNA construct.

8. The method of claim 2, wherein each dose of the one or more priming doses comprising the RNA construct comprises about 1 μg to about 100 μg of the RNA construct.

9. The method of claim 1, wherein the one or more priming doses, the one or more boosting doses, or both the one or more priming doses and the one or more boosting doses is administered by intramuscular injection, intramuscular injection followed by in vivo electroporation, subcutaneous injection, intravenous injection, or by inhalation.

10. The method of claim 1, wherein the protein of interest is HIV-1 Gag or one or more conserved elements from HIV-1 p24^{gag}.

11. The method of claim 1, wherein the protein of interest encoded by the DNA construct or the RNA construct is the same protein.

12. The method of claim 1, wherein the protein of interest encoded by the DNA construct or the RNA construct are different proteins, for example, comprising one or more conserved elements, fragments, or variants of the protein of interest.

13. The method of claim 1, wherein the one or more priming doses comprises two, three, four, or five doses or more, each separated by at least about 2 weeks, 3 weeks, 4 weeks, 5 weeks, 6 weeks, 7 weeks, 8 weeks, or more.

14. The method of claim 1, wherein the one or more boosting doses comprises two, three, four, or five doses or more, each separated by at least about 2 weeks, 3 weeks, 4 weeks, 5 weeks, 6 weeks, 7 weeks, 8 weeks, or more.

15. The method of claim 1, wherein the one or more boosting doses is administered at least about 2 weeks, 3 weeks, 4 weeks, 5 weeks, 6 weeks, 7 weeks, 8 weeks, or more after the last of the one or more priming doses, or wherein the one or more boosting doses is administered at least about 3 months, 4 months, 5 months, 6 months, 7 months, 8 months, 9 months, 10 months, 11 months, 12 months or more after the last of the one or more priming doses.

16. A lipid nanoparticle (LNP), comprising an RNA molecule encoding HIV-1 Gag or one or more conserved elements from HIV-1 p24^{gag}.

17. The lipid nanoparticle of claim 16, wherein the LNP comprises about 1 μg to about 100 μg of the RNA molecule.

18. The lipid nanoparticle of claim 16, wherein the LNP comprises a second RNA molecule encoding one or more cytokines selected from IL-12, IL-7, IL-15, and IL-21.


SEQUENCE LISTING (5 sequences)

SEQ ID NO: 1 - DNA, 5525 bp, synthetic construct
  source 1..5525: mol_type = other DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgaagaaatgggggcgcgggcctcggtccttagcgggggcgagttggatcggtgggaaaagatccgcttgaggccaggagggaagaagaagtacaagctaaagcacatcgtctgggcgagcagagagttggagcggttcgcggtcaacccgggcctgcttgagacatcggagggctgtcggcaaatcctggggcagcttcaaccgtccttgcaaacgggcagcgaggagcttcgatcactatacaacactgtagcaacgctctactgcgtgcaccagcggatcgagatcaaggacacgaaggaggctcttgacaagattgaggaagagcagaacaagtccaagaagaaggcccagcaggcggcggccgacaccggccactccaaccaagtatcacagaactacccgatcgtgcagaacatccagggacagatggtccaccaggccatctccccacggacgcttaacgcgtgggtcaaagtagtggaggagaaggccttcagcccggaagtgatccccatgttctcggcactttccgagggagccaccccgcaggacctgaacacgatgttgaacaccgtcggcgggcaccaggcggccatgcagatgcttaaggagaccatcaacgaggaggctgcggagtgggaccgggtccacccggtgcacgcggggcccatcgcgccgggccagatgagagagccgcggggatcggacatcgcgggaaccaccagcaccttgcaggagcaaatcggttggatgactaacaacccgccaatcccggtcggggagatctacaagagatggatcatcctcgggttgaacaagatcgtgaggatgtacagcccgaccagcatcctggacatccgacagggaccgaaggagccgttcagagactacgtagaccggttctacaagactctccgggcggagcaggcgtcgcaggaggtcaagaactggatgacggagaccttgttggtccagaacgcgaacccggactgcaagaccatcctgaaggctctcggcccggcggcgacgttggaagagatgatgacggcgtgccagggagtcgggggacccggccacaaggcgcgggtcttggccgaggcgatgagccaagtgacgaactcggcgacgatcatgatgcagcggggcaacttccggaaccagcggaagatcgtcaagtgcttcaactgtggcaaggagggacacaccgccaggaactgccgggccccccggaagaagggctgctggaagtgcggaaaggaggggcaccaaatgaaggactgcacggagcggcaggcgaatttcctcgggaagatctggccgtcctacaaggggcggccagggaactttctgcaaagccggccggagccgaccgccccgccggaggagtcctttcggtccggggtcgagacgaccacgccccctcagaagcaagagcccatcgacaaggagttgtaccctcttacctccctccggtcgctcttcggcaacgacccgtcctcgcaatgataagaattcgagctcggtacgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagagacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 2 - protein, 500 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 3 - DNA, 5577 bp, synthetic construct
  source 1..5577: mol_type = other DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacaagaaatgtggctccagagcctgctactcctggggacggtggcctgcagcatctcggtcatcccgatgttctcggcgctcagcgagggagcgacgccgcaggacctgaacgcggccgtcggaggtcaccaggcagcgatgcagatgctgaaggacacgatcaacgaggaggcggccgagtgggaccgggcggcagccgagccacgcggttccgacatcgcgggcaccacctcgacgctccaggagcagatcgggtgggccgcagctaagcgctggatcatcctcgggctgaacaagatcgtccggatgtacagcccgacgtcgatcgctgctaagtacgttgaccggttctacaagaccctgagggccgagcaggcggccggactggaggagatgatgaccgcgtgccagggggtcggtggaccagggcacaaggccgcgatctcgccgcgcacgctgaacgcgtgggtgaaggtctgataagaattcgctagcggcgcgccagatctgatatcggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggatcatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgtcgaggatccggcttatcagaccttcacccaggcgttgagggtgcgaggcgagagggccgccttgtgcgacggtcctccgactccctggcaggctgtcatcatctcctcgagacccgcggcctgctctgccctcagcgtcttgaagaagcggtctacgtatttggccgcgatgctgactgggctgtacatcctgacgatcttgttgaggcccaggatgatccagcgcttggctgcagcccaggcgatctgctcctggagggtgctggtcgtgcctgcgatgtcgctaccccttggctcagctgctgccctgtcccactcggctgcctcctcgttgatggtctccttgagcatctgcattgccgcctggtgtccaccgaccgcggcgttgaggtcctgcggtgtcgcaccctcactgagtgcggtgaacatggggatgaccgagatcgagcacgccacggtcccgagtagcaggagcgactgcagccacatttcttgccgtttaaacgtcgacagatccaaacgctcctccgacgtccccaggcagaatggcggttccctaaacgagcattgcttatatagacctcccattaggcacgcctaccgcccatttacgtcaatggaacgcccatttgcgtcattgcccctccccattgacgtcaatggggatgtacttggcagccatcgcgggccatttaccgccattgacgtcaatgggagtactgccaatgtaccctggcgtacttccaatagtaatgtacttgccaagttactattaatagatattgatgtactgccaagtgggccatttaccgtcattgacgtcaatagggggcgtgagaacggatatgaatgggcaatgagccatcccattgacgtcaatggtgggtggtcctattgacgtcaatgggcattgagccaggcgggccatttaccgtaattgacgtcaatgggggaggcgccatatacgtcaataggaccgcccatatgacgtcaataggtaagaccatgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 4 - protein, 157 aa, synthetic construct
MWLQSLLLLGTVACSISVIPMFSALSEGATPQDLNAAVGGHQAAMQMLKDTINEEAAEWDRAAAEPRGSDIAGTTSTLQEQIGWAAAKRWIILGLNKIVRMYSPTSIAAKYVDRFYKTLRAEQAAGLEEMMTACQGVGGPGHKAAISPRTLNAWVKV

SEQ ID NO: 5 - protein, 157 aa, synthetic construct
MWLQSLLLLGTVACSISVIPMFTALSEGATPQDLNAAVGGHQAAMQMLKETINEEAAEWDRAAAEPRGSDIAGTTSTLQEQIAWAAAKRWIILGLNKIVRMYSPVSIAAKYVDRFFKTLRAEQAAGLEEMMTACQGVGGPSHKAALSPRTLNAWVKV